(19) United States
(12) Patent Application Publication
(10) Pub. No.: US 2023/0202980 A1
(43) Pub. Date: Jun. 29, 2023

(54) N-(2-Aminophenyl)-Prop-2-Enamide Derivatives, and Uses Thereof in the Treatment of Cancer

(71) Applicants: National University of Singapore, Singapore (SG); The Brigham and Women’s Hospital, Inc., Boston, MA (US); Indian Institute of Chemical Technology, Hyderabad (IN)

(72) Inventors: Sridhar Radhakrishnan; Daniel G. Tenen; Bee Hui Liu; Kim Anh Vu Le; Mei Lin Go; Li Chai, Boston, MA (US); Chong Gao, Boston, MA (US); Ahmed Kamal, Hyderabad (IN); Satish Sunkari, Hyderabad (IN); Venkata Subbarao Ayinampudi, Hyderabad (IN); Riyaz Syed, Hyderabad (IN); Miao Liu, Boston, MA (US)

(21) Appl. No.: 17/638,521

(22) PCT Filed: Aug. 28, 2020
(86) PCT No.: PCT/US2020/048477

§ 371 Date: Feb. 25, 2022

Related U.S. Application Data
(60) Provisional application No. 62/894,189, filed on Aug. 30, 2019.

(51) Int. Cl. C07D 213/72; C07D 401/12; C07D 401/14; C07D 417/14; A61P 35/00

(52) U.S. Cl. CPC C07D 213/72; A61P 35/00; C07D 401/12; C07D 401/14; C07D 417/14

(57) ABSTRACT

Provided herein are N-(2-aminophenyl)-prop-2-enamide derivatives, such as those of Formula (I), methods for the synthesis thereof, and uses thereof in the treatment of cancer, such as SALL4-expressing cancer, in a cell or subject in need thereof.

### FIELD OF TECHNOLOGY

The following relates generally to anticancer agents. More specifically, the following relates to N-(2-aminophenyl)-prop-2-enamide derivatives, and uses thereof in the treatment of cancer.

### BACKGROUND

Embryonic factor SALL4 is mainly expressed in fetal and stem cells, and has been found to be aberrantly expressed in many cancers. Inhibition of the SALL4-NuRD complex may provide a druggable target for the treatment of cancers, especially lung and liver cancers.

Approximately 10 SALL4- or FOG1-derived peptides have been shown to inhibit the SALL4-NuRD interaction at sub-micromolar concentrations. Although in vivo efficacy has been shown for these peptides, they suffer from poor drug-like properties, especially permeability and metabolic stability (i.e. cell penetration and bioavailability).

To identify improved small molecule inhibitors of SALL4-RBBP4, 14000 small molecules were screened on AlphaScreen, cell viability WSTs (Water-soluble Tetrazolium salts), thermal shift and fluorescent polarisation assays. With oncofetal protein SALL4 screened as a cancer target, Chai et al have reported application of the HDAC inhibitor entinostat as a potential drug [1-5] in treating SALL4-expressing cancers, particularly NSCLC, and this was confirmed in 17 lung cancer cell lines. Entinostat is under active study and may show promise against certain cancers; however, the problem of cancer remains a major health concern, and additional anticancer drugs are highly desirable in the field.

Alternative, additional, and/or improved anticancer agents, methods for the production thereof, and/or uses thereof, are desirable.

### SUMMARY

Provided herein are N-(2-aminophenyl)-prop-2-enamide derivatives having anticancer activity. Extensive structure-activity studies were performed as described herein, and potent compounds and pharmacophores identified. In certain embodiments, examples of N-(2-aminophenyl)-prop-2-enamide derivatives as described herein may provide potent anticancer and/or growth inhibitory activity, which may be selective for cells (such as lung cancer cells or liver cancer cells) having an elevated or high level of SALL4 expression as compared with cells having low SALL4 levels.

In an embodiment, there is provided herein a compound of Formula I:

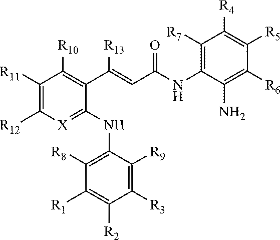

wherein
R₁ is H or —OCH₃;
R₂ is H, —OCH₃, —CF₃, or F;
R₃ is H or —OCH₃;
R₄ is H, Cl, —OCH₃, —CH₃, F, or —NH₂;
R₅ is H, Cl, —OCH₃, —CH₃, F, or —NH₂;
R₆ is H, Cl, —OCH₃, or —CH₃;
R₇ is H, Cl, —OCH₃, or —CH₃;
R₈ is H, —OCH₃, or —CH₃;
R₉ is H, —OCH₃, or —CH₃;
each of R₁₀, R₁₁, and R₁₂ is H, or R₁₀ and R₁₁ together with the carbon atoms to which they are attached form a 5 or 6-membered aryl or heteroaryl ring and R₁₂ is H, or R₁₁ and R₁₂ together with the carbon atoms to which they are attached form a 5 or 6-membered aryl or heteroaryl ring and R₁₀ is H;
R₁₃ is H, —C≡C—CH₃, —C≡C—C₃H₅, or —C≡N; and
X is N or CH.

In another embodiment of the above compound, R₁ may be -OCH₃.

In yet another embodiment of any of the above compound or compounds, R₂ may be -OCH₃ or -CF₃.

In yet another embodiment of any of the above compound or compounds, R₃ may be -OCH₃.

In still another embodiment of any of the above compound or compounds, R₄ may be H, Cl, -OCH₃, or -CH₃.

In yet another embodiment of any of the above compound or compounds, R₅ may be H or Cl.

In another embodiment of any of the above compound or compounds, R₆ may be H.

In yet another embodiment of any of the above compound or compounds, R₇ may be H.

In still another embodiment of any of the above compound or compounds, R₈ may be H.

In yet another embodiment of any of the above compound or compounds, R₉ may be H.

In yet another embodiment of any of the above compound or compounds, R₁₀, R₁₁, and R₁₂ are each H.

In another embodiment of any of the above compound or compounds, R₁₃ may be H.

In yet another embodiment of any of the above compound or compounds, X may be N.

In yet another embodiment of any of the above compound or compounds, the compound may be:

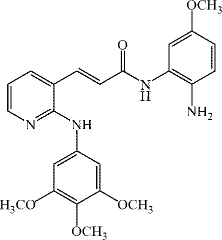 [SMILES: COc1ccc(N)c(NC(=O)/C=C/c2cccnc2Nc2cc(OC)c(OC)c(OC)c2)c1]

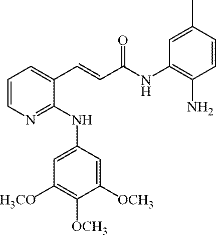 [SMILES: COc1cc(Nc2ncccc2/C=C/C(=O)Nc2cc(C)ccc2N)cc(OC)c1OC]

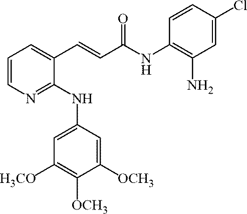 [SMILES: COc1cc(Nc2ncccc2/C=C/C(=O)Nc2ccc(Cl)cc2N)cc(OC)c1OC]

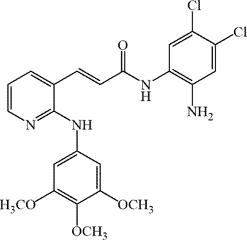 [SMILES: COc1cc(Nc2ncccc2/C=C/C(=O)Nc2cc(Cl)c(Cl)cc2N)cc(OC)c1OC]

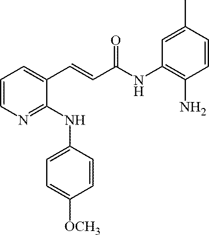 [SMILES: COc1ccc(Nc2ncccc2/C=C/C(=O)Nc2cc(C)ccc2N)cc1]

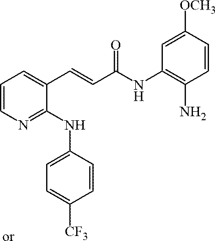

In yet another embodiment of any of the above compound or compounds, the compound may be:

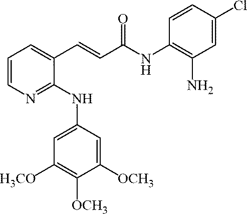 [SMILES: COc1cc(Nc2ncccc2/C=C/C(=O)Nc2ccc(Cl)cc2N)cc(OC)c1OC]

In another embodiment, there is provided herein a composition comprising any one or more of the compounds described herein, and a pharmaceutically acceptable carrier, excipient, or diluent.

In another embodiment, any of the compounds or compositions as described herein may be for use in the treatment of cancer in a cell or a subject in need thereof.

In another embodiment, there is provided herein a use of any of the compounds or compositions as described herein for the treatment of cancer in a cell or a subject in need thereof

In still another embodiment, there is provided herein a use of any of the compounds or compositions as described herein in the manufacture of a medicament for use in the treatment of cancer in a cell or a subject in need thereof.

In another embodiment of any of the above compounds/compositions for use, or uses, the cancer may be a SALL4-expressing cancer. In another embodiment, the cancer may be a SALL4-expressing cancer having a high level of SALL4 expression. In still another embodiment, the cancer may be lung cancer, liver cancer, or breast cancer. In yet another embodiment, the cancer may be NSCLC cancer, cervical cancer, or germ cell cancer.

In still another embodiment, there is provided herein a method for treating cancer in a cell or subject in need thereof, said method comprising:

administering any of the compound or compounds as described herein, or a composition as described herein, to the cell or subject.

In another embodiment of the above method, the cancer may be a SALL4-expressing cancer. In yet another embodiment, the cancer may be a SALL4-expressing cancer having a high level of SALL4 expression. In still another embodiment, the cancer may be lung cancer, liver cancer, or breast cancer. In yet another embodiment, the cancer may be NSCLC cancer, cervical cancer, or germ cell cancer.

In another embodiment, there is provided herein a method for preparing an N-(2-aminophenyl)-prop-2-enamide derivative, said method comprising:
reacting a compound of formula 1 with a compound of formula 2 to form a compound of formula 3:

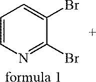

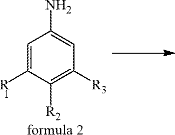

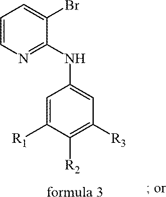

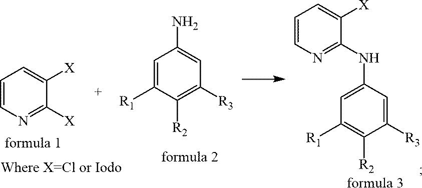

reacting the compound of formula 3 with a compound of formula 4, and deprotecting, to form a compound of formula 5:

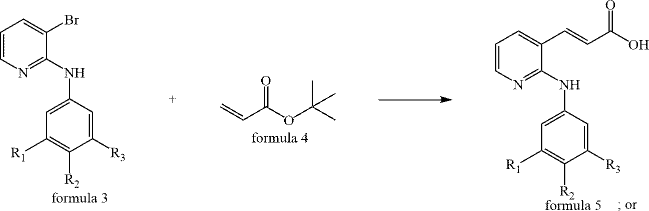

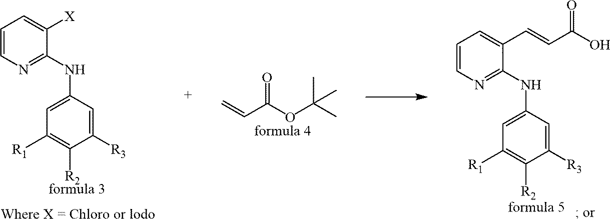

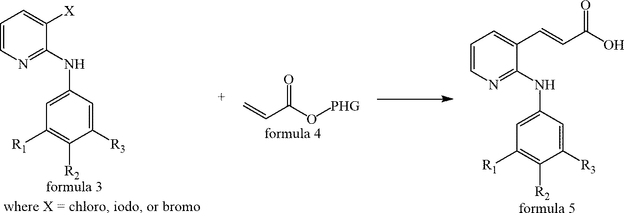

where PG represents any suitable protecting group known to the person of skill in the art having regard to the teachings herein (such as, but not limited to, an alkyl ester such methyl ester, ethyl ester, or t-butyl ester), and deprotecting includes removal of the PG protecting group to form a compound of formula 5 (deprotecting conditions may be selected based on the protecting group used - examples may include, for example, an acidic or basic hydrolysis to yield formula 5); and
reacting a compound of formula 5 with a compound of formula 6 to form an N-(2-aminophenyl)-prop-2-enamide derivative of formula 7:

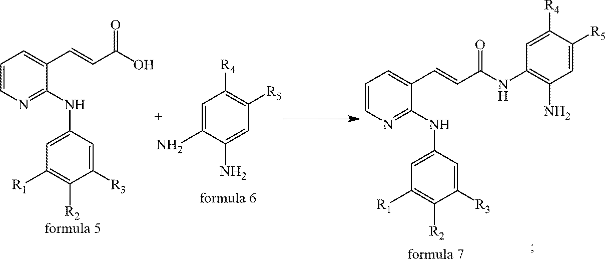

wherein
R₁, R₂, and R₃ are each independently selected from H, —OCH₃, —CF₃, Cl, or F;
R₄ is H, Cl, —OCH₃, —CH₃, or F; and
R₅ is H, Cl, —OCH₃, —CH₃, or F.

In an alternative embodiment of the above method, the compound of formula 5 may instead be prepared by reacting a compound of formula 3b with a compound of formula 2:

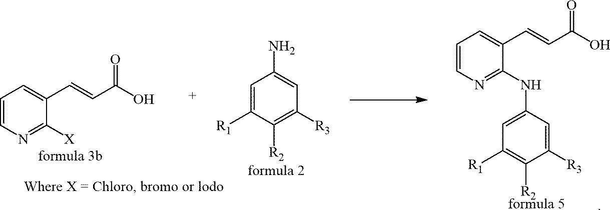

In another embodiment of the above method, the compound of formula 1 may be reacted with the compound of formula 2 in NaOBu-t, Pd(OAc)₂, PPh₃, and xylene.

In still another embodiment of any of the above method or methods, the compound of formula 3 may be reacted with the compound of formula 4 in Pd₂(dba)₃, DMF, and DIPEA.

In yet another embodiment of any of the above method or methods, deprotection to form a compound of formula 5 may be performed with TFA in DCM.

In still another embodiment of any of the above method or methods, the compound of formula 5 may be reacted with the compound of formula 6 in BOP, Et₃N, and MeCN.

In another embodiment, there is provided herein a compound of formula 7 produced by any of the method or methods described herein. In another embodiment, the compound of formula 7 may be

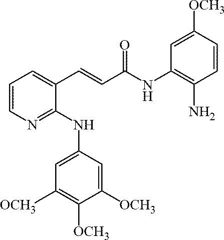 [SMILES: COc1ccc(N)c(NC(=O)/C=C/c2cccnc2Nc2cc(OC)c(OC)c(OC)c2)c1]

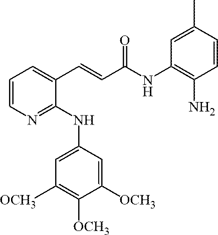 [SMILES: COc1cc(Nc2ncccc2/C=C/C(=O)Nc2cc(C)ccc2N)cc(OC)c1OC]

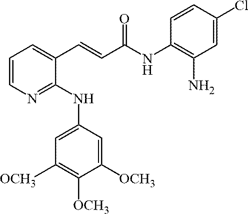 [SMILES: COc1cc(Nc2ncccc2/C=C/C(=O)Nc2ccc(Cl)cc2N)cc(OC)c1OC]

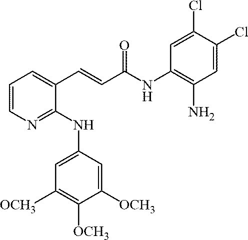 [SMILES: COc1cc(Nc2ncccc2/C=C/C(=O)Nc2cc(Cl)c(Cl)cc2N)cc(OC)c1OC]

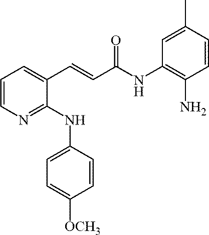 [SMILES: COc1ccc(Nc2ncccc2/C=C/C(=O)Nc2cc(C)ccc2N)cc1]

or

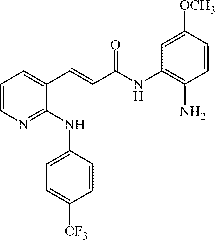 [SMILES: COc1ccc(N)c(NC(=O)/C=C/c2cccnc2Nc2ccc(C(F)(F)F)cc2)c1]

In still another embodiment, there is provided herein a compound which may be any one of the following:

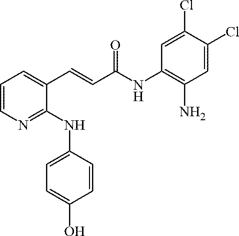 [SMILES: Nc1cc(Cl)c(Cl)cc1NC(=O)/C=C/c1cccnc1Nc1ccc(O)cc1]

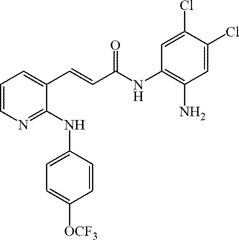 [SMILES: Nc1cc(Cl)c(Cl)cc1NC(=O)/C=C/c1cccnc1Nc1ccc(OC(F)(F)F)cc1]

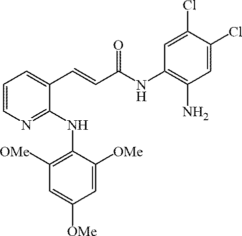 [SMILES: COc1cc(OC)c(Nc2ncccc2/C=C/C(=O)Nc2cc(Cl)c(Cl)cc2N)c(OC)c1]

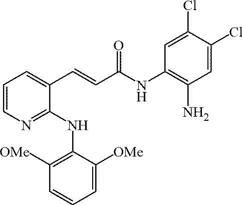 [SMILES: COc1cccc(OC)c1Nc1ncccc1/C=C/C(=O)Nc1cc(Cl)c(Cl)cc1N]

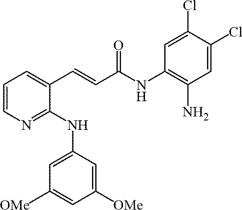 [SMILES: COc1cc(Nc2ncccc2/C=C/C(=O)Nc2cc(Cl)c(Cl)cc2N)cc(OC)c1]

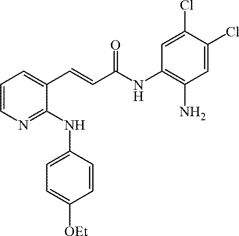 [SMILES: CCOc1ccc(Nc2ncccc2/C=C/C(=O)Nc2cc(Cl)c(Cl)cc2N)cc1]

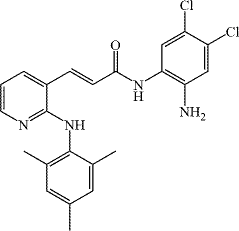 [SMILES: Cc1cc(C)c(Nc2ncccc2/C=C/C(=O)Nc2cc(Cl)c(Cl)cc2N)c(C)c1]

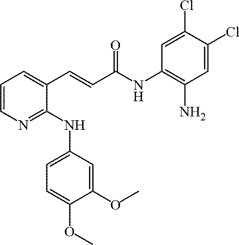 [SMILES: COc1ccc(Nc2ncccc2/C=C/C(=O)Nc2cc(Cl)c(Cl)cc2N)cc1OC]

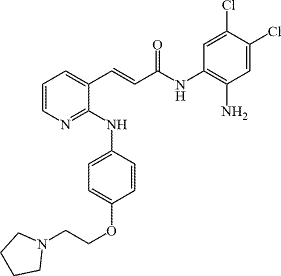 [SMILES: Nc1cc(Cl)c(Cl)cc1NC(=O)/C=C/c1cccnc1Nc1ccc(OCCN2CCCC2)cc1]

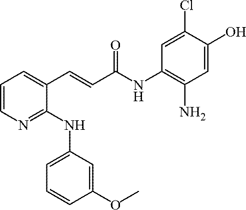 [SMILES: COc1cccc(Nc2ncccc2/C=C/C(=O)Nc2cc(Cl)c(O)cc2N)c1]

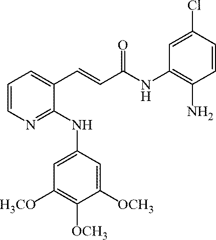 [SMILES: COc1cc(Nc2ncccc2/C=C/C(=O)Nc2cc(Cl)ccc2N)cc(OC)c1OC]

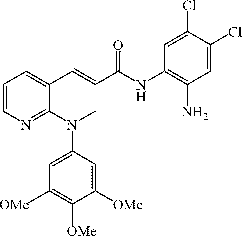 [SMILES: COc1cc(N(C)c2ncccc2/C=C/C(=O)Nc2cc(Cl)c(Cl)cc2N)cc(OC)c1OC]

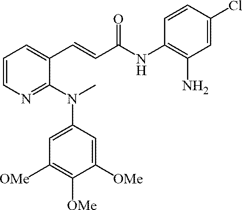 [SMILES: COc1cc(N(C)c2ncccc2/C=C/C(=O)Nc2ccc(Cl)cc2N)cc(OC)c1OC]

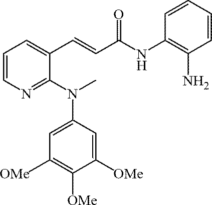 [SMILES: COc1cc(N(C)c2ncccc2/C=C/C(=O)Nc2ccccc2N)cc(OC)c1OC]

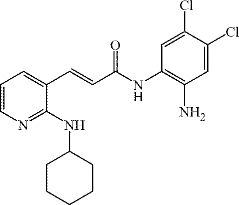 [SMILES: Nc1cc(Cl)c(Cl)cc1NC(=O)/C=C/c1cccnc1NC1CCCCC1]

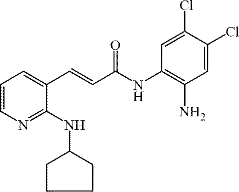 [SMILES: Nc1cc(Cl)c(Cl)cc1NC(=O)/C=C/c1cccnc1NC1CCCC1]

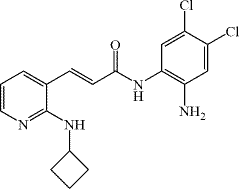 [SMILES: Nc1cc(Cl)c(Cl)cc1NC(=O)/C=C/c1cccnc1NC1CCC1]

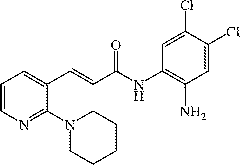 [SMILES: Nc1cc(Cl)c(Cl)cc1NC(=O)/C=C/c1cccnc1N1CCCCC1]

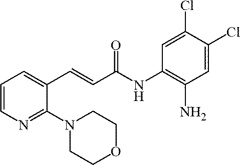 [SMILES: Nc1cc(Cl)c(Cl)cc1NC(=O)/C=C/c1cccnc1N1CCOCC1]

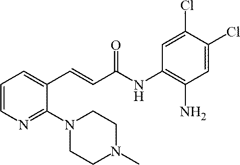 [SMILES: CN1CCN(c2ncccc2/C=C/C(=O)Nc2cc(Cl)c(Cl)cc2N)CC1]

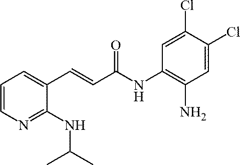 [SMILES: CC(C)Nc1ncccc1/C=C/C(=O)Nc1cc(Cl)c(Cl)cc1N]

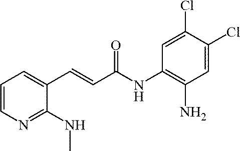 [SMILES: CNc1ncccc1/C=C/C(=O)Nc1cc(Cl)c(Cl)cc1N]

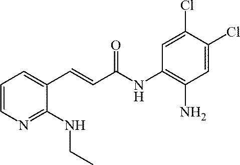 [SMILES: CCNc1ncccc1/C=C/C(=O)Nc1cc(Cl)c(Cl)cc1N]

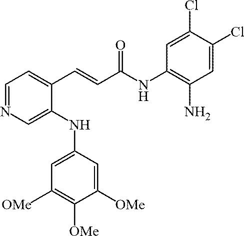 [SMILES: COc1cc(Nc2cnccc2/C=C/C(=O)Nc2cc(Cl)c(Cl)cc2N)cc(OC)c1OC]

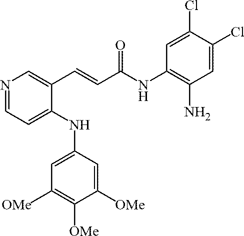 [SMILES: COc1cc(Nc2ccncc2/C=C/C(=O)Nc2cc(Cl)c(Cl)cc2N)cc(OC)c1OC]

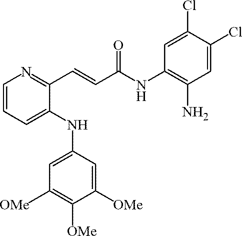 [SMILES: COc1cc(Nc2cccnc2/C=C/C(=O)Nc2cc(Cl)c(Cl)cc2N)cc(OC)c1OC]

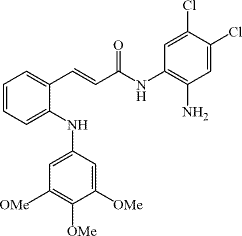 [SMILES: COc1cc(Nc2ccccc2/C=C/C(=O)Nc2cc(Cl)c(Cl)cc2N)cc(OC)c1OC]

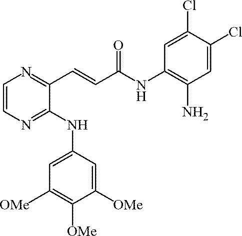 [SMILES: COc1cc(Nc2nccnc2/C=C/C(=O)Nc2cc(Cl)c(Cl)cc2N)cc(OC)c1OC]

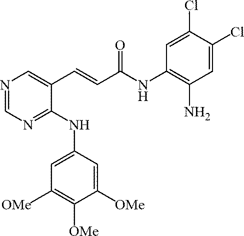 [SMILES: COc1cc(Nc2ncncc2/C=C/C(=O)Nc2cc(Cl)c(Cl)cc2N)cc(OC)c1OC]

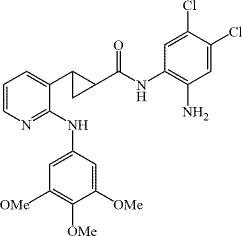 [SMILES: COc1cc(Nc2ncccc2C2CC2C(=O)Nc2cc(Cl)c(Cl)cc2N)cc(OC)c1OC]

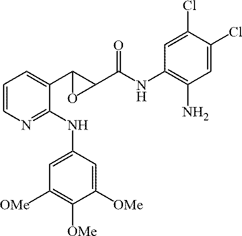 [SMILES: COc1cc(Nc2ncccc2C2OC2C(=O)Nc2cc(Cl)c(Cl)cc2N)cc(OC)c1OC]

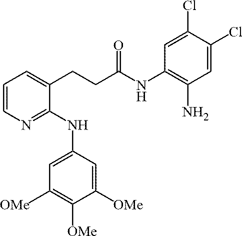 [SMILES: COc1cc(Nc2ncccc2CCC(=O)Nc2cc(Cl)c(Cl)cc2N)cc(OC)c1OC]

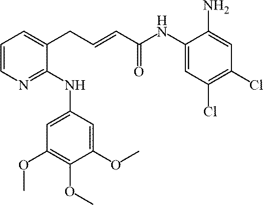 [SMILES: COc1cc(Nc2ncccc2C/C=C/C(=O)Nc2cc(Cl)c(Cl)cc2N)cc(OC)c1OC]

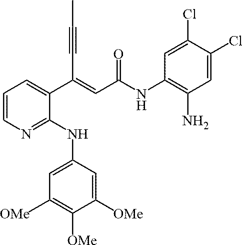 [SMILES: CC#C/C(=C\C(=O)Nc1cc(Cl)c(Cl)cc1N)c1cccnc1Nc1cc(OC)c(OC)c(OC)c1]

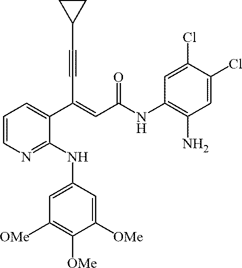 [SMILES: COc1cc(Nc2ncccc2/C(C#CC2CC2)=C/C(=O)Nc2cc(Cl)c(Cl)cc2N)cc(OC)c1OC]

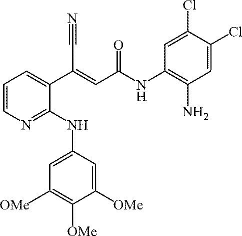 [SMILES: COc1cc(Nc2ncccc2/C(C#N)=C/C(=O)Nc2cc(Cl)c(Cl)cc2N)cc(OC)c1OC]

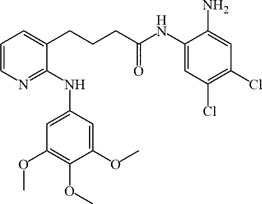 [SMILES: COc1cc(Nc2ncccc2CCCC(=O)Nc2cc(Cl)c(Cl)cc2N)cc(OC)c1OC]

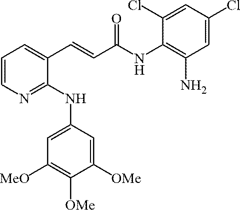 [SMILES: COc1cc(Nc2ncccc2/C=C/C(=O)Nc2c(N)cc(Cl)cc2Cl)cc(OC)c1OC]

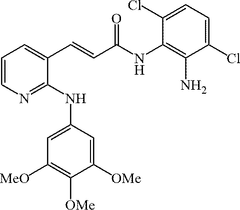 [SMILES: COc1cc(Nc2ncccc2/C=C/C(=O)Nc2c(Cl)ccc(Cl)c2N)cc(OC)c1OC]

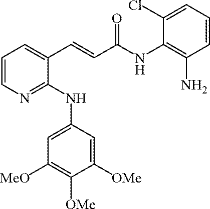 [SMILES: COc1cc(Nc2ncccc2/C=C/C(=O)Nc2c(N)cccc2Cl)cc(OC)c1OC]

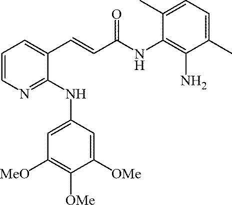 [SMILES: COc1cc(Nc2ncccc2/C=C/C(=O)Nc2c(C)ccc(C)c2N)cc(OC)c1OC]

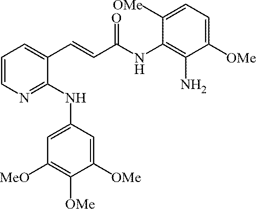 [SMILES: COc1ccc(OC)c(NC(=O)/C=C/c2cccnc2Nc2cc(OC)c(OC)c(OC)c2)c1N]

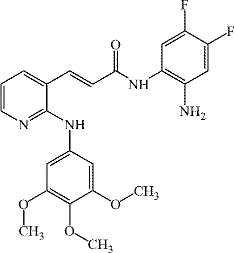 [SMILES: COc1cc(Nc2ncccc2/C=C/C(=O)Nc2cc(F)c(F)cc2N)cc(OC)c1OC]

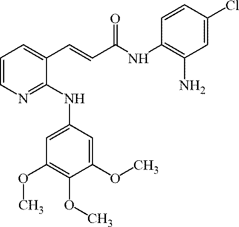 [SMILES: COc1cc(Nc2ncccc2/C=C/C(=O)Nc2ccc(Cl)cc2N)cc(OC)c1OC]

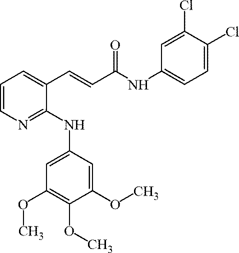 [SMILES: COc1cc(Nc2ncccc2/C=C/C(=O)Nc2ccc(Cl)c(Cl)c2)cc(OC)c1OC]

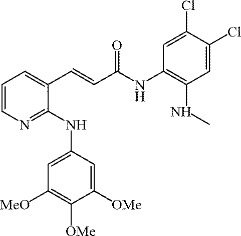 [SMILES: CNc1cc(Cl)c(Cl)cc1NC(=O)/C=C/c1cccnc1Nc1cc(OC)c(OC)c(OC)c1]

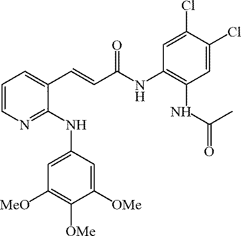 [SMILES: COc1cc(Nc2ncccc2/C=C/C(=O)Nc2cc(Cl)c(Cl)cc2NC(C)=O)cc(OC)c1OC]

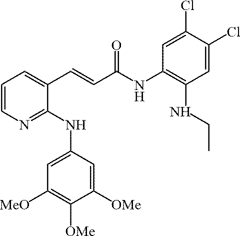 [SMILES: CCNc1cc(Cl)c(Cl)cc1NC(=O)/C=C/c1cccnc1Nc1cc(OC)c(OC)c(OC)c1]

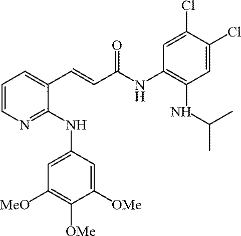 [SMILES: COc1cc(Nc2ncccc2/C=C/C(=O)Nc2cc(Cl)c(Cl)cc2NC(C)C)cc(OC)c1OC]

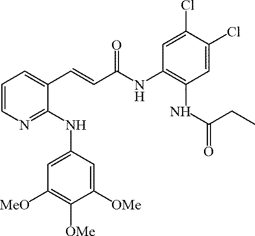 [SMILES: CCC(=O)Nc1cc(Cl)c(Cl)cc1NC(=O)/C=C/c1cccnc1Nc1cc(OC)c(OC)c(OC)c1]

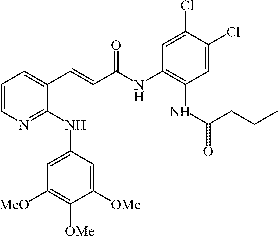 [SMILES: CCCC(=O)Nc1cc(Cl)c(Cl)cc1NC(=O)/C=C/c1cccnc1Nc1cc(OC)c(OC)c(OC)c1]

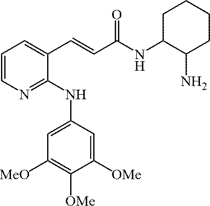 [SMILES: COc1cc(Nc2ncccc2/C=C/C(=O)NC2CCCCC2N)cc(OC)c1OC]

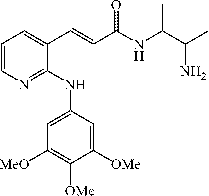 [SMILES: COc1cc(Nc2ncccc2/C=C/C(=O)NC(C)C(C)N)cc(OC)c1OC]

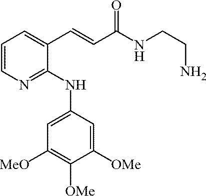 [SMILES: COc1cc(Nc2ncccc2/C=C/C(=O)NCCN)cc(OC)c1OC]

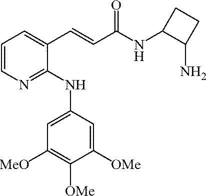 [SMILES: COc1cc(Nc2ncccc2/C=C/C(=O)NC2CCC2N)cc(OC)c1OC]

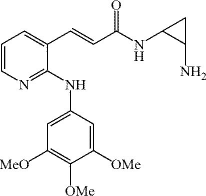 [SMILES: COc1cc(Nc2ncccc2/C=C/C(=O)NC2CC2N)cc(OC)c1OC]

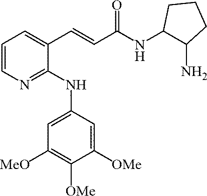 [SMILES: COc1cc(Nc2ncccc2/C=C/C(=O)NC2CCCC2N)cc(OC)c1OC]

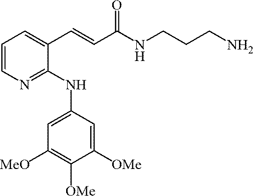 [SMILES: COc1cc(Nc2ncccc2/C=C/C(=O)NCCCN)cc(OC)c1OC]

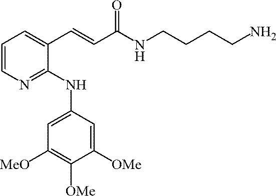 [SMILES: COc1cc(Nc2ncccc2/C=C/C(=O)NCCCCN)cc(OC)c1OC]

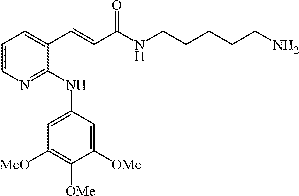 [SMILES: COc1cc(Nc2ncccc2/C=C/C(=O)NCCCCCN)cc(OC)c1OC]

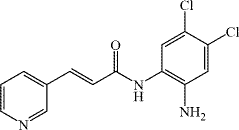 [SMILES: Nc1cc(Cl)c(Cl)cc1NC(=O)/C=C/c1cccnc1]

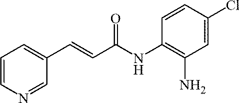 [SMILES: Nc1cc(Cl)ccc1NC(=O)/C=C/c1cccnc1]

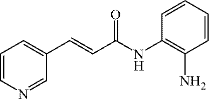 [SMILES: Nc1ccccc1NC(=O)/C=C/c1cccnc1]

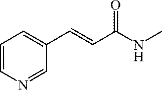

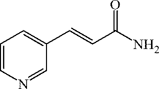 [SMILES: NC(=O)/C=C/c1cccnc1]

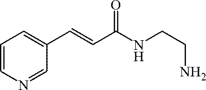 [SMILES: NCCNC(=O)/C=C/c1cccnc1]

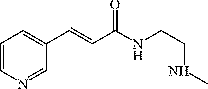

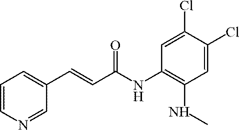 [SMILES: CNc1cc(Cl)c(Cl)cc1NC(=O)/C=C/c1cccnc1]

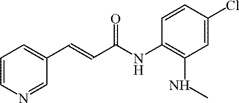 [SMILES: CNc1cc(Cl)ccc1NC(=O)/C=C/c1cccnc1]

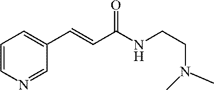 [SMILES: CN(C)CCNC(=O)/C=C/c1cccnc1]

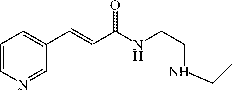 [SMILES: CCNCCNC(=O)/C=C/c1cccnc1]

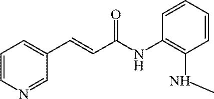 [SMILES: CNc1ccccc1NC(=O)/C=C/c1cccnc1]

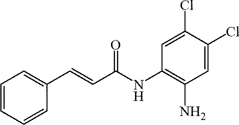 [SMILES: Nc1cc(Cl)c(Cl)cc1NC(=O)/C=C/c1ccccc1]

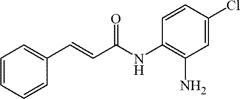 [SMILES: Nc1cc(Cl)ccc1NC(=O)/C=C/c1ccccc1]

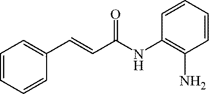 [SMILES: Nc1ccccc1NC(=O)/C=C/c1ccccc1]

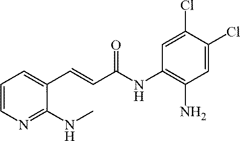 [SMILES: CNc1ncccc1/C=C/C(=O)Nc1cc(Cl)c(Cl)cc1N]

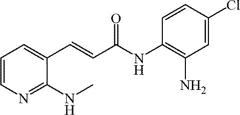 [SMILES: CNc1ncccc1/C=C/C(=O)Nc1ccc(Cl)cc1N]

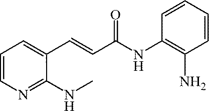 [SMILES: CNc1ncccc1/C=C/C(=O)Nc1ccccc1N]

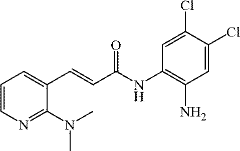 [SMILES: CN(C)c1ncccc1/C=C/C(=O)Nc1cc(Cl)c(Cl)cc1N]

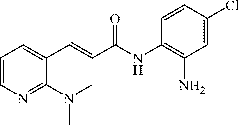 [SMILES: CN(C)c1ncccc1/C=C/C(=O)Nc1ccc(Cl)cc1N]

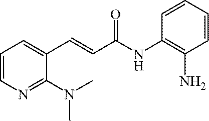 [SMILES: CN(C)c1ncccc1/C=C/C(=O)Nc1ccccc1N]

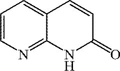 [SMILES: O=c1ccc2cccnc2[nH]1]

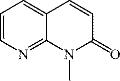

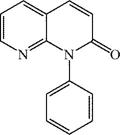 [SMILES: O=c1ccc2cccnc2n1-c1ccccc1]

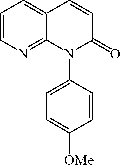 [SMILES: COc1ccc(-n2c(=O)ccc3cccnc32)cc1]

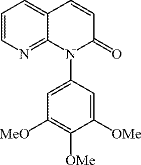 [SMILES: COc1cc(-n2c(=O)ccc3cccnc32)cc(OC)c1OC]

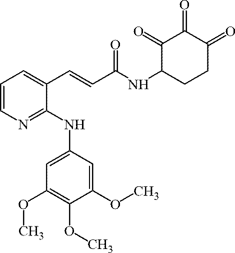 [SMILES: COc1cc(Nc2ncccc2/C=C/C(=O)NC2CCC(=O)C(=O)C2=O)cc(OC)c1OC]

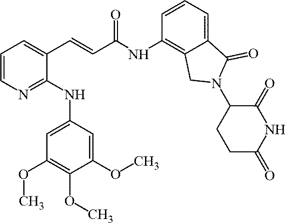 [SMILES: COc1cc(Nc2ncccc2/C=C/C(=O)Nc2cccc3c2CN(C2CCC(=O)NC2=O)C3=O)cc(OC)c1OC]

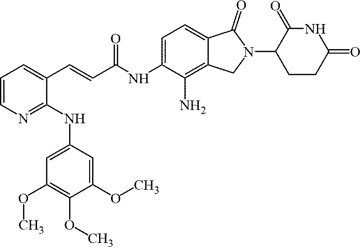 [SMILES: COc1cc(Nc2ncccc2/C=C/C(=O)Nc2ccc3c(c2N)CN(C2CCC(=O)NC2=O)C3=O)cc(OC)c1OC]

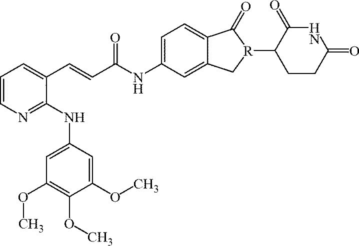

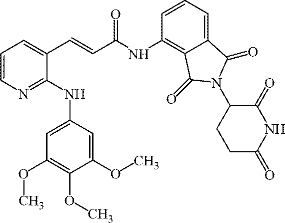 [SMILES: COc1cc(Nc2ncccc2/C=C/C(=O)Nc2cccc3c2C(=O)N(C2CCC(=O)NC2=O)C3=O)cc(OC)c1OC]

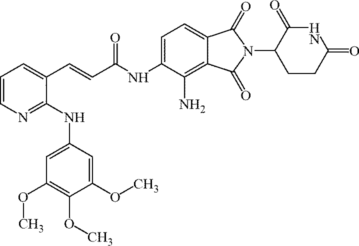 [SMILES: COc1cc(Nc2ncccc2/C=C/C(=O)Nc2ccc3c(c2N)C(=O)N(C2CCC(=O)NC2=O)C3=O)cc(OC)c1OC]

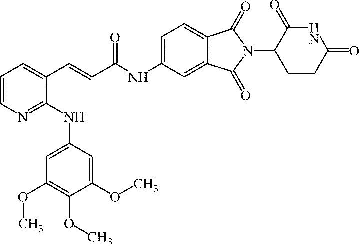 [SMILES: COc1cc(Nc2ncccc2/C=C/C(=O)Nc2ccc3c(c2)C(=O)N(C2CCC(=O)NC2=O)C3=O)cc(OC)c1OC]

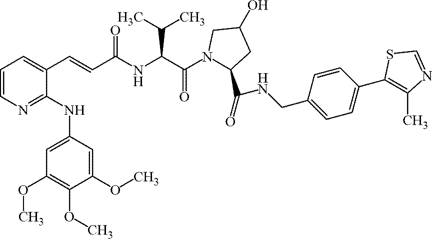 [SMILES: COc1cc(Nc2ncccc2/C=C/C(=O)N[C@H](C(=O)N2CC(O)C[C@H]2C(=O)NCc2ccc(-c3scnc3C)cc2)C(C)C)cc(OC)c1OC]

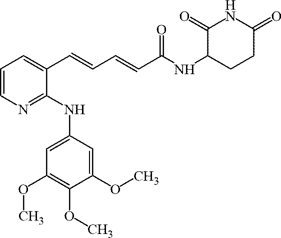 [SMILES: COc1cc(Nc2ncccc2/C=C/C=C/C(=O)NC2CCC(=O)NC2=O)cc(OC)c1OC]

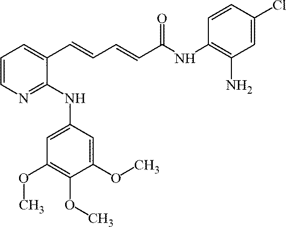 [SMILES: COc1cc(Nc2ncccc2/C=C/C=C/C(=O)Nc2ccc(Cl)cc2N)cc(OC)c1OC]

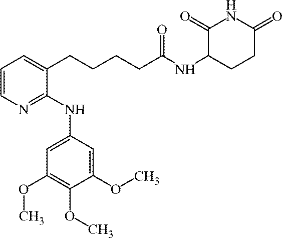 [SMILES: COc1cc(Nc2ncccc2CCCCC(=O)NC2CCC(=O)NC2=O)cc(OC)c1OC]

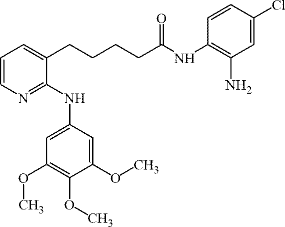 [SMILES: COc1cc(Nc2ncccc2CCCCC(=O)Nc2ccc(Cl)cc2N)cc(OC)c1OC]

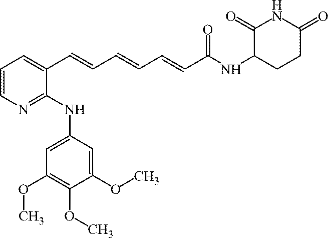 [SMILES: COc1cc(Nc2ncccc2/C=C/C=C/C=C/C(=O)NC2CCC(=O)NC2=O)cc(OC)c1OC]

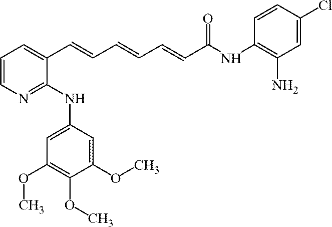 [SMILES: COc1cc(Nc2ncccc2/C=C/C=C/C=C/C(=O)Nc2ccc(Cl)cc2N)cc(OC)c1OC]

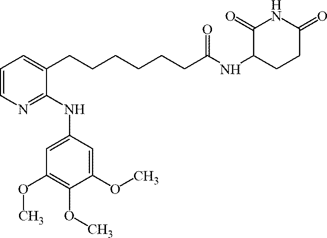 [SMILES: COc1cc(Nc2ncccc2CCCCCCC(=O)NC2CCC(=O)NC2=O)cc(OC)c1OC]

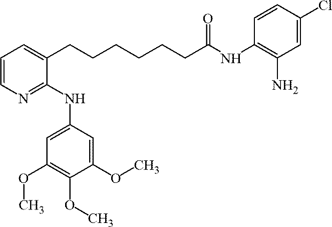 [SMILES: COc1cc(Nc2ncccc2CCCCCCC(=O)Nc2ccc(Cl)cc2N)cc(OC)c1OC]

### BRIEF DESCRIPTION

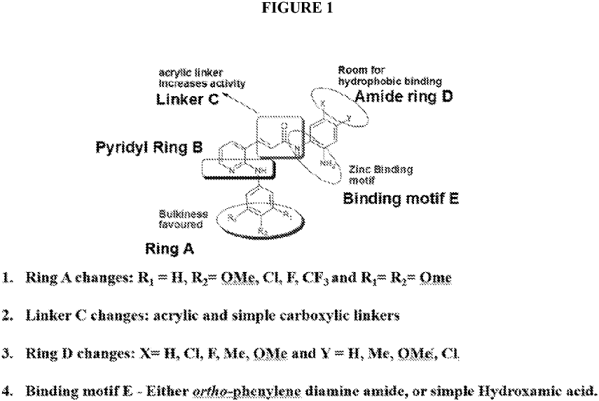

FIG. 1 shows general structures of compounds forming part of the library of compounds tested in Examples 1 and/or 2, and structure-activity relationship (SAR) results;

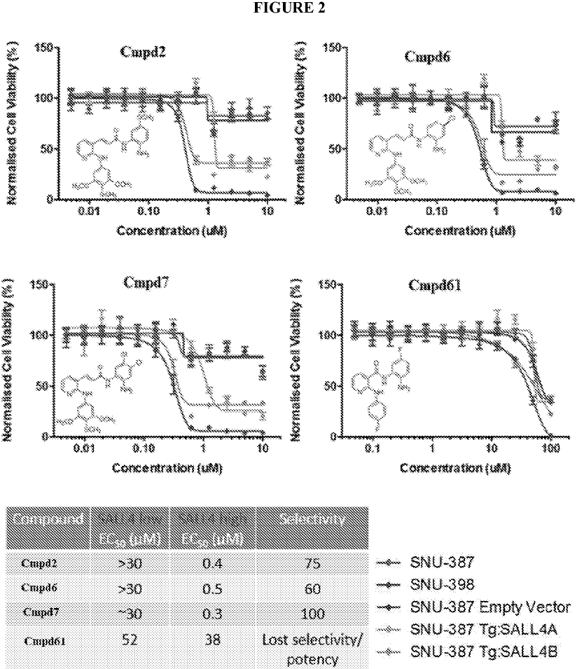

FIG. 2 shows results of SALL4 high/low cell viability phenotypic screening. In particular, FIG. 2 shows results of cell-based screening of 4 compounds of the library (i.e. Cmpd 2, Cmpd 6, Cmpd 7, and Cmpd 61) in SALL4 low and SALL4 high cells. As shown, each of Cmpd 2, Cmpd 6, and Cmpd 7 had excellent EC₅₀ and selectivity between SALL4 low and SALL4 high cells. Cmpd 61 (missing the acrylic linker), on the other hand, had low selectivity and potency;

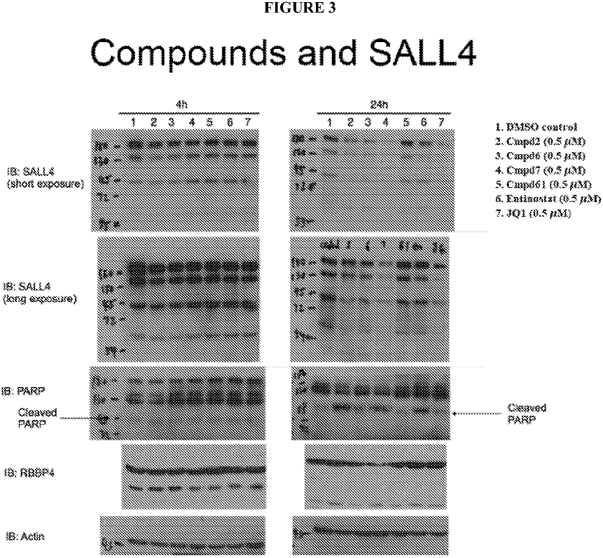

FIG. 3 shows Western blots showing SALL4 protein down regulation upon treatment of N-(2-aminophenyl)-prop-2-enamide derivatives. In particular, FIG. 3 shows results of testing with Cmpd 2, Cmpd 6, Cmpd 7, and Cmpd 61 for SALL4-related effects, also compared with entinostat and JQ1;

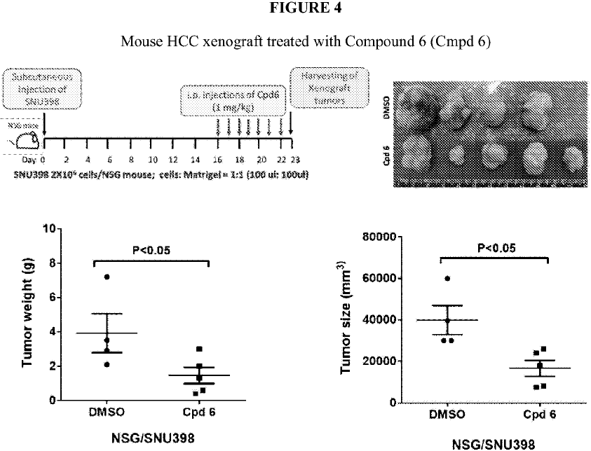

FIG. 4 shows results for in vivo transgenic mice experiments showing SALL4 high tumor responds to N-(2-aminophenyl)-prop-2-enamide derivatives. In particular, FIG. 4 shows results for in vivo xenotransplant testing for the Cmpd 6 compound. Mice treated with Compound 6 had significant (P<0.05) smaller xenografts both in size and weight;

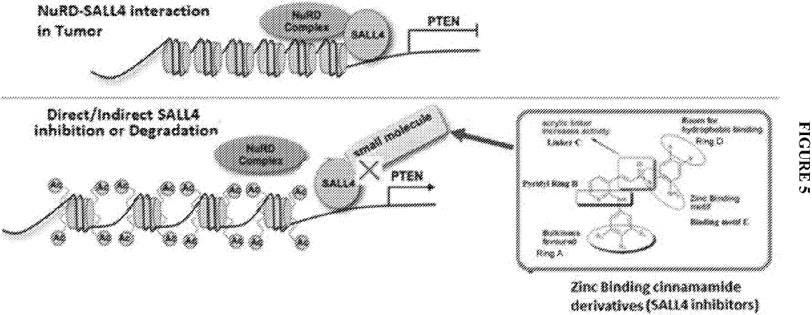

FIG. 5 shows a diagram of a proposed mechanism for SALL4 inhibition and restoration of PTEN in a tumor cell;

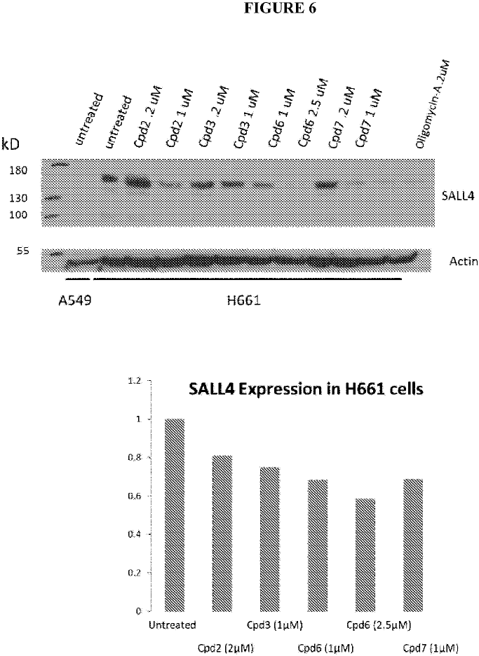

FIG. 6 shows results in which Compound 6 (Cmpd 6) and other derivatives were tested in two lung cancer cell lines, where A549 is SALL4-low, and H661 is SALL4-high. Cells were treated with compound 6 at 1 µM and 2.5 µM. Amounts for other cmpds are shown. Cells were harvested at 48 hrs and the cell lysates were subjected to western blot analysis and Q-PCR analysis for SALL4 protein and RNA level respectively. Upper panel, in the western blot analysis, it was found that Compound 6 could reduce SALL4 protein level at 2.5 µM. Lower Panel, using Q-PCR, it was shown that SALL4 RNA level was reduced by Compound 6 in H661 cells;

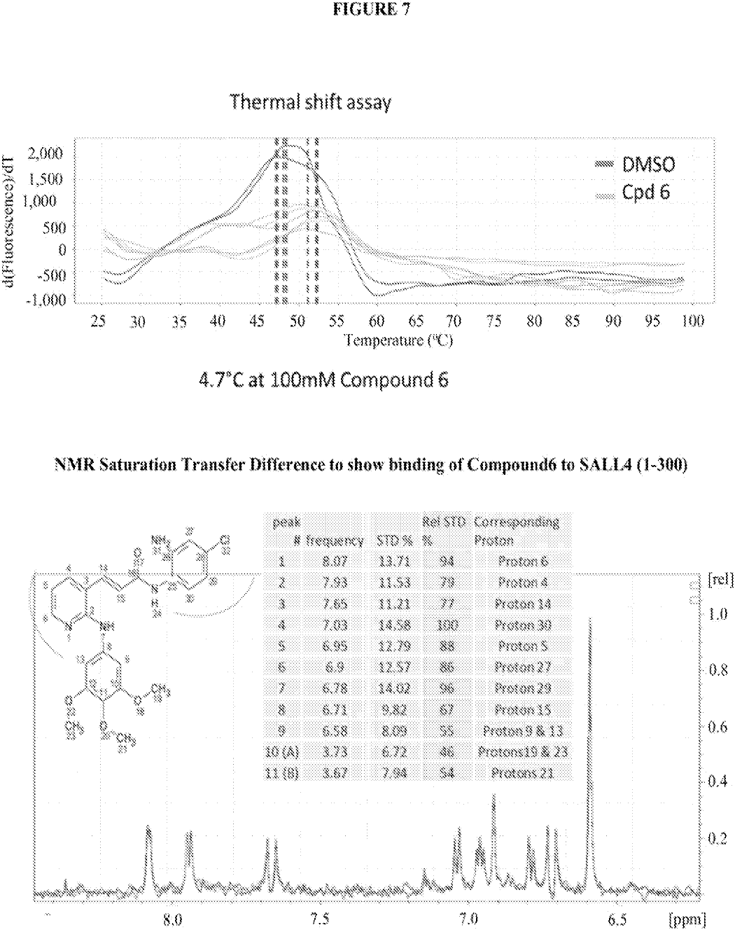

FIG. 7 shows results indicating that Compound 6 (Cmpd 6) binds to SALL4. Upper panel, A fluorescence-based binding assay named Thermal Shift Assay was used to assess the binding of compound 6 to SALL4 1-300 protein based on changes in unfolding transition. The result shows that incubation of SALL4 1-300 with 100 mM compound 6 resulted in a melting shift of 4.7° C., indicating compound 6 binds to SALL4. Lower panel, compound 6 binding to SALL4 was accessed by 1H NMR Experiments and Saturation Transfer Difference (STD) using ¹⁵N SALL4 1-300 and Compound 6. The samples were screened using Bruker BioSpin AVANCE II 600 MHz spectrometer equipped with a CPPTCI{ 19F} cryoprobe and SampleJet auto-sampler. Compound 6 weak binding event to SALL4 1-300 was identified based on the increase in transverse relaxation rate (R2) observed in the presence of SALL4 1-300 protein;

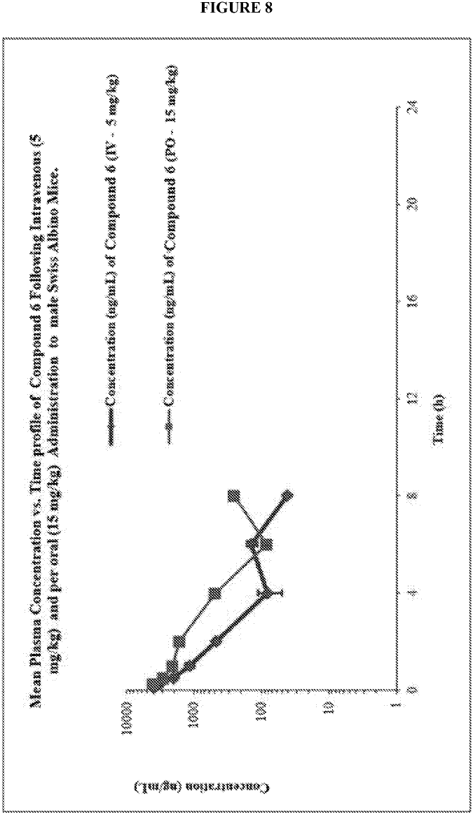

FIG. 8 shows results of a pharmacokinetic study of compound 6 (Cmpd 6). To understand the bioavailability of compound 6 (Cmpd 6), compound 6 were given orally (15 mg/kg), or via intravenous injection (5 mg/kg) to Swiss albino mice. Compound 6 was dissolved in water containing 3% DMSO and 10% hydroxyl propyl-b-cyclodextrin (EncapsinTM) for the i.v. dose, and in water containing 5% DMSO and 9.5% Encapsin for the oral dose. Blood samples were collected at 0.083, 0.25, 0.5, 1, 2, 4, 6, 8 and 24 hrs. The whole blood concentration was measured by LC-MS/MS. From the oral route, 90% of compound 6 was cleared at 2 hrs. For the intravenous route, 90% of compound 6 was cleared at 4 hrs;

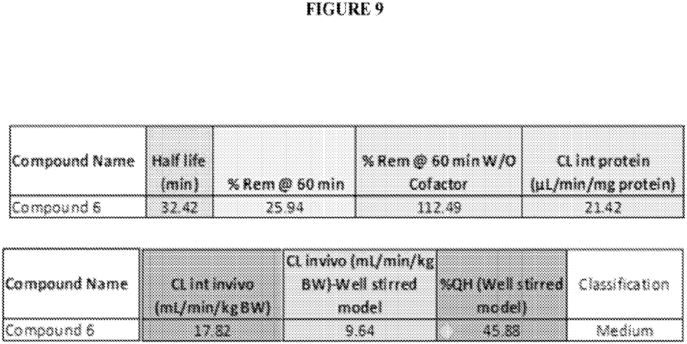

FIG. 9 shows results in which Compound 6 (Cmpd 6) was tested for metabolic stability using liver microsomes. IµM of compound 6 was incubated with 3.33 mg/ml liver microsomes and 2.5 mM NADPH for 0, 5, 10, 30 and 60 minutes. The mixture were subjected to LC-MS/MS to measure remaining compound after the incubation. The result shows that compound 6 has medium permeability status at 45.88 %QH;

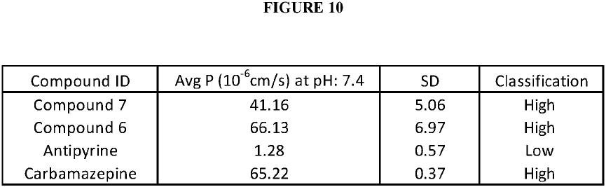

FIG. 10 shows results in which PAMPA permeability assay was performed on compound 6 and some comparators (Compound 7 (Cmpd 7), antipyrine, carbamazepine). 50 µM of Compound 6 prepared in pION buffer was added to bottom of UV plate. GIT-0 solution was added and the solution was incubated for 4 hours. The plate spectrum was read using spectrophotometer in scanning mode from 200 nm to 500 nm using PAMPA pION software. Result shows that compound 6 has high permeability in PAMPA assay; and

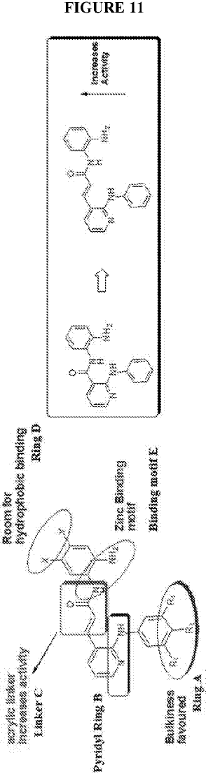

FIG. 11 shows key structure-activity findings from screening of the library as described in Example 1.

### DETAILED DESCRIPTION

Described herein are N-(2-aminophenyl)-prop-2-enamide derivatives, methods for the synthesis thereof, and uses thereof in the treatment of cancer. It will be appreciated that embodiments and examples are provided for illustrative purposes intended for those skilled in the art, and are not meant to be limiting in any way.

Embryonic factor SALL4 is mainly expressed in fetal and stem cells, and has been found to be aberrantly expressed in many cancers. Without wishing to be bound by theory, it is contemplated that SALL4 may bind to an epigenetic complex, NuRD, and may co-operatively repress promoter of tumor suppressor gene, PTEN, for example. Inhibition of SALL4, or the the SALL4-NuRD complex, may activate tumor suppressor pathways and/or may provide a druggable target for the treatment of cancers, especially lung and liver cancers. Inhibitors of SALL4, and/or anticancer agents active in SALL4-expressing cancer cells, are highly desirable.

To identify small molecules for inhibiting SALL4-RBBP4 binding, the present inventors sought to develop and investigate dual-motif derivatives as described in detail hereinbelow. Results detailed herein indicated that compounds have now been identified which may be selectively potent in SALL4 high cancer cell lines as compared to SALL4 low cells, and that this selectivity may be specifically and selectively due to SALL4-RBBP4 interaction. The derivatives tested showed growth inhibitory IC₅₀ values of 5-500 nM in SALL4 high liver and lung cancer cells with a selectivity of 50 to 400 times over the SALL4 low liver and cancer cells. A similar profile was observed for these derivatives on breast cancer cells (MBA-MD-231). These studies are described in further detail hereinbelow.

The inventors sought to develop small molecule compounds to inhibit SALL4 and/or activate tumor suppressor pathway(s), and designed a motif integrating certain features of an anilino (e.g. trimethoxyanilino) pharmacophore for tubulin inhibition, and certain features of a benzamide-type (i.e. N-(2-aminophenyl) amide) zinc binder/HDAC inhibitor. The anilino pharmacophore is somewhat related to structure in the compound E7010 which is a known tubulin polymerization inhibitor [6-9], and the motif may further include a portion having certain features which are somewhat related to structure in certain benzamide-type (i.e. N-(2-aminophenyl) amide) HDAC inhibitors (such as Chidamide and/or Entinostat).

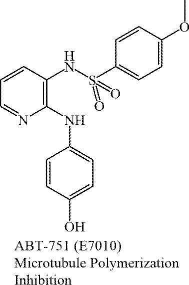

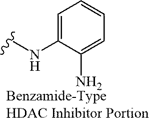

The initially designed series of compounds were as follows:

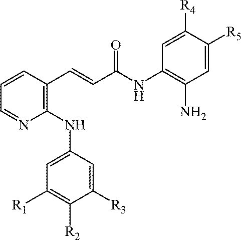

wherein:
R₁, R₂, and R₃ are each independently selected from H, F, Cl, CF₃, or —OCH₃; and
R₄ and R₅ are each independently selected from H, F, Cl, or —OCH₃.

Compounds from this collection form part of the compound library tested in Examples 1 and 2 below. Also as part of the library, simplified derivatives were also prepared as comparators, having the following formulas:

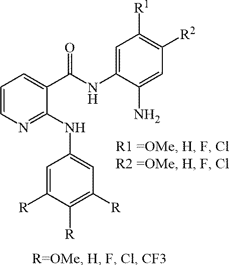

or

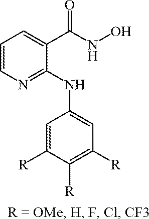

It was contemplated that the basic pyridyl amino part of E7010 may be important for RBBP4 binding, while the fragment having similarity to structure in certain benzamide-type (i.e. N-(2-aminophenyl) amide) HDAC inhibitors that is involved in the zinc binding part may be involved in inhibition of SALL4. Further, unlike other benzamide-type (i.e. N-(2-aminophenyl) amide) HDAC inhibitors, the presently developed compounds may have “2-anilinopyridyl” functionality rather than a benzyl substituted phenyl or pyridyl moiety, and may be further distinguished from other benzamide-type (i.e. N-(2-aminophenyl) amide) HDAC inhibitors in which there is no anilinopyridyl.

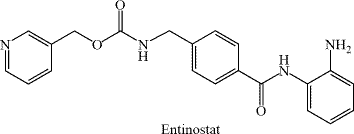

In testing and development of such compounds, it was discovered that such compounds as described herein exhibited SALL4 inhibition in SALL4 high cells invariably in lung, liver and breast cancers (see Examples below). In certain embodiments of compounds as described herein, HDAC inhibition/zinc binding was deliberately compromised for selectivity toward SALL4.

Results of testing of compounds from the compound library that was generated are described in detail in the Examples below. Overall, N-(2-aminophenyl)-prop-2-enamide derivatives having anticancer activity are identified herein. Extensive structure-activity studies were performed as described herein, and potent compounds and pharmacophores were identified. In certain embodiments, examples of N-(2-aminophenyl)-prop-2-enamide derivatives as described herein may provide potent anticancer and/or growth inhibitory activity, which may be selective for cells (such as lung cancer cells or liver cancer cells) having an elevated or high level of SALL4 expression as compared with cells having low SALL4 levels.

Accordingly, in certain embodiments, there is provided herein a compound of Formula I:

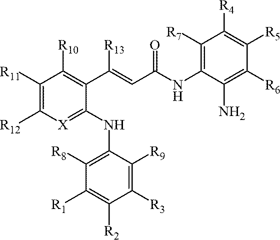

wherein
R₁ is H or —OCH₃;
R₂ is H, —OCH₃, —CF₃, or F;
R₃ is H or —OCH₃;
R₄ is H, Cl, —OCH₃, —CH₃, F, or —NH₂;
R₅ is H, Cl, —OCH₃, —CH₃, F, or —NH₂;
R₆ is H, Cl, —OCH₃, or —CH₃;
R₇ is H, Cl, —OCH₃, or —CH₃;
R₈ is H, —OCH₃, or —CH₃;
R₉ is H, —OCH₃, or —CH₃;
each of R₁₀, R₁₁, and R₁₂ is H, or R₁₀ and R₁₁ together with the carbon atoms to which they are attached form a 5 or 6-membered aryl or heteroaryl ring and R₁₂ is H, or R₁₁ and R₁₂ together with the carbon atoms to which they are attached form a 5 or 6-membered aryl or heteroaryl ring and R₁₀ is H;
R₁₃ is H, —C≡C—CH₃, —C≡C—C₃H₅, or —C≡N; and
X is N or CH.

In certain embodiments of any of the compound or compounds described herein, R₁ is —OCH₃. In certain embodiments of any of the compound or compounds described herein, R₂ is —OCH₃ or —CF₃. In certain embodiments of any of the compound or compounds described herein, R₃ is —OCH₃. In certain embodiments of any of the compound or compounds described herein, R₄ is H, Cl, —OCH₃, or —CH₃. In certain embodiments of any of the compound or compounds described herein, R₅ is H or Cl. In certain embodiments of any of the compound or compounds described herein, R₆ is H. In certain embodiments of any of the compound or compounds described herein, R₇ is H. In certain embodiments of any of the compound or compounds described herein, R₈ is H. In certain embodiments of any of the compound or compounds described herein, R₉ is H. In certain embodiments of any of the compound or compounds described herein, R₁₀, R₁₁, and R₁₂ are each H. In certain embodiments of any of the compound or compounds described herein, R₁₃ is H. In certain embodiments of any of the compound or compounds described herein, X is N.

In certain embodiments, there is provided herein a compound which is:

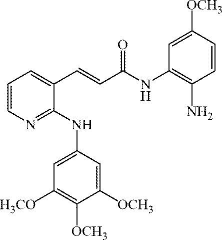 [SMILES: COc1ccc(N)c(NC(=O)/C=C/c2cccnc2Nc2cc(OC)c(OC)c(OC)c2)c1]

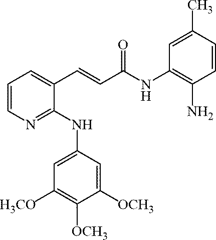 [SMILES: COc1cc(Nc2ncccc2/C=C/C(=O)Nc2cc(C)ccc2N)cc(OC)c1OC]

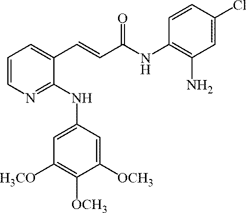 [SMILES: COc1cc(Nc2ncccc2/C=C/C(=O)Nc2ccc(Cl)cc2N)cc(OC)c1OC]

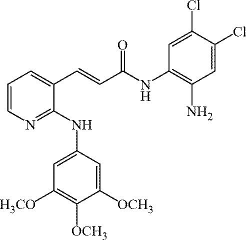 [SMILES: COc1cc(Nc2ncccc2/C=C/C(=O)Nc2cc(Cl)c(Cl)cc2N)cc(OC)c1OC]

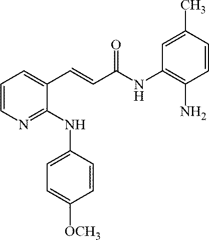 [SMILES: COc1ccc(Nc2ncccc2/C=C/C(=O)Nc2cc(C)ccc2N)cc1]

or

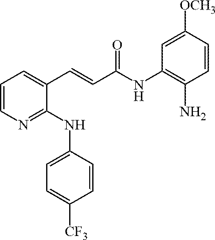 [SMILES: COc1ccc(N)c(NC(=O)/C=C/c2cccnc2Nc2ccc(C(F)(F)F)cc2)c1]

In an embodiment, there is provided herein a compound which is:

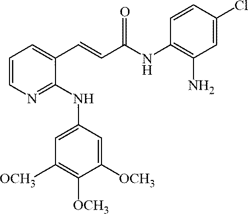

In another embodiment, there is provided herein a composition comprising any one or more of the compounds described herein, and a pharmaceutically acceptable carrier, excipient, or diluent. In certain embodiments, a pharmaceutically acceptable carrier, diluent, or excipient may include any suitable carrier, diluent, or excipient known to the person of skill in the art. Examples of pharmaceutically acceptable excipients may include, but are not limited to, cellulose derivatives, sucrose, and starch. The person of skill in the art will recognize that pharmaceutically acceptable excipients may include suitable fillers, binders, lubricants, buffers, glidants, and disentegrants known in the art (see, for example, Remington: The Science and Practice of Pharmacy (2006)). Examples of pharmaceutically acceptable carriers, diluents, and excipients may be found in, for example, Remington’s Pharmaceutical Sciences (2000 — 20th edition) and in the United States Pharmacopeia: The National Formulary (USP 24 NF19) published in 1999.

In certain embodiments, there is provided herein a prodrug, salt, solvate, crystalline form, conjugate, radiolabelled or isotopically labelled form, or other such derivative of any of the compounds or compositions as described herein. The skilled person having regard to the teachings herein will understand, for example, that one or more protonated, deprotonated, or salt forms (such as a pharmaceutically acceptable salt form) of a compound as described herein may be obtained, and that all such forms are also contemplated herein.

In certain embodiments, one or more compounds as described herein may be formulated as an oral dosage form. In certain embodiments, one or more compounds as described herein may be for oral administration to a subject in need thereof.

In certain embodiments, a compound or composition as described herein may be for use in the treatment of cancer in a cell or a subject in need thereof. In another embodiment, there is provided herein a use of a compound or a composition as described herein for the treatment of cancer in a cell or a subject in need thereof. In still another embodiment, there is provided herein a use of a compound or composition as described herein in the manufacture of a medicament for use in the treatment of cancer in a cell or a subject in need thereof. In certain embodiments, the treatment may be an in vitro treatment, an ex vivo treatment, or an in vivo treatment, for example.

In certain embodiments, the cancer may be a SALL4-expressing cancer. In certain further embodiments, the cancer may be a SALL4-expressing cancer having a high level of SALL4 expression. In certain embodiments, SALL4 high may be measured by protein expression using, for example, Western blot and/or RNA expression such as by real time PCR, for example. As reference, in certain embodiments, examples of SALL4 “high” cells may include, for example, SNU398 cell line, whereas SALL4 “low” cells may include, for example, SNU387 cell line. In certain embodiments, the cancer may be lung cancer, liver cancer, or breast cancer. By way of example, in certain embodiments the cancer may be NSCLC cancer, cervical cancer, or germ cell cancer. In certain embodiments, a cancer or cancer cell having a high level of SALL4 expression may comprise a cancer or cancer cell in which a level of SALL4 expression is increased relative to a healthy control, or relative to a healthy or control comparator cell, for example.

In still another embodiment, there is provided herein an in vitro, ex vivo, or in vivo method for treating cancer in a cell or subject in need thereof, said method comprising:

administering a compound or a composition as described herein to the cell or subject.

In certain embodiments, the cancer may be a SALL4-expressing cancer. In certain embodiments, the cancer may be a SALL4-expressing cancer having a high level of SALL4 expression. In certain embodiments, the cancer may be lung cancer, liver cancer, or breast cancer. By way of example, in certain embodiments, the cancer may be NSCLC cancer, cervical cancer, or germ cell cancer.

In still another embodiment, there is provided herein a method for preparing an N-(2-aminophenyl)-prop-2-enamide derivative, said method comprising:
reacting a compound of formula 1 with a compound of formula 2 to form a compound of formula 3:

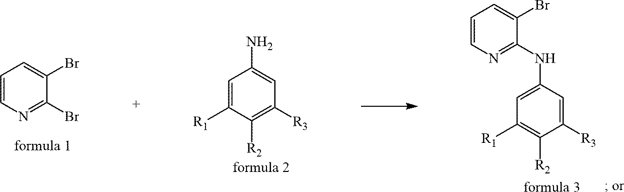

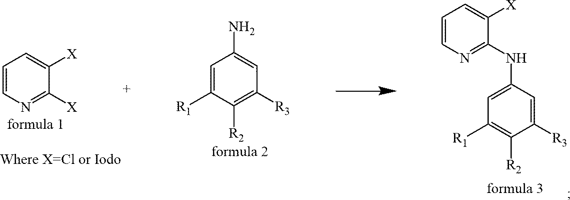

reacting the compound of formula 3 with a compound of formula 4, and deprotecting, to form a compound of formula 5:

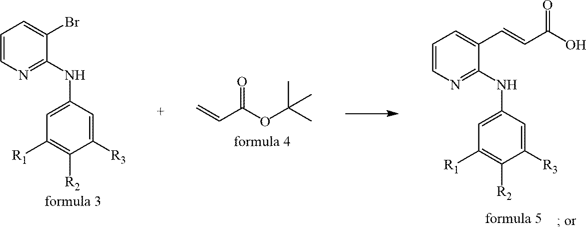

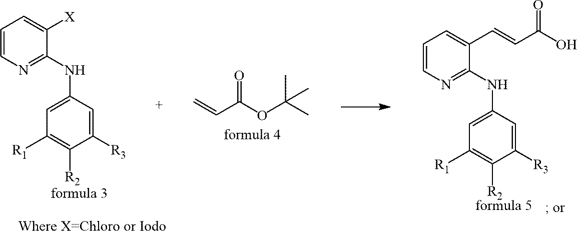

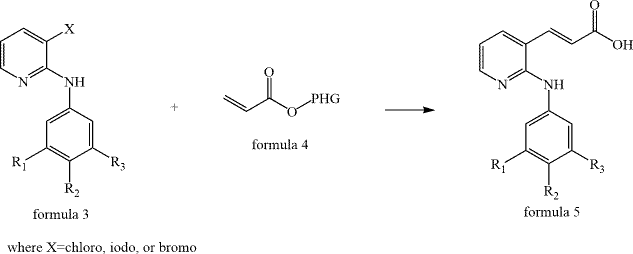

where PG represents any suitable protecting group (see, for example, Greene’s Protective Groups in Organic Synthesis, Fourth Edition, 2006, John Wiley & Sons, herein incorproated by reference in it’s entirety) known to the person of skill in the art having regard to the teachings herein (such as, but not limited to, an alkyl ester such methyl ester, ethyl ester, or t-butyl ester), and deprotecting includes removal of the PG protecting group to form a compound of formula 5 (deprotecting conditions may be selected based on the protecting group used - examples may include, for example, an acidic or basic hydrolysis to yield formula 5 - examples of deprotecting conditions may be found, for example, in Greene’s Protective Groups in Organic Synthesis, Fourth Edition, 2006, John Wiley & Sons, herein incorproated by reference in it’s entirety); and
reacting a compound of formula 5 with a compound of formula 6 to form an N-(2-aminophenyl)-prop-2-enamide derivative of formula 7:

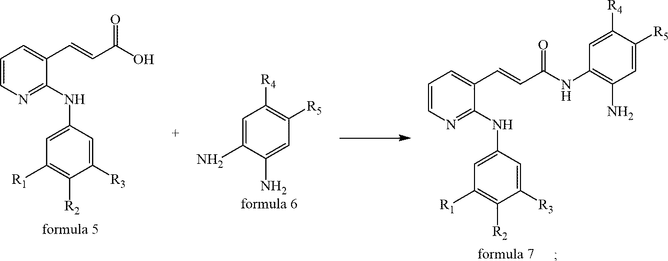

wherein
R₁, R₂, and R₃ are each independently selected from H, —OCH₃, —CF₃, Cl, or F;
R₄ is H, Cl, —OCH₃, —CH₃, or F; and
R₅ is H, Cl, —OCH₃, —CH₃, or F.

In an alternative embodiment of the above method, the compound of formula 5 may instead be prepared by reacting a compound of formula 3b with a compound of formula 2:

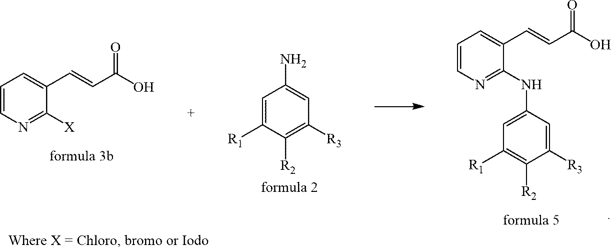

In another embodiment of any of the method or methods above, the compound of formula 1 may be reacted with the compound of formula 2 in NaOBu-t, Pd(OAc)₂, PPh₃, and xylene.

In still another embodiment of any of the method or methods above, the compound of formula 3 may be reacted with the compound of formula 4 in Pd₂(dba)₃, DMF, and DIPEA.

In yet another embodiment of any of the method or methods above, deprotection to form a compound of formula 5 may be performed with TFA in DCM.

In another embodiment of any of the method or methods above, the compound of formula 5 may be reacted with the compound of formula 6 in BOP, Et₃N, and MeCN.

In another embodiment, there is provided herein a compound produced by any of the above method or methods. In certain embodiments, the compound of formula 7 may be:

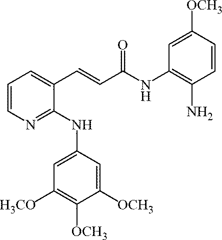 [SMILES: COc1ccc(N)c(NC(=O)/C=C/c2cccnc2Nc2cc(OC)c(OC)c(OC)c2)c1]

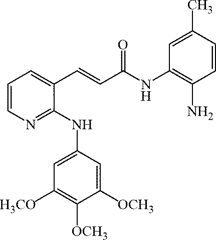 [SMILES: COc1cc(Nc2ncccc2/C=C/C(=O)Nc2cc(C)ccc2N)cc(OC)c1OC]

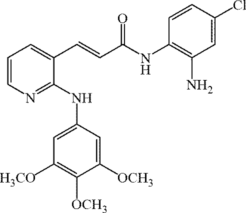 [SMILES: COc1cc(Nc2ncccc2/C=C/C(=O)Nc2ccc(Cl)cc2N)cc(OC)c1OC]

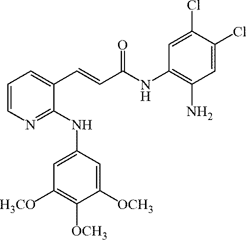 [SMILES: COc1cc(Nc2ncccc2/C=C/C(=O)Nc2cc(Cl)c(Cl)cc2N)cc(OC)c1OC]

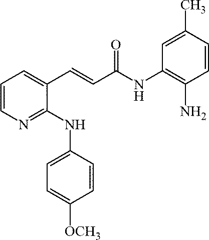 [SMILES: COc1ccc(Nc2ncccc2/C=C/C(=O)Nc2cc(C)ccc2N)cc1]

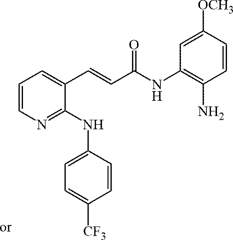

### Example 1 - Screening and Testing of Compound Library for SAR

Approximately 80 compounds were synthesised as represented in synthetic Schemes 1 and 2 below (referred to herein as the compound library), and tested against breast cancer SALL4 high and SALL4/low liver and lung cell lines.

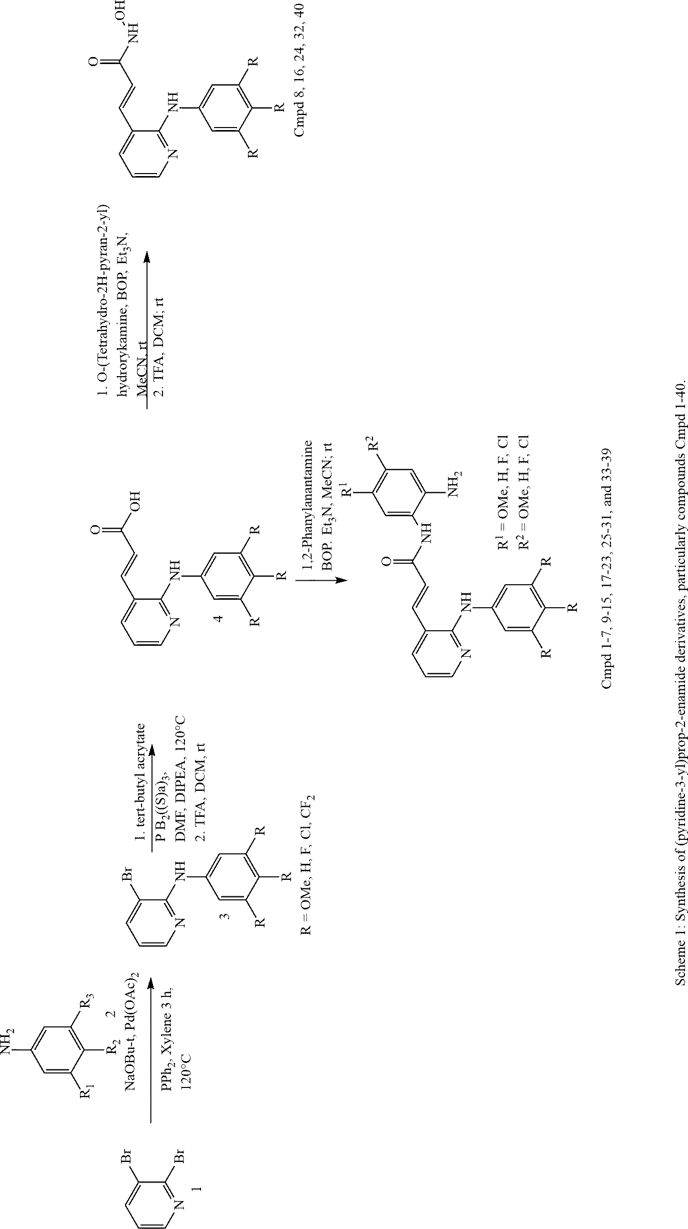

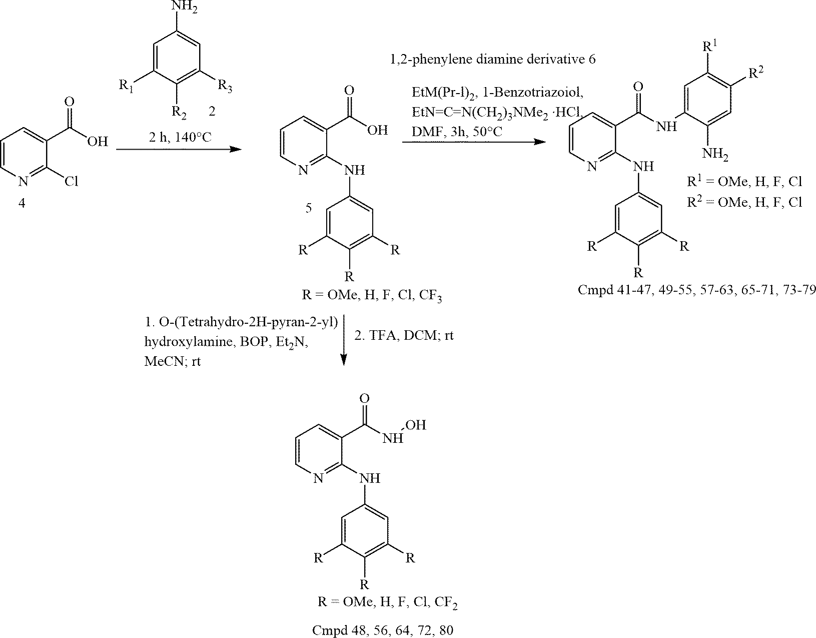

Some of the compounds of the library are summarized in FIG. 1.

Example syntheses were as follows:

Synthesis 3-Bromo-N-(3,4,5-trimethoxypheny1)pyridine-2-amine (an example of Formula 3, labelled as “3”’ in Scheme 1 above): In a 5-ml screw-capped vial, palladium acetate (0.14 mg, MW-225, 0.62 mmol), 4,5-bis(diphenylphosphino)-9,9-dimethylxanthene (Xantphos) (0.73 g, MW-579, 1.26 mmol), cesium carbonate (8.25 g, MW-326, 25.2 mmol), 2,3-dibromopyridine (3.0 g, MW-237, 12.6 mmol) and degassed toluene (30 ml) were mixed, and to this, trimethoxyaniline (2.31 g, MW-183, 12.6 mmol) was added. Then, nitrogen gas was enclosed in the vial, and the vial was stoppered tightly and the mixture was stirred with heating at an external temperature of 115° C. for 1.5 hours. After completion of the reaction, the reaction solution was cooled to room temperature, toluene (150 ml) and water (150 ml) were added thereto, and the insoluble was filtered off through Celite. The filtrate was washed with toluene (300 ml), and then the obtained organic layer was washed with saturated brine (250 ml), dried over anhydrous sodium sulfate and concentrated under reduced pressure. To the residue, a mixture (500 ml) of hexane/ethyl acetate (5/1) was added. The mixture was stirred at room temperature for 10 minutes, and then the insoluble was filtered off. The filtrate was concentrated under reduced pressure, and dried under reduced pressure at 50℃, to yield the title compound (3.4 g, MW -339) as a pale yellow solid (yield 80%). ¹H-NMR (CDCl₃, 400 MHz) δ (ppm): 8.17(dd, 1H, J= 8.0, 4.0 Hz, 1H), 7.75 (dd, 1H, J= 8.0, 4.0 Hz), 6.91 (brs, 1H), 6.91 (s, 2H), 6.65 (m, 1H), 3.89 (s, 6H), 3.83 (s, 3H). ¹³ C-NMR (CDCl₃, 400 MHz) δ (ppm): 153.35, 152.02, 146.49, 140.25, 135.72, 134.05, 115.51, 106.30, 98.34, 60.94, 56.13.

Synthesis of (2E-3-[2-3,4,5-trimethoxyanilino)pyridin-3-yl]prop-2-enoic acid t-butyl ester (an example of Formula 5a, a precursor of Formula 5): In a 100 mL flask, a mixture of Formula 3 (880 mg, 5 mmol), tert-butylacrylate (4 mL, 27.5 mmol), Diisopropyl ethylamine(4 mL, 23 mmol), tri-o-tolylphosphine (0.95 g, 3 mmol), Pd₂(dba)₃ (0.375 g, 0.4 mmol) in anhydrous DMF (20 mL) was stirred at 120° C. (preheated oil bath) for 2 h under nitrogen. After removal of DMF, the crude residue was purified using column chromatography (0 to 1%) DCM:MeOH) to afford 0.95 g of product. ¹H-NMR (CDCl₃, 400 MHz) () (ppm): 8.25 (dd, 111, J= 8.0, 4.0 Hz), 7.70 (m, 2H), 6.82 (m, 3H), 6.43 (s, 1H), 6.37 (d, 1H, J= 12 Hz), 3.86 (s, 6H), 3.83 (s, 3H), 1.54 (s, 9H). ¹³C-NMR (CDCl₃, 400 MHz) δ (ppm): 165.85, 153.36, 153.31, 149.34, 137.48, 136.19, 136.17, 133.98, 123.26, 116.85, 115.52, 98.54, 81.05, 60.91, 56.11, 28.16.

Synthesis of (2E)-3-[2-(3,4,5-trimethoxyanilino)pyridin-3-yl]prop-2-enoic acid (an example of Formula 5, labelled as “4” in Scheme 1 above): Ester Formula 5a (0.9 g, mmol) dissolved in 40% TFA in dichloromethane (50 mL) and the solution was stirred at room temperature overnight. The solvent was removed under vacuum with acetonitrile (3x30 mL) and stored under high vacuum for 12h. The solid residue Formula 5 (0.72 g, 93%) was used for coupling with amines as such without further purification. ¹H-NMR (CDCl₃, 400 MHz) δ (ppm): 9.03(hrs, 1H), 8.09 (m, 2H), 7.91 (d, 1H, J= 8.0 Hz), 6.88 (brs, 3H), 6.50 (m, 1H), 3.75 (s, 6H), 3.66 (s, 3H).

Synthesis of Compound 6, (2E)-N-(2-amino-4-chloropheny])-3-[2-(3, 4, 5-trimethoxy anilino) pyridin-3-yl]prop-2-enamide (Cmpd 6 as shown above): In a 100 mL RB flask acid Formula 5 (MW-330, 1 g, 3.03 mmol) was added DMF (15 mL), HATU (MW-380, 1.4 g, 3.6 mmol), diisopropyl ethylamine (129.25, 2.10 ml, 12.5 mmol) and stirred for 15 min. Then the corresponding amine, chlorophenylenediamine (MW-142, 0.4 g, 2.83 mmol) was added to the reaction mixture and the solution was stirred at 22° C. for 21 h. Then H₂O (20 mL) was added; the resulting precipitate was isolated by vacuum filtration and purified by silica gel chromatography (97:3:: CH₂Cl₂/ MeOH) to afford the desired product as a pale yellow solid. Mol Wt-454. ¹H-NMR (DMSO-D₆, 400 MHz) δ (ppm): 9.41 (brs, 1H), 8.54 (s, 1H), 8.19 (d, 1H, J= 4 Hz), 7.87 (m, 2H), 7.41 (d, 1H, J= 8 Hz), 7.00 (s, 2H), 6.84 (m, 3 Hz), 6.61 (dd, 1H,J= 8, 2.4 Hz), 5.30 (brs, 2H), 3.74 (s, 6H), 3.62 (s, 3H).

The same sequence of procedures was adopted for the synthesis of the compounds 1-40. For the compound 8, 16, 24, 32 and 40 there was an alternative step involved subsequent to the synthesis of Formula 5, which is TFA-mediated deprotection. Similar coupling and amide coupling was adopted for the synthesis of compounds 41-80 as shown in Scheme 2.

Compound 7: ¹H-NMR (DMSO-D₆,400 MHz) δ (ppm): 9.44 (brs, 1H), 8.54 (s, 1H), 8.20 (d, 1H, J = 4 Hz), 7.89 (m, 2H), 7.77 (s, 1H), 6.99 (s, 2H), 6.95 (s, 1 Hz), 6.91 (dd, 1H, J = 8 Hz), 6.84, (d, 1H, J = 16 Hz), 5.48 (brs, 2H), 3.74 (s, 6H), 3.62 (s, 3H). ¹³C-NMR (DMSO-D₆, 400 MHz) δ (ppm): 163.82, 153.41, 152.43, 148.70, 141.49, 137.43, 135.35, 132.31, 126.82, 124.88, 123.62, 123.43, 117.21, 116.10, 115.78, 115.39, 114.06, 98.19, 60.09, 55.69.

Compound 5: ¹H-NMR (DMSO-D₆,400 MHz) δ (ppm): 9.35 (brs, 1H), 8.52 (s, 1H), 8.19 (d, 1H, J = 4 Hz), 7.85 (m, 2H), 7.32 (dd, 1H, J = 8 Hz), 6.99 (s, 2H), 6.89 (m, 1 H), 6.81 (d, 1H, J = 12 Hz), 6.55 (dd, 1H, J = 8, 4 Hz), 6.36 (m, 1H), 5.28 (brs, 2H), 3.74 (s, 6H), 3.62 (s, 3H).

A set of compounds from the library were identified in this testing as being active against the tested cell lines with IC₅₀ in the sub-micromolar range, and showed a clear structure activity relationship of these molecules which was mostly consistent with the cancer cells screened. Two particularly potent compounds (Cmpd 6 and Cmpd 7) from the library showed sub-micromolar activity only in high SALL4 cells whereas the low SALL4 cell inhibition was observed at high micromolar concentration (see FIG. 2). That is, the selectivity was up to about 100 fold in high SALL4 cells compared to low SALL4 cells.

Key structure-activity findings from this screening of the library may be summarized as follows (see FIG. 11 for graphical representation):
a binding motif related to that of certain benzamide-type (i.e. N-(2-aminophenyl)prop-2-enamide) HDAC inhibitors was more active than a hydroxamic acid moiety at the zinc binding region E of the molecule;
an acrylic moiety at the linker part C was more effective than the short-chain, linker-less derivatives;
substitution of hydrophobic groups at the diamine ring D was favored. 4-chloro and 3,4-dichloro substitution improved potency of the series more than other substituents such as fluoro, methyl, or methoxy;
steric bulk, such as 3, 4, 5-trimethoxy groups, at aniline phenyl ring A were favored over other substitutions such as chloro, fluoro, trifluoromethyl or monomethoxy. Electronegative fluoro or trifluoromethoxy substitutions at the aniline phenyl ring A may lead to reduction of activity; and
2-Anilinopyridyl Ring B remained as a feature of the chemotype. Traditional HDAC inhibitors have been mostly para-substituted, allowing zinc binding accompanied by reduced steric hinderance. In the present compounds, HDAC inhibition/zinc binding was compromised by sterically bulky substitution at the ortho position. However, it was found that this steric factor may have facilitated SALL4 selectivity, as described in detail herein.

Apart from the cell-based activity, results indicated that derivatives from the library down-regulated the SALL4 protein expression of H661 at concentrations of 1µM. CBL-B RNA and PTEN RNA expressions were up-regulated by certain derivatives of the library while c-Myc RNA expression was down-regulated by some derivatives. The potent members of this class of compounds were 125x more potent than the clinical lead entinostat, and showed high selectivity for SALL4 high cancer cells. They possessed very good permeability as well. Compounds and results are described in further detail below (see, in particular, Table 2).

### Example 2 - Screening and Testing of Compound Library

Screening and testing of the compound library was performed. Various compounds of the library were tested in in vitro and in vivo assays described below to further investigate the anticancer properties, and various other properties, of these compounds.

### Materials and Methods

### Sall4 High/Low Phenotypic Screen

SNU-387 empty vector, Tg:SALL4A, and Tg:SALL4B expressing isogenic cell lines were generated by transducing WT SNU-387 cells with empty vector, SALL4A or SALL4B FUW-Luc-mCh-puro lentiviral constructs¹. Cells were plated in 50 µl of RPMI culture media in 384-well white flat-bottom plates (Coming) and incubated at 37° C. in a humidified atmosphere of 5% CO2 overnight. Cell numbers per well were 1500 for SNU-398, and 750 for SNU-387 and SNU-387 isogenic lines. After overnight incubation, varying concentrations of compounds 1-80 were added to cells with multichannel electronic pipettes (Rainin). Cells were then incubated for 72 hrs at 37° C. in a humidified atmosphere of 5% CO2 before 10 µl of CellTiter-Glo reagent was added to the wells with the MultiFlo Microplate Dispenser (BioTek). Cells were incubated at room temperature for a mininuun of 10 minutes after which luminescence readings were recorded by an Infinite M1000 Microplate Reader (Tecan). Key results are shown in FIG. 2 and in Table 2.

### Sall4 Protein Down Regulation/Western Blots

The SNU398 cells were incubated with the compounds (Cmpd1 to Cmpd80) for indicated time in the figure. Cells were then harvested by cell scraper and washed with PBS. The collected pellets were lysed with RIPA buffer (50 mM Tris, 150 mM NaCl, 1% TritotiX-100, 0.5% sodium deoxycholate, and 0.1% SDS) supplemented with protease inhibitor cocktail. The extracted protein lysates were denatured with 4X SDS sample buffer (200 mM Tris-HCl pH 6.8, 8% SDS, 40% glycerol, 4% β-mercaptoethanol, 50 mM EDTA, 0.08% bromophenol blue) at 99° C. for 5 minutes. Equal amount of protein were subjected to electrophoresis in 8% SDS-PAGE gel, and then transferred to PVDF membrane. After blocking in Blocking One (Nacalai Tesque), the membrane was probed with primary antibodies to SALL4 (Santa Cruz Biotechnology, sc-101147) and β-Actin (Santa Cruz Biotechnology, sc-47778) overnight at 4° C. After washing with TBS-T, membrane was incubated with secondary HRP-conjugated antibody to mouse for 1 hour (Santa Cruz Biotechnology, sc-2005). Luminata™ western HRP substrate (Millipore) was applied to the membrane for visualization. Key results are shown in FIG. 3.

### Results and Discussion

Of the compounds from the library that were tested in these studies, the following compounds were identified as being the most active (i.e. these compounds had high activity, with IC₅₀ of ~0.15 to 1 µM):

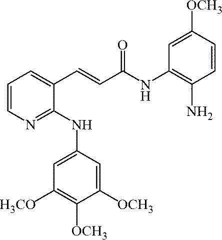 [SMILES: COc1ccc(N)c(NC(=O)/C=C/c2cccnc2Nc2cc(OC)c(OC)c(OC)c2)c1]

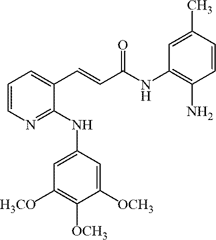 [SMILES: COc1cc(Nc2ncccc2/C=C/C(=O)Nc2cc(C)ccc2N)cc(OC)c1OC]

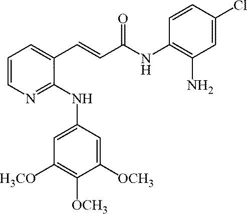 [SMILES: COc1cc(Nc2ncccc2/C=C/C(=O)Nc2ccc(Cl)cc2N)cc(OC)c1OC]

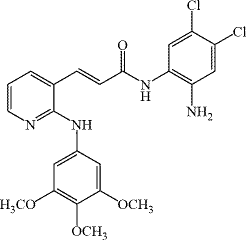 [SMILES: COc1cc(Nc2ncccc2/C=C/C(=O)Nc2cc(Cl)c(Cl)cc2N)cc(OC)c1OC]

the following compound was identified as being moderately active (i.e. about 10 fold less potent, IC₅₀ of ~2 µM range):

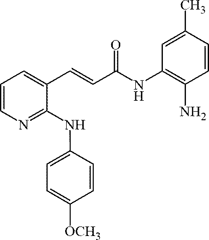 [SMILES: COc1ccc(Nc2ncccc2/C=C/C(=O)Nc2cc(C)ccc2N)cc1]

the following compound was identified as having lower activity (i.e. about 100 fold less potent, IC₅₀ of ~25 µM range):

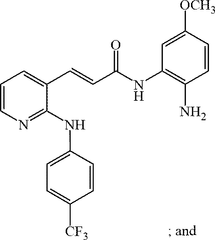

the following compounds were identified as being inactive:

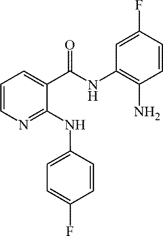 [SMILES: Nc1ccc(F)cc1NC(=O)c1cccnc1Nc1ccc(F)cc1]

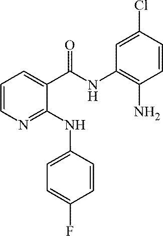 [SMILES: Nc1ccc(Cl)cc1NC(=O)c1cccnc1Nc1ccc(F)cc1]

This testing was based on SALL4 high/low phenotypic screen as described herein. IC₅₀ values noted for the compounds/groups above are based on ICso in high SALL4 SNU398 cells.

FIG. 2 shows results of cell-based screening of 4 compounds of the library (i.e. Cmpd 2, Cmpd 6, Cmpd 7, and Cmpd 61) in SALL4 low and SALL4 high cells. As shown, each of Cmpd 2, Cmpd 6, and Cmpd 7 had excellent EC₅₀ and selectivity between SALL4 low and SALL4 high cells. Cmpd 61 (missing the acrylic linker), on the other hand, had low selectivity and potency. The SALL4 high/low phenotypic screen clearly indicates that Cmpd 2, Cmpd 6, and Cmpd 7 had low EC50 values selectively in SALL4 high SNU398 cells compared to the negative control Cmpd 61.

Table 1 below shows the structures of compounds Cmpd 1 to Cmpd 80 of the library, according to the following structures:

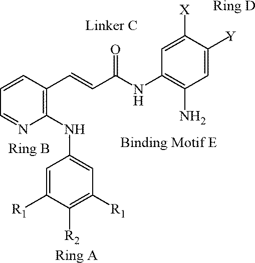

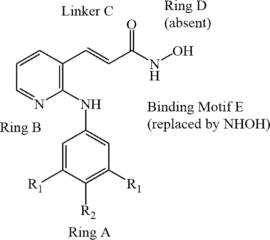

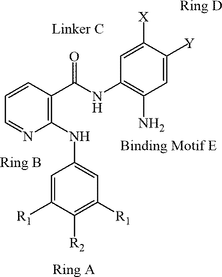

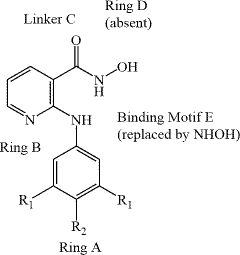

TABLE 1

Structures of compounds Cmpd 1 to Cmpd 80 of the Library

| | | | | | |
|---|---|---|---|---|---|
| Compound Code | Ring A | Ring B | Linker C | Ring D | Binding Motif E |
| Cmpd 1 | R1═R2═OMe | 2-Anilino | —CH═CH—CO— | X═Y═H | Phenylene diamine |
| Cmpd 2 | R1═R2═OMe | 2-Anilino | —CH═CH—CO— | X═OMe, Y═H | Phenylene diamine |
| Cmpd 3 | R1═R2═OMe | 2-Anilino | —CH═CH—CO— | X═Me, Y═H | Phenylene diamine |
| Cmpd 4 | R1═R2═OMe | 2-Anilino | —CH═CH—CO— | X═Y═Me | Phenylene diamine |
| Cmpd 5 | R1═R2═OMe | 2-Anilino | —CH═CH—CO— | X═F, Y═H | Phenylene diamine |
| Cmpd 6 | R1═R2═OMe | 2-Anilino | —CH═CH—CO— | X═H, Y═Cl | Phenylene diamine |
| Cmpd 7 | R1═R2═OMe | 2-Anilino | —CH═CH—CO— | X═Y═Cl | Phenylene diamine |
| Cmpd 8 | R1═R2═OMe | 2-Anilino | —CH═CH—CO— | NA | NHOH |
| Cmpd 9 | R1═H, R2═OMe | 2-Anilino | —CH═CH—CO— | X═Y═H | Phenylene diamine |
| Cmpd 10 | R1═H, R2═OMe | 2-Anilino | —CH═CH—CO— | X═OMe, Y═H | Phenylene diamine |
| Cmpd 11 | R1═H, R2═OMe | 2-Anilino | —CH═CH—CO— | X═Me, Y═H | Phenylene diamine |
| Cmpd 12 | R1═H, R2═OMe | 2-Anilino | —CH═CH—CO— | X═Y═Me | Phenylene diamine |
| Cmpd 13 | R1═H, R2═OMe | 2-Anilino | —CH═CH—CO— | X═F, Y═H | Phenylene diamine |
| Cmpd 14 | R1═H, R2═OMe | 2-Anilino | —CH═CH—CO— | X═H, Y═Cl | Phenylene diamine |
| Cmpd 15 | R1═H, R2═OMe | 2-Anilino | —CH═CH—CO— | X═Y═Cl | Phenylene diamine |
| Cmpd 16 | R1═H, R2═OMe | 2-Anilino | —CH═CH—CO— | NA | NHOH |
| Cmpd 17 | R1═H, R2═F | 2-Anilino | —CH═CH—CO— | X═Y═H | Phenylene diamine |
| Cmpd 18 | R1═H, R2═F | 2-Anilino | —CH═CH—CO— | X═OMe, Y═H | Phenylene diamine |
| Cmpd 19 | R1═H, R2═F | 2-Anilino | —CH═CH—CO— | X═Me, Y═H | Phenylene diamine |
| Cmpd 20 | R1═H, R2═F | 2-Anilino | —CH═CH—CO— | X═Y═Me | Phenylene diamine |
| Cmpd 21 | R1═H, R2═F | 2-Anilino | —CH═CH—CO— | X═F, Y═H | Phenylene diamine |
| Cmpd 22 | R1═H, R2═F | 2-Anilino | —CH═CH—CO— | X═H, Y═Cl | Phenylene diamine |
| Cmpd 23 | R1═H,R2═F | 2-Anilino | —CH═CH—CO— | X═Y═Cl | Phenylene diamine |
| Cmpd 24 | R1═H, R2═F | 2-Anilino | —CH═CH—CO— | NA | NHOH |
| Cmpd 25 | R1═H, R2═CF3 | 2-Anilino | —CH═CH—CO— | X═Y═H | Phenylene diamine |
| Cmpd 26 | R1═H, R2═CF3 | 2-Anilino | —CH═CH—CO— | X═OMe, Y═H | Phenylene diamine |
| Cmpd 27 | R1═H, R2═CF3 | 2-Anilino | —CH═CH—CO— | X═Me, Y═H | Phenylene diamine |
| Cmpd 28 | R1═H, R2═CF3 | 2-Anilino | —CH═CH—CO— | X═Y═Me | Phenylene diamine |
| Cmpd 29 | R1═H, R2═CF3 | 2-Anilino | —CH═CH—CO— | X═F, Y═H | Phenylene diamine |
| Cmpd 30 | R1═H,R2═CF3 | 2-Anilino | —CH═CH—CO— | X═H, Y═Cl | Phenylene diamine |
| Cmpd 31 | R1═H, R2═CF3 | 2-Anilino | —CH═CH—CO— | X═Y═Cl | Phenylene diamine |
| Cmpd 32 | R1═H, R2═CF3 | 2-Anilino | —CH═CH—CO— | NA | NHOH |
| Cmpd 33 | R1═H, R2═Cl | 2-Anilino | —CH═CH—CO— | X═Y═H | Phenylene diamine |
| Cmpd 34 | R1═H, R2═Cl | 2-Anilino | —CH═CH—CO— | X═OMe, Y═H | Phenylene diamine |
| Cmpd 35 | R1═H, R2═Cl | 2-Anilino | —CH═CH—CO— | X═Me, Y═H | Phenylene diamine |
| Cmpd 36 | R1═H, R2═Cl | 2-Anilino | —CH═CH—CO— | X═Y═Me | Phenylene diamine |
| Cmpd 37 | R1═H, R2═Cl | 2-Anilino | —CH═CH—CO— | X═F, Y═H | Phenylene diamine |
| Cmpd 38 | R1═H, R2═Cl | 2-Anilino | —CH═CH—CO— | X═H, Y═Cl | Phenylene diamine |
| Cmpd 39 | R1═H, R2═Cl | 2-Anilino | —CH═CH—CO— | X═Y═Cl | Phenylene diamine |
| Cmpd 40 | R1═H, R2═Cl | 2-Anilino | —CH═CH—CO— | NA | NHOH |
| Cmpd 41 | R1═R2═OMe | 2-Anilino | Carbonyl(-CO-) | X═Y═H | Phenylene diamine |
| Cmpd 42 | R1═R2═OMe | 2-Anilino | Carbonyl(-CO-) | X═OMe, Y═H | Phenylene diamine |
| Cmpd 43 | R1═R2═OMe | 2-Anilino | Carbonyl(-CO-) | X═Me, Y═H | Phenylene diamine |
| Cmpd 44 | R1═R2═OMe | 2-Anilino | Carbonyl(-CO-) | X═Y═Me | Phenylene diamine |
| Cmpd 45 | R1═R2═OMe | 2-Anilino | Carbonyl(-CO-) | X═F, Y═H | Phenylene diamine |
| Cmpd 46 | R1═R2═OMe | 2-Anilino | Carbonyl(-CO-) | X═Cl, Y═H | Phenylene diamine |
| Cmpd 47 | R1═R2═OMe | 2-Anilino | Carbonyl(-CO-) | X═Y═Cl | Phenylene diamine |
| Cmpd 48 | R1═R2═OMe | 2-Anilino | Carbonyl(-CO-) | NA | NHOH |
| Cmpd 49 | R1═H, R2═OMe | 2-Anilino | Carbonyl(-CO-) | X═Y═H | Phenylene diamine |
| Cmpd 50 | R1═H, R2═OMe | 2-Anilino | Carbonyl(-CO-) | X═OMe, Y═H | Phenylene diamine |
| Cmpd 51 | R1═H, R2═OMe | 2-Anilino | Carbonyl(-CO-) | X═Me, Y═H | Phenylene diamine |
| Cmpd 52 | R1═H, R2═OMe | 2-Anilino | Carbonyl(-CO-) | X═Y═Me | Phenylene diamine |
| Cmpd 53 | R1═H, R2═OMe | 2-Anilino | Carbonyl(-CO-) | X═F, Y═H | Phenylene diamine |
| Cmpd 54 | R1═H, R2═OMe | 2-Anilino | Carbonyl(-CO-) | X═Cl, Y═H | Phenylene diamine |
| Cmpd 55 | R1═H, R2═OMe | 2-Anilino | Carbonyl(-CO-) | X═Y═Cl | Phenylene diamine |
| Cmpd 56 | R1═H, R2═OMe | 2-Anilino | Carbonyl(-CO-) | NA | NHOH |
| Cmpd 57 | R1═H, R2═F | 2-Anilino | Carbonyl(-CO-) | X═Y═H | Phenylene diamine |
| Cmpd 58 | R1═H, R2═F | 2-Anilino | Carbonyl(-CO-) | X═OMe, Y═H | Phenylene diamine |
| Cmpd 59 | R1═H,R2═F | 2-Anilino | Carbonyl(-CO-) | X═Me, Y═H | Phenylene diamine |
| Cmpd 60 | R1═H, R2═F | 2-Anilino | Carbonyl(-CO-) | X═Y═Me | Phenylene diamine |
| Cmpd 61 | R1═H, R2═F | 2-Anilino | Carbonyl(-CO-) | X═F, Y═H | Phenylene diamine |
| Cmpd 62 | R1═H, R2═F | 2-Anilino | Carbonyl(-CO-) | X═Cl, Y═H | Phenylene diamine |
| Cmpd 63 | R1═H, R2═F | 2-Anilino | Carbonyl(-CO-) | X═Y═Cl | Phenylene diamine |
| Cmpd 64 | R1═H, R2═F | 2-Anilino | Carbonyl(-CO-) | NA | NHOH |
| Cmpd 65 | R1═H, R2═CF3 | 2-Anilino | Carbonyl(-CO-) | X═Y═H | Phenylene diamine |
| Cmpd 66 | R1═H,R2═CF3 | 2-Anilino | Carbonyl(-CO-) | X═OMe, Y═H | Phenylene diamine |
| Cmpd 67 | R1═H, R2═CF3 | 2-Anilino | Carbonyl(-CO-) | X═Me, Y═H | Phenylene diamine |
| Cmpd 68 | R1═H, R2═CF3 | 2-Anilino | Carbonyl(-CO-) | X═Y═Me | Phenylene diamine |
| Cmpd 69 | R1═H, R2═CF3 | 2-Anilino | Carbonyl(-CO-) | X═F, Y═H | Phenylene diamine |
| Cmpd 70 | R1═H, R2═CF3 | 2-Anilino | Carbonyl(-CO-) | X═Cl, Y═H | Phenylene diamine |
| Cmpd 71 | R1═H, R2═CF3 | 2-Anilino | Carbonyl(-CO-) | X═Y═Cl | Phenylene diamine |
| Cmpd 72 | R1═H, R2═CF3 | 2-Anilino | Carbonyl(-CO-) | NA | NHOH |
| Cmpd 73 | R1═H, R2═Cl | 2-Anilino | Carbonyl(-CO-) | X═Y═H | Phenylene diamine |
| Cmpd 74 | R1═H, R2═Cl | 2-Anilino | Carbonyl(-CO-) | X═OMe, Y═H | Phenylene diamine |
| Cmpd 75 | R1═H, R2═Cl | 2-Anilino | Carbonyl(-CO-) | X═Me, Y═H | Phenylene diamine |
| Cmpd 76 | R1═H, R2═Cl | 2-Anilino | Carbonyl(-CO-) | X═Y═Me | Phenylene diamine |
| Cmpd 77 | R1═H,R2═Cl | 2-Anilino | Carbonyl(-CO-) | X═F, Y═H | Phenylene diamine |
| Cmpd 78 | R1═H, R2═Cl | 2-Anilino | Carbonyl(-CO-) | X═Cl, Y═H | Phenylene diamine |
| Cmpd 79 | R1═H, R2═Cl | 2-Anilino | Carbonyl(-CO-) | X═Y═Cl | Phenylene diamine |
| Cmpd 80 | R1═H, R2═Cl | 2-Anilino | Carbonyl(-CO-) | NA | NHOH |

Compounds of the library were subjected to a variety of tests, including an Alpha Assay (RBBP4 binding); FP assay (RBBP4 binding); Cell-based 1299 (low SALL4) assay; Cell-based 549 (low SALL4) assay; Cell-based 661 (high SALL4) assay; Cell-based SNU-387 (SALL4 low) assay; Cell-based SNU-398 (SALL4 high) assay; Cell-based MDA-MB231 assay; Pan-HDAC binding assay; Tubulin Polymerisation Inhibition assay; SALL4 protein expression down regulation assay; SALL4 RNA expression down regulation assay; Other RNA expression changes assay; Permeability assay (PAMPA); Microsomal stability assay; and in vivo PK assay.

ALPHA assay measures binding between SALL4 and RBBp4, an inhibitor could block the binding and reflected in low reading of the ALPHA assay. FP assay also measures binding between SALL4 and RBBp4, an inhibitor results in higher reading. Cell-based assays are measuring cell viability with different concentration of compounds. Cell based 1299 refers to cell viability assay using H1299 cells; Cell-based SNU387 refers to cell viability assay using SNU387 cells; Cell based assay-SNU-398 refers to cell viability assay using SNU398 cells; Cell based MDA-MB231 refers to cell viability assay using MDA-MB231 cells.

Pan-HDAC binding assay measures HDAC inhibitory activity of a compound. Tubulin Polymerisation inhibition assays measure inhibition activity of a compound towards tubulin polymerization. SALL4 protein expression down regulation assay changed to western blot analysis, SALL4 RNA expression down regulation assay changed to real-time PCR assay. Permeability assay measures the (PAMPA) measures permeability of a compound across an artificial membrane. The microsomal stability assay measures rate of disappearance of a test compound over time in liver microsomes. In vivo PK assay measures biodistribution of the test compound in mice.

Table 2 below shows assay results.

Table 2: Assay results for the library, showing Alpha Assay (RBBP4 binding); FP assay (RBBP4 binding); Cell-based 1299 (low SALL4) assay; Cell-based 549 (low SALL4) assay; Cell-based 661 (high SALL4) assay; Cell-based SNU-387 (SALL4 low) assay; Cell-based SNU-398 (SALL4 high) assay; Cell-based MDA-MB231 assay; Pan-HDAC binding assay; Tubulin Polymerisation Inhibition assay; SALL4 protein expression down regulation assay; SALL4 RNA expression down regulation assay; Other RNA expression changes assay: Permeability assay (PAMPA); Microsomal stability assay; and in vivo PK assay results.

[symbol]

FIG. 3 shows Western blots showing SALL4 protein down regulation upon treatment of N-(2-aminophenyl)-prop-2-enamide derivatives. In particular, FIG. 3 shows results of testing with Cmpd 2, Cmpd 6, Cmpd 7, and Cmpd 61 for SALL4-related effects, also compared with entinostat and JQ1.

As shown by these results, certain compounds from the library may be relatively poor HDAC binders, however may still provide an important inhibitory effect showing selectivity in phenotypic screening for SALL4-high cells (both lung and liver cancer). Results further indicate that treatment may result in a down-regulation of SALL4 protein expression (as measured by Western blot). Cells were treated with compounds for 24 hours and harvested. The cell lysates were prepared and Western blotting was performed. When compared to DMSO treated cells, the cells treated with compounds have significantly lower amount of detectable SALL4 bands on the Western blot.

The SNU398 cells were incubated with the compounds (Cmpd1 to Cmpd80) for indicated time in the figure. Cells were then harvested by cell scraper and washed with PBS. The collected pellets were lysed with RIPA buffer (50 mM Tris, 150 mM NaC1, 1% TritonX-100, 0.5% sodium deoxycholate, and 0.1% SDS) supplemented with protease inhibitor cocktail. The extracted protein lysates were denatured with 4X SDS sample buffer (200mM Tris-HCl pH 6.8, 8% SDS, 40% glycerol, 4% β-mercaptoethanol, 50mM EDTA, 0.08% bromophenol blue) at 99° C. for 5 minutes. Equal amount of protein were subjected to electrophoresis in 8% SDS-PAGE gel, and then transferred to PVDF membrane. After blocking in Blocking One (Nacalai Tesque), the membrane was probed with primary antibodies to SALL4 (Santa Cruz Biotechnology, sc-101147) and β-Actin (Santa Cruz Biotechnology, sc-47778) overnight at 4° C. After washing with TBS-T, membrane was incubated with secondary HRP-conjugated antibody to mouse for 1 hour (Santa Cruz Biotechnology, sc-2005). Luminata™ western HRP substrate (Millipore) was applied to the membrane for visualization.

FIG. 4 shows results for in vivo transgenic mice experiments showing SALL4 high tumor responds to N-(2-aminophenyl)-prop-2-enamide derivatives. In particular, FIG. 4 shows results for in vivo xenotransplant testing for the Cmpd 6 compound. Mice treated with Compound 6 had significant (P<0.05) smaller xenografts both in size and weight.

FIG. 5 shows a diagram of a proposed mechanism for SALL4 inhibition and restoration of PTEN (or other tumor suppressor gene) in a tumor cell by an N-(2-aminophenyl)-prop-2-enamide compound as described herein. Without wishing to be bound by theory, treatment with the N-(2-aminophenyl)-prop-2-enamide compound may inhibit SALL4 and/or prevent formation of (or dissociate) SALL4-NuRD complex, which may result in expression of one or more tumor suppressor genes such as PTEN. In certain embodiments, and without wishing to be bound by theory, it is contemplated that SALL4 may bind to NuRD to repress the tumor suppressor genes, by co-occupying the promoters and may suppress transcriptions. It is contemplated that in certain embodiments, and without wishing to be bound by theory, once SALL4 is degraded or decreased, the formation of the complex may be affected, hence releasing the transcription.

FIG. 6 shows results in which Compound 6 (Cmpd 6) and other derivatives (as indicated) were tested in two lung cancer cell lines, where A549 is SALL4-low, and H661 is SALL4-high. Cells were treated with compound 6 at 1 µM and 2.5 µM. Amounts for other cmpds are shown. Cells were harvested at 48 hrs and the cell lysates were subjected to western blot analysis and Q-PCR analysis for SALL4 protein and RNA level respectively. As shown in the upper panel of FIG. 6, in the western blot analysis, it was found that Compound 6 could reduce SALL4 protein level at 2.5 µM. As shown in the lower panel of FIG. 6, using Q-PCR, it was shown that SALL4 RNA level was reduced by Compound 6 in H661 cells.

FIG. 7 shows results indicating that Compound 6 (Cmpd 6) binds to SALL4. In the upper panel of FIG. 7, a fluorescence-based binding assay named Thermal Shift Assay was used to assess the binding of compound 6 to SALL4 1-300 protein based on changes in unfolding transition. The result shows that incubation of SALL4 1-300 with 100 mM compound 6 resulted in a melting shift of 4.7° C., indicating compound 6 binds to SALL4. In the lower panel of FIG. 7, compound 6 binding to SALL4 was accessed by 1H NMR Experiments and Saturation Transfer Difference (STD) using ¹⁵N SALL4 1-300 and Compound 6. The samples were screened using Bruker BioSpin AVANCE II 600 MHz spectrometer equipped with a CPPTCI{ 19F} cryoprobe and SampleJet auto-sampler. Compound 6 weak binding event to SALL4 1-300 was identified based on the increase in transverse relaxation rate (R2) observed in the presence of SALL4 1-300 protein.

FIG. 8 shows results of a pharmacokinetic study of compound 6. To understand the *bioavailability of compound 6 (Cmpd 6), compound 6 was given orally (15 mg/kg), or via intravenous injection (5 mg/kg) to Swiss albino mice. Compound 6 was dissolved in water containing 3% DMSO and 10% hydroxyl propyl-b-cyclodextrin (EncapsinTM) for the i.v. dose, and in water containing 5% DMSO and 9.5% Encapsin for the oral dose. Blood samples were collected at 0.083, 0.25, 0.5, 1, 2, 4, 6, 8 and 24 hrs. The whole blood concentration was measured by LC-MS/MS. From the oral route, 90% of compound 6 was cleared at 2 hrs. For the intravenous route, 90% of compound 6 was cleared at 4 hrs.

FIG. 9 shows results in which Compound 6 (Cmpd 6) was tested for metabolic stability using liver microsomes. 1 µM of compound 6 was incubated with 3.33 mg/ml liver microsomes and 2.5 mM NADPH for 0, 5, 10, 30 and 60 minutes. The mixture were subjected to LC-MS/MS to measure remaining compound after the incubation. The result shows that compound 6 has medium permeability status at 45.88%QH.

FIG. 10 shows results in which PAMPA permeability assay was performed on compound 6 and some comparators. 50 µM of Compound 6 prepared in pION buffer was added to bottom of UV plate. GIT-0 solution was added and the solution was incubated for 4 hours. The plate spectrum was read using spectrophotometer in scanning mode from 200 nm to 500 nm using PAMPA pION software. Result shows that compound 6 has high permeability in PAMPA assay.

### Example 3 - Additional Compounds

This Example sets out further compounds that are contemplated herein, based on the results described in Examples 1 and 2 above. It is contemplated that in certain embodiments one or more of the compounds described in this Example (see below) may be for use in treatment of high SALL4 cancers, for example. Alternatively, or in addition, it is contemplated that in certain embodiments one or more of the compounds described in this Example (see below) may be for use as a comparator, for use as structural probes to investigate pharmacophore features and/or interaction(s) with protein or enzyme target(s) (such as SALL4, or SALL4-RBBP4 interaction), and/or as binders or inhibitors of SALL4 and/or SALL4-RBBP4, or any combinations thereof.

In certain embodiments, compounds encompassed by the following formula:

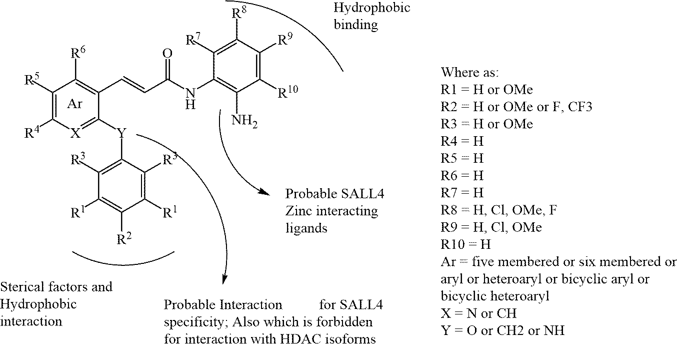

are provided herein.

In certain embodiments, compounds with the following modifications at Ring A are provided herein:

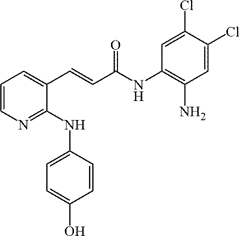 [SMILES: Nc1cc(Cl)c(Cl)cc1NC(=O)/C=C/c1cccnc1Nc1ccc(O)cc1]

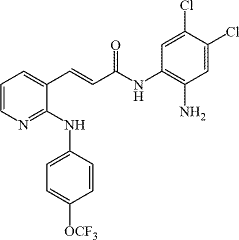 [SMILES: Nc1cc(Cl)c(Cl)cc1NC(=O)/C=C/c1cccnc1Nc1ccc(OC(F)(F)F)cc1]

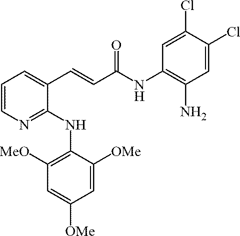 [SMILES: COc1cc(OC)c(Nc2ncccc2/C=C/C(=O)Nc2cc(Cl)c(Cl)cc2N)c(OC)c1]

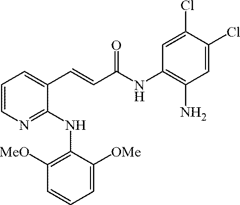 [SMILES: COc1cccc(OC)c1Nc1ncccc1/C=C/C(=O)Nc1cc(Cl)c(Cl)cc1N]

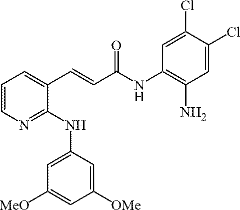 [SMILES: COc1cc(Nc2ncccc2/C=C/C(=O)Nc2cc(Cl)c(Cl)cc2N)cc(OC)c1]

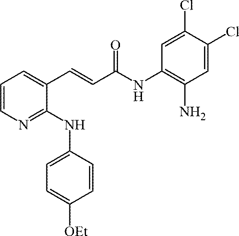 [SMILES: CCOc1ccc(Nc2ncccc2/C=C/C(=O)Nc2cc(Cl)c(Cl)cc2N)cc1]

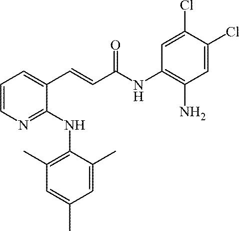 [SMILES: Cc1cc(C)c(Nc2ncccc2/C=C/C(=O)Nc2cc(Cl)c(Cl)cc2N)c(C)c1]

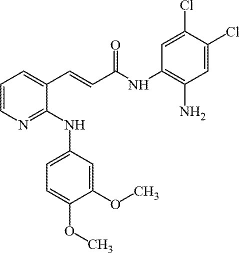 [SMILES: COc1ccc(Nc2ncccc2/C=C/C(=O)Nc2cc(Cl)c(Cl)cc2N)cc1OC]

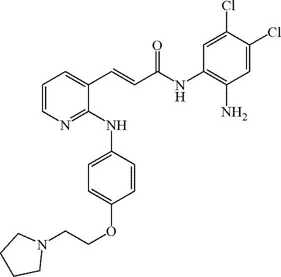 [SMILES: Nc1cc(Cl)c(Cl)cc1NC(=O)/C=C/c1cccnc1Nc1ccc(OCCN2CCCC2)cc1]

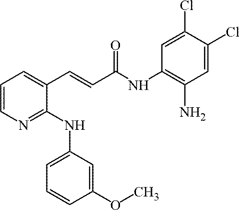 [SMILES: COc1cccc(Nc2ncccc2/C=C/C(=O)Nc2cc(Cl)c(Cl)cc2N)c1]

In certain embodiments, compounds with the following modifications at Ring A/Ring B are provided herein:

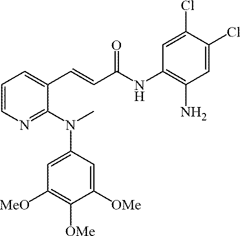 [SMILES: COc1cc(N(C)c2ncccc2/C=C/C(=O)Nc2cc(Cl)c(Cl)cc2N)cc(OC)c1OC]

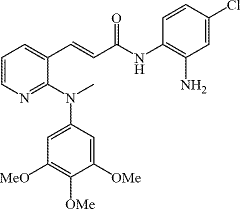 [SMILES: COc1cc(N(C)c2ncccc2/C=C/C(=O)Nc2ccc(Cl)cc2N)cc(OC)c1OC]

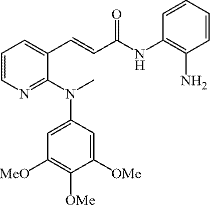 [SMILES: COc1cc(N(C)c2ncccc2/C=C/C(=O)Nc2ccccc2N)cc(OC)c1OC]

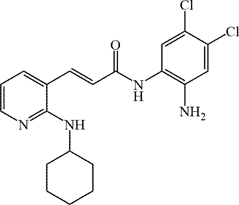 [SMILES: Nc1cc(Cl)c(Cl)cc1NC(=O)/C=C/c1cccnc1NC1CCCCC1]

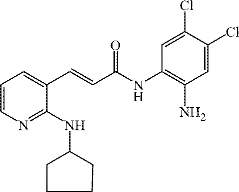 [SMILES: Nc1cc(Cl)c(Cl)cc1NC(=O)/C=C/c1cccnc1NC1CCCC1]

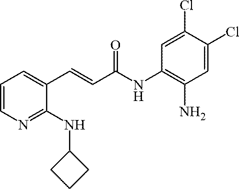 [SMILES: Nc1cc(Cl)c(Cl)cc1NC(=O)/C=C/c1cccnc1NC1CCC1]

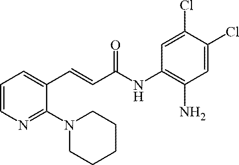 [SMILES: Nc1cc(Cl)c(Cl)cc1NC(=O)/C=C/c1cccnc1N1CCCCC1]

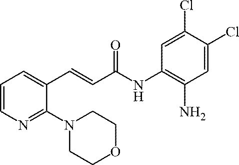 [SMILES: Nc1cc(Cl)c(Cl)cc1NC(=O)/C=C/c1cccnc1N1CCOCC1]

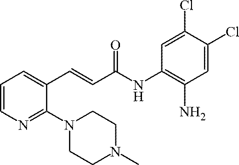 [SMILES: CN1CCN(c2ncccc2/C=C/C(=O)Nc2cc(Cl)c(Cl)cc2N)CC1]

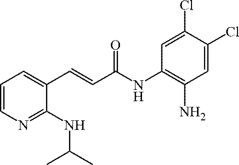 [SMILES: CC(C)Nc1ncccc1/C=C/C(=O)Nc1cc(Cl)c(Cl)cc1N]

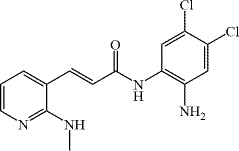 [SMILES: CNc1ncccc1/C=C/C(=O)Nc1cc(Cl)c(Cl)cc1N]

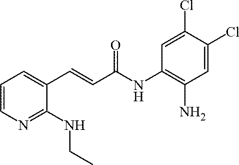 [SMILES: CCNc1ncccc1/C=C/C(=O)Nc1cc(Cl)c(Cl)cc1N]

In certain embodiments, compounds with the following modifications at pyridyl Ring B are provided herein:

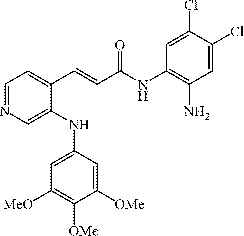 [SMILES: COc1cc(Nc2cnccc2/C=C/C(=O)Nc2cc(Cl)c(Cl)cc2N)cc(OC)c1OC]

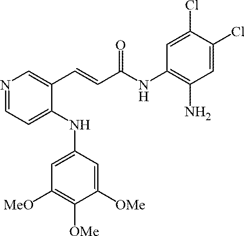 [SMILES: COc1cc(Nc2ccncc2/C=C/C(=O)Nc2cc(Cl)c(Cl)cc2N)cc(OC)c1OC]

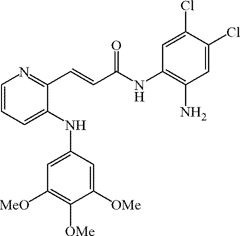 [SMILES: COc1cc(Nc2cccnc2/C=C/C(=O)Nc2cc(Cl)c(Cl)cc2N)cc(OC)c1OC]

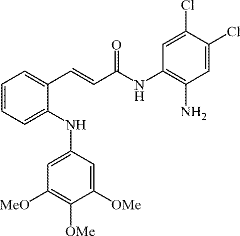 [SMILES: COc1cc(Nc2ccccc2/C=C/C(=O)Nc2cc(Cl)c(Cl)cc2N)cc(OC)c1OC]

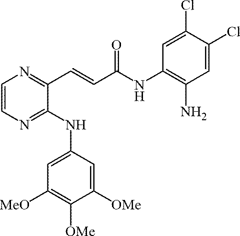 [SMILES: COc1cc(Nc2nccnc2/C=C/C(=O)Nc2cc(Cl)c(Cl)cc2N)cc(OC)c1OC]

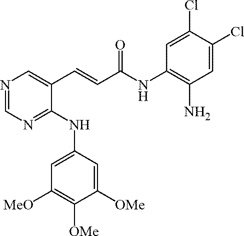 [SMILES: COc1cc(Nc2ncncc2/C=C/C(=O)Nc2cc(Cl)c(Cl)cc2N)cc(OC)c1OC]

In certain embodiments, compounds with the following modifications at Linker C are provided herein:

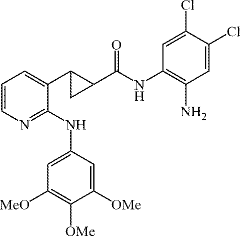 [SMILES: COc1cc(Nc2ncccc2C2CC2C(=O)Nc2cc(Cl)c(Cl)cc2N)cc(OC)c1OC]

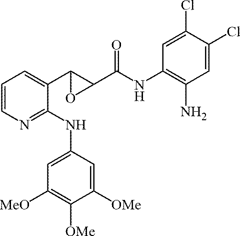 [SMILES: COc1cc(Nc2ncccc2C2OC2C(=O)Nc2cc(Cl)c(Cl)cc2N)cc(OC)c1OC]

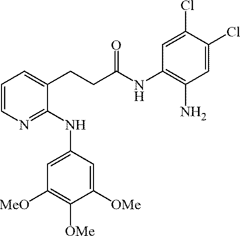 [SMILES: COc1cc(Nc2ncccc2CCC(=O)Nc2cc(Cl)c(Cl)cc2N)cc(OC)c1OC]

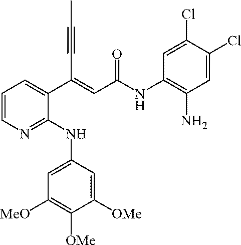 [SMILES: CC#C/C(=C\C(=O)Nc1cc(Cl)c(Cl)cc1N)c1cccnc1Nc1cc(OC)c(OC)c(OC)c1]

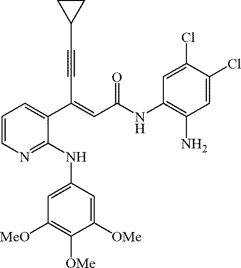 [SMILES: COc1cc(Nc2ncccc2/C(C#CC2CC2)=C/C(=O)Nc2cc(Cl)c(Cl)cc2N)cc(OC)c1OC]

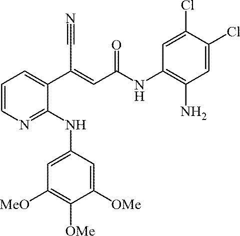 [SMILES: COc1cc(Nc2ncccc2/C(C#N)=C/C(=O)Nc2cc(Cl)c(Cl)cc2N)cc(OC)c1OC]

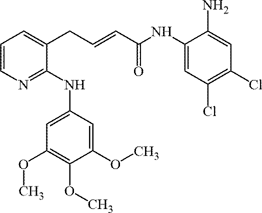 [SMILES: COc1cc(Nc2ncccc2C/C=C/C(=O)Nc2cc(Cl)c(Cl)cc2N)cc(OC)c1OC]

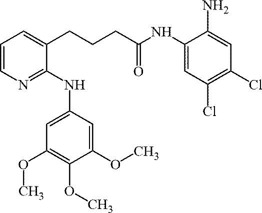 [SMILES: COc1cc(Nc2ncccc2CCCC(=O)Nc2cc(Cl)c(Cl)cc2N)cc(OC)c1OC]

In certain embodiments, compounds with the following modifications at Amide Ring D are provided herein:

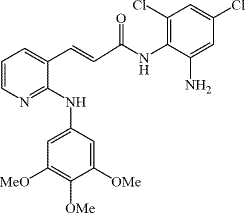 [SMILES: COc1cc(Nc2ncccc2/C=C/C(=O)Nc2c(N)cc(Cl)cc2Cl)cc(OC)c1OC]

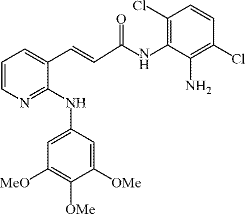 [SMILES: COc1cc(Nc2ncccc2/C=C/C(=O)Nc2c(Cl)ccc(Cl)c2N)cc(OC)c1OC]

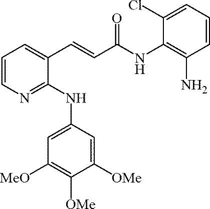 [SMILES: COc1cc(Nc2ncccc2/C=C/C(=O)Nc2c(N)cccc2Cl)cc(OC)c1OC]

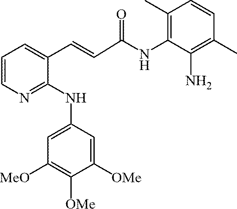 [SMILES: COc1cc(Nc2ncccc2/C=C/C(=O)Nc2c(C)ccc(C)c2N)cc(OC)c1OC]

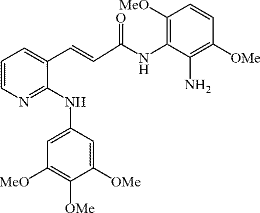 [SMILES: COc1ccc(OC)c(NC(=O)/C=C/c2cccnc2Nc2cc(OC)c(OC)c(OC)c2)c1N]

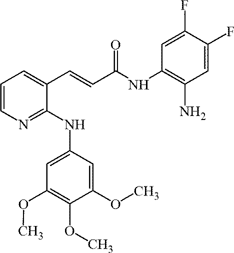 [SMILES: COc1cc(Nc2ncccc2/C=C/C(=O)Nc2cc(F)c(F)cc2N)cc(OC)c1OC]

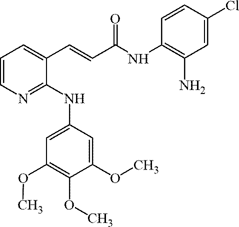 [SMILES: COc1cc(Nc2ncccc2/C=C/C(=O)Nc2ccc(Cl)cc2N)cc(OC)c1OC]

In certain embodiments, compounds with the following modifications at Binding Motif E are provided herein:

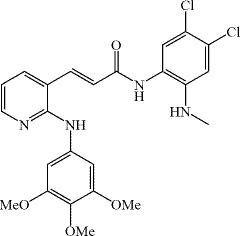 [SMILES: CNc1cc(Cl)c(Cl)cc1NC(=O)/C=C/c1cccnc1Nc1cc(OC)c(OC)c(OC)c1]

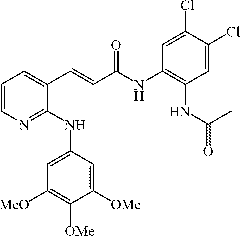 [SMILES: COc1cc(Nc2ncccc2/C=C/C(=O)Nc2cc(Cl)c(Cl)cc2NC(C)=O)cc(OC)c1OC]

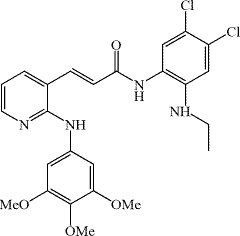 [SMILES: CCNc1cc(Cl)c(Cl)cc1NC(=O)/C=C/c1cccnc1Nc1cc(OC)c(OC)c(OC)c1]

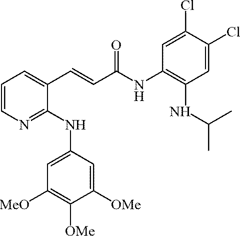 [SMILES: COc1cc(Nc2ncccc2/C=C/C(=O)Nc2cc(Cl)c(Cl)cc2NC(C)C)cc(OC)c1OC]

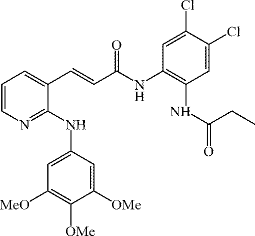 [SMILES: CCC(=O)Nc1cc(Cl)c(Cl)cc1NC(=O)/C=C/c1cccnc1Nc1cc(OC)c(OC)c(OC)c1]

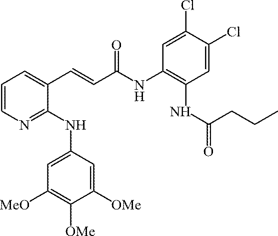 [SMILES: CCCC(=O)Nc1cc(Cl)c(Cl)cc1NC(=O)/C=C/c1cccnc1Nc1cc(OC)c(OC)c(OC)c1]

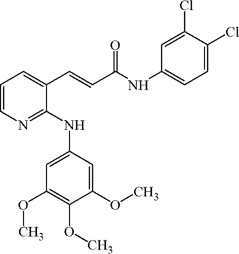 [SMILES: COc1cc(Nc2ncccc2/C=C/C(=O)Nc2ccc(Cl)c(Cl)c2)cc(OC)c1OC]

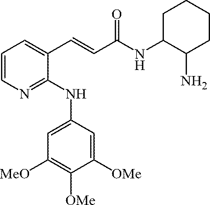 [SMILES: COc1cc(Nc2ncccc2/C=C/C(=O)NC2CCCCC2N)cc(OC)c1OC]

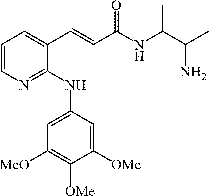 [SMILES: COc1cc(Nc2ncccc2/C=C/C(=O)NC(C)C(C)N)cc(OC)c1OC]

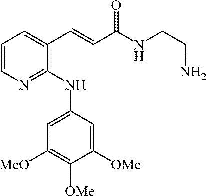 [SMILES: COc1cc(Nc2ncccc2/C=C/C(=O)NCCN)cc(OC)c1OC]

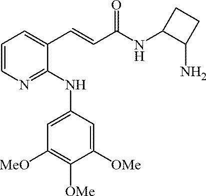 [SMILES: COc1cc(Nc2ncccc2/C=C/C(=O)NC2CCC2N)cc(OC)c1OC]

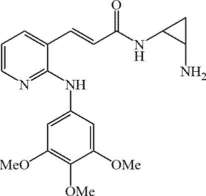 [SMILES: COc1cc(Nc2ncccc2/C=C/C(=O)NC2CC2N)cc(OC)c1OC]

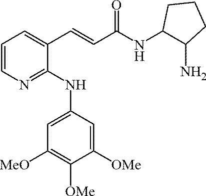 [SMILES: COc1cc(Nc2ncccc2/C=C/C(=O)NC2CCCC2N)cc(OC)c1OC]

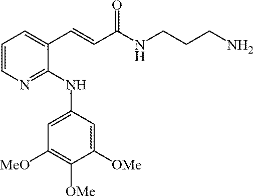 [SMILES: COc1cc(Nc2ncccc2/C=C/C(=O)NCCCN)cc(OC)c1OC]

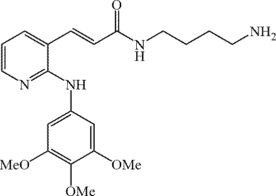 [SMILES: COc1cc(Nc2ncccc2/C=C/C(=O)NCCCCN)cc(OC)c1OC]

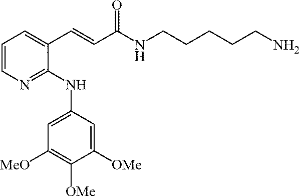 [SMILES: COc1cc(Nc2ncccc2/C=C/C(=O)NCCCCCN)cc(OC)c1OC]

In certain embodiments, compounds with the following modifications at Ring A (removed or altered) and Ring D (simplified) are provided herein:

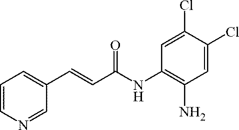 [SMILES: Nc1cc(Cl)c(Cl)cc1NC(=O)/C=C/c1cccnc1]

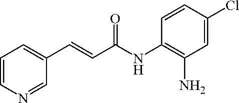 [SMILES: Nc1cc(Cl)ccc1NC(=O)/C=C/c1cccnc1]

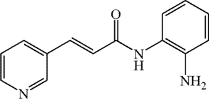 [SMILES: Nc1ccccc1NC(=O)/C=C/c1cccnc1]

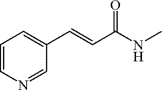

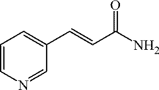 [SMILES: NC(=O)/C=C/c1cccnc1]

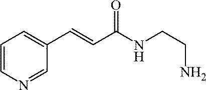 [SMILES: NCCNC(=O)/C=C/c1cccnc1]

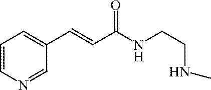

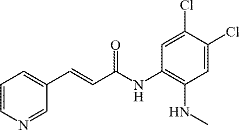 [SMILES: CNc1cc(Cl)c(Cl)cc1NC(=O)/C=C/c1cccnc1]

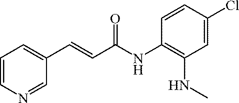 [SMILES: CNc1cc(Cl)ccc1NC(=O)/C=C/c1cccnc1]

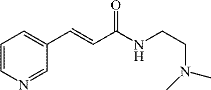 [SMILES: CN(C)CCNC(=O)/C=C/c1cccnc1]

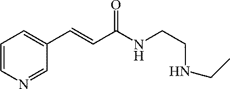 [SMILES: CCNCCNC(=O)/C=C/c1cccnc1]

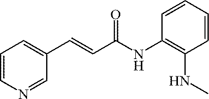 [SMILES: CNc1ccccc1NC(=O)/C=C/c1cccnc1]

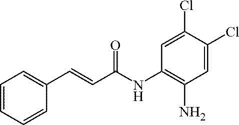 [SMILES: Nc1cc(Cl)c(Cl)cc1NC(=O)/C=C/c1ccccc1]

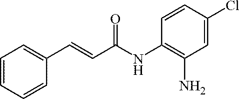 [SMILES: Nc1cc(Cl)ccc1NC(=O)/C=C/c1ccccc1]

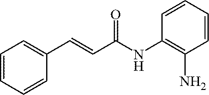 [SMILES: Nc1ccccc1NC(=O)/C=C/c1ccccc1]

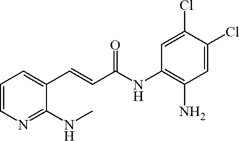 [SMILES: CNc1ncccc1/C=C/C(=O)Nc1cc(Cl)c(Cl)cc1N]

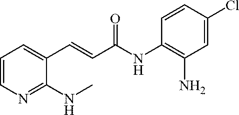 [SMILES: CNc1ncccc1/C=C/C(=O)Nc1ccc(Cl)cc1N]

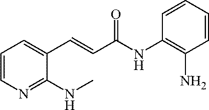 [SMILES: CNc1ncccc1/C=C/C(=O)Nc1ccccc1N]

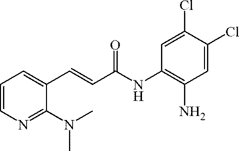 [SMILES: CN(C)c1ncccc1/C=C/C(=O)Nc1cc(Cl)c(Cl)cc1N]

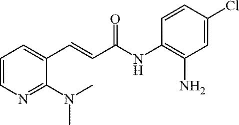 [SMILES: CN(C)c1ncccc1/C=C/C(=O)Nc1ccc(Cl)cc1N]

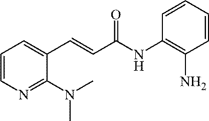 [SMILES: CN(C)c1ncccc1/C=C/C(=O)Nc1ccccc1N]

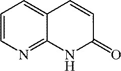 [SMILES: O=c1ccc2cccnc2[nH]1]

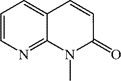

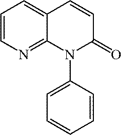 [SMILES: O=c1ccc2cccnc2n1-c1ccccc1]

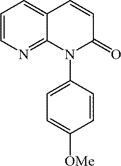 [SMILES: COc1ccc(-n2c(=O)ccc3cccnc32)cc1]

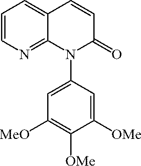 [SMILES: COc1cc(-n2c(=O)ccc3cccnc32)cc(OC)c1OC]

In certain embodiments, E3 ligase binder conjugated molcules are provided herein. Without wishing to be bound by theory, in certain embodiments it is contemplated that molecules as described herein may degrade SALL4 or its upstream targets by gluing the target protein with the E3 ligase. Accordingly, compounds acting as molecular glue/PROTAC/Degrader are contemplated herein, such as those which conjugate E3 ligase (CRBN/VHL/IAP/MDM2/XIAP) binders to the Prop-2-Enamide-type functionality. Accordingly, in embodiment, there is provided herein a compound comprising an E3 ligase binder conjugated or linked with a prop-2-enamide moiety or derivative as described herein. Representative examples of such molecules are provided below. In certain embodiments it is contemplated that Glutarimide, Lenalidomide, Pomalidomide and their isomers conjugated propenamides may induce CRBN (E3 ligase) binding mediated SALL4 (or its upstream target) ubiquitination/degradation. In certain embodiments it is contemplated that VHL binder conjugated propenamides may be designed to degrade SALL4 (or its upstream target) via VHL (E3 ligase) mediated degradation. Similarly, in certain embodiments it is contemplated that various other E3 ligase (MDM2/IAP/XIAP) mediated degradation of SALL4 (or its upstream target) may be be employed. Accordingly, in certain embodiments, E3 ligase binder conjugated molecules are provided herein, including (but not limited to) the following E3 ligase binder conjugated molecules (e.g. CRBN/VHL/IAP/MDM2/XIAP binders):

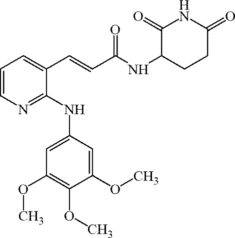 [SMILES: COc1cc(Nc2ncccc2/C=C/C(=O)NC2CCC(=O)NC2=O)cc(OC)c1OC]

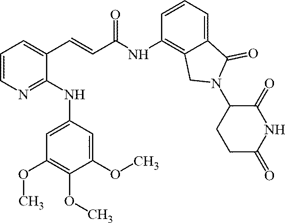 [SMILES: COc1cc(Nc2ncccc2/C=C/C(=O)Nc2cccc3c2CN(C2CCC(=O)NC2=O)C3=O)cc(OC)c1OC]

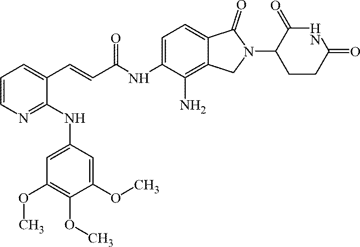 [SMILES: COc1cc(Nc2ncccc2/C=C/C(=O)Nc2ccc3c(c2N)CN(C2CCC(=O)NC2=O)C3=O)cc(OC)c1OC]

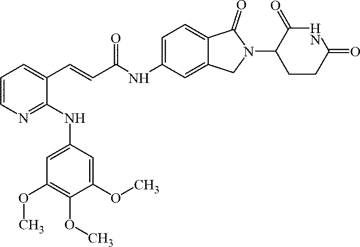 [SMILES: COc1cc(Nc2ncccc2/C=C/C(=O)Nc2ccc3c(c2)CN(C2CCC(=O)NC2=O)C3=O)cc(OC)c1OC]

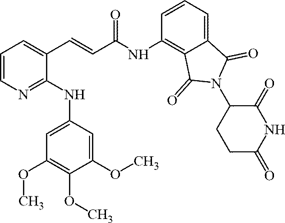 [SMILES: COc1cc(Nc2ncccc2/C=C/C(=O)Nc2cccc3c2C(=O)N(C2CCC(=O)NC2=O)C3=O)cc(OC)c1OC]

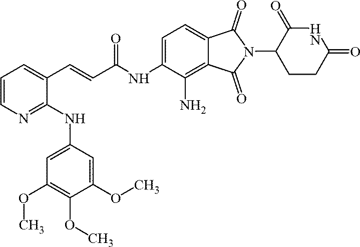 [SMILES: COc1cc(Nc2ncccc2/C=C/C(=O)Nc2ccc3c(c2N)C(=O)N(C2CCC(=O)NC2=O)C3=O)cc(OC)c1OC]

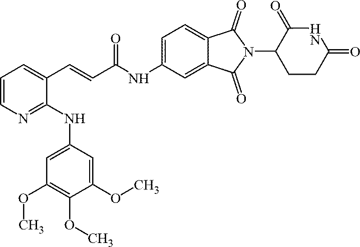 [SMILES: COc1cc(Nc2ncccc2/C=C/C(=O)Nc2ccc3c(c2)C(=O)N(C2CCC(=O)NC2=O)C3=O)cc(OC)c1OC]

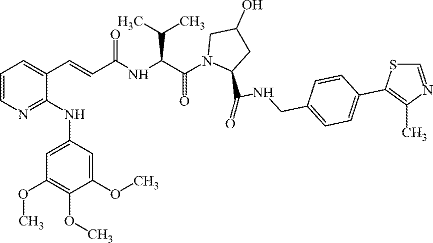 [SMILES: COc1cc(Nc2ncccc2/C=C/C(=O)N[C@H](C(=O)N2CC(O)C[C@H]2C(=O)NCc2ccc(-c3scnc3C)cc2)C(C)C)cc(OC)c1OC]

In certain embodiments, compounds having extended chain length and/or saturated bonds and/or extended double bonding are provided herein. PROTACs or zinc binders (like HDAC inhibitors) in general have lengthier linkers, and so compounds having increased linker length and/or saturated bonds and/or extended double bonding are also contemplated herein, including (but not limited to) the following compounds:

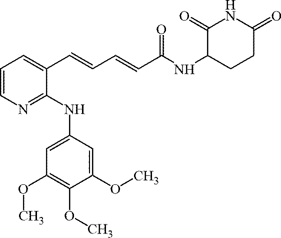 [SMILES: COc1cc(Nc2ncccc2/C=C/C=C/C(=O)NC2CCC(=O)NC2=O)cc(OC)c1OC]

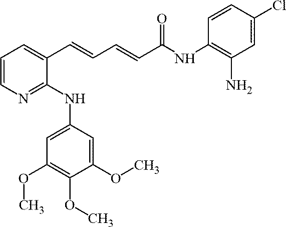 [SMILES: COc1cc(Nc2ncccc2/C=C/C=C/C(=O)Nc2ccc(Cl)cc2N)cc(OC)c1OC]

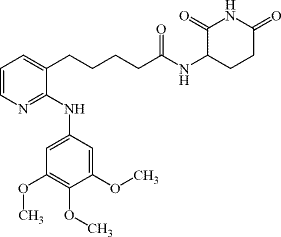 [SMILES: COc1cc(Nc2ncccc2CCCCC(=O)NC2CCC(=O)NC2=O)cc(OC)c1OC]

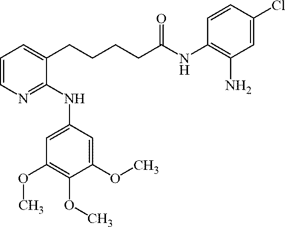 [SMILES: COc1cc(Nc2ncccc2CCCCC(=O)Nc2ccc(Cl)cc2N)cc(OC)c1OC]

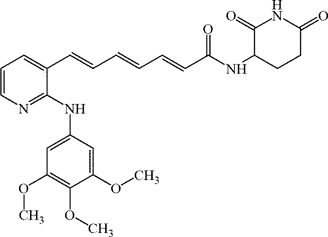 [SMILES: COc1cc(Nc2ncccc2/C=C/C=C/C=C/C(=O)NC2CCC(=O)NC2=O)cc(OC)c1OC]

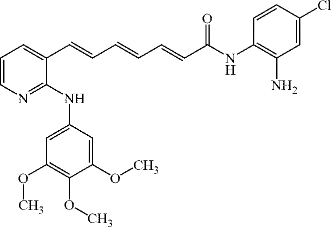 [SMILES: COc1cc(Nc2ncccc2/C=C/C=C/C=C/C(=O)Nc2ccc(Cl)cc2N)cc(OC)c1OC]

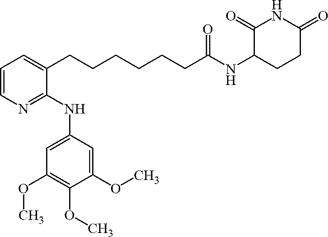 [SMILES: COc1cc(Nc2ncccc2CCCCCCC(=O)NC2CCC(=O)NC2=O)cc(OC)c1OC]

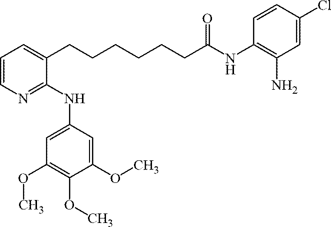 [SMILES: COc1cc(Nc2ncccc2CCCCCCC(=O)Nc2ccc(Cl)cc2N)cc(OC)c1OC]

### Listing of Embodiments

Embodiment 1: A compound of Formula I:

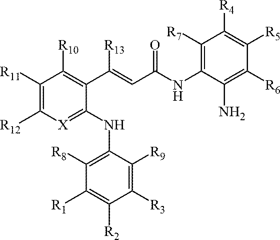

wherein
R₁ is H or -OCH₃;
R₂ is H, -OCH₃, -CF₃, or F;
R₃ is H or -OCH₃;
R₄ is H, C1, -OCH₃, -CH₃, F, or -NH₂;
R₅ is H, Cl, -OCH₃, -CH₃, F, or NH₂;
R₆ is H, C1, -OCH₃, or —CH₃;
R₇ is H, Cl, -OCH₃, or -CH₃;
R₈ is H, -OCH₃, or -CH₃;
R₉ is H, -OCH₃, or -CH₃;
each of R₁₀, R₁₁, and R₁₂ is H, or R₁₀ and R₁₁ together with the carbon atoms to which they are attached form a 5 or 6-membered aryl or heteroaryl ring and R₁₂ is H, or R₁₁ and R₁₂ together with the carbon atoms to which they are attached form a 5 or 6-membered aryl or heteroaryl ring and R₁₀ is H;
R₁₃ is H, -C≡C-CH₃, -C-C≡C₃H₅, or -C≡N; and
X is N or CH.

Embodiment 2: The compound of Embodiment 1, wherein R₁ is -OCH₃.

Embodiment 3: The compound of Embodiment 1 or 2, wherein R₂ is -OCH₃ or -CF₃.

Embodiment 4: The compound of any one of Embodiments 1-3, wherein R₃ is -OCH₃.

Embodiment 5: The compound of any one of Embodiments 1-4, wherein R₄ is H, Cl, -OCH₃, or -CH₃.

Embodiment 6: The compound of any one of Embodiments 1-5, wherein R₅ is H or C1.

Embodiment 7: The compound of any one of Embodiments 1-6, wherein R₆ is H.

Embodiment 8: The compound of any one of Embodiments 1-7, wherein R₇ is H.

Embodiment 9: The compound of any one of Embodiments 1-8, wherein R₈ is H.

Embodiment 10: The compound of any one of Embodiments 1-9, wherein R₉ is H.

Embodiment 11: The compound of any one of Embodiments 1-10, wherein R₁₀, R₁₁, and R₁₂ are each H.

Embodiment 12: The compound of any one of Embodiments 1-11, wherein R₁₃ is H.

Embodiment 13: The compound of any one of Embodiments 1-12, wherein X is N.

Embodiment 14: The compound of Embodiment 1, wherein the compound is:

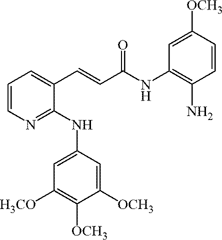 [SMILES: COc1ccc(N)c(NC(=O)/C=C/c2cccnc2Nc2cc(OC)c(OC)c(OC)c2)c1]

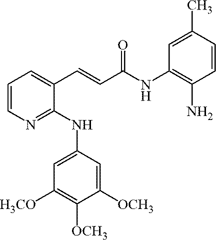 [SMILES: COc1cc(Nc2ncccc2/C=C/C(=O)Nc2cc(C)ccc2N)cc(OC)c1OC]

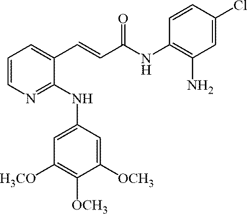 [SMILES: COc1cc(Nc2ncccc2/C=C/C(=O)Nc2ccc(Cl)cc2N)cc(OC)c1OC]

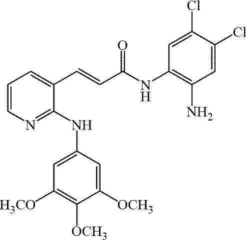 [SMILES: COc1cc(Nc2ncccc2/C=C/C(=O)Nc2cc(Cl)c(Cl)cc2N)cc(OC)c1OC]

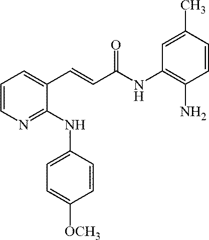 [SMILES: COc1ccc(Nc2ncccc2/C=C/C(=O)Nc2cc(C)ccc2N)cc1]

or

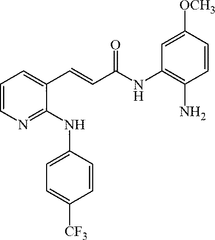 [SMILES: COc1ccc(N)c(NC(=O)/C=C/c2cccnc2Nc2ccc(C(F)(F)F)cc2)c1]

Embodiment 15: The compound of Embodiment 1 or Embodiment 14, wherein the compound is:

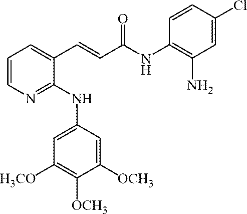 [SMILES: COc1cc(Nc2ncccc2/C=C/C(=O)Nc2ccc(Cl)cc2N)cc(OC)c1OC]

Embodiment 16: A composition comprising the compound of any one of Embodiments 1-15, and a pharmaceutically acceptable carrier, excipient, or diluent.

Embodiment 17: The compound of any one of Embodiments 1-15, or the composition of Embodiment 16, for use in the treatment of cancer in a cell or a subject in need thereof.

Embodiment 18: Use of the compound of any one of Embodiments 1-15, or the composition of Embodiment 16, for the treatment of cancer in a cell or a subject in need thereof

Embodiment 19: Use of the compound of any one of Embodiments 1-15, or the composition of Embodiment 16, in the manufacture of a medicament for use in the treatment of cancer in a cell or a subject in need thereof.

Embodiment 20: The compound, composition, or use according to any one of Embodiments 17-19, wherein the cancer is a SALL4-expressing cancer.

Embodiment 21: The compound, composition, or use according to Embodiment 20, wherein the cancer is a SALL4-expressing cancer having a high level of SALL4 expression.

Embodiment 22: The compound, composition, or use according to any one of Embodiments 17-21, wherein the cancer is lung cancer, liver cancer, or breast cancer.

Embodiment 23: The compound, composition, or use according to Embodiment 22, wherein the cancer is NSCLC cancer, cervical cancer, or germ cell cancer.

Embodiment 24: A method for treating cancer in a cell or subject in need thereof, said method comprising:

administering a compound of any one of Embodiments 1-15, or a composition of Embodiment 16, to the cell or subject.

Embodiment 25: The method of Embodiment 24, wherein the cancer is a SALL4-expressing cancer.

Embodiment 26: The method of Embodiment 25, wherein the cancer is a SALL4-expressing cancer having a high level of SALL4 expression.

Embodiment 27: The method according to any one of Embodiments 24-26, wherein the cancer is lung cancer, liver cancer, or breast cancer.

Embodiment 28: The method according to Embodiment 27, wherein the cancer is NSCLC cancer, cervical cancer, or germ cell cancer.

Embodiment 29: A method for preparing an N-(2-aminophenyl)-prop-2-enamide derivative, said method comprising:
reacting a compound of formula 1 with a compound of formula 2 to form a compound of formula 3:

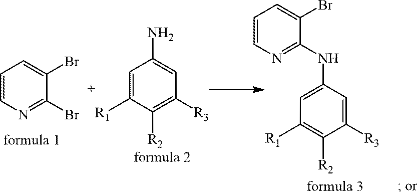

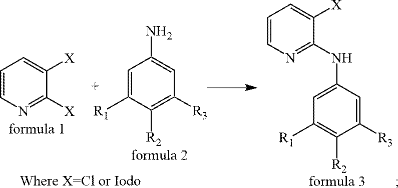

reacting the compound of formula 3 with a compound of formula 4, and deprotecting, to form a compound of formula 5:

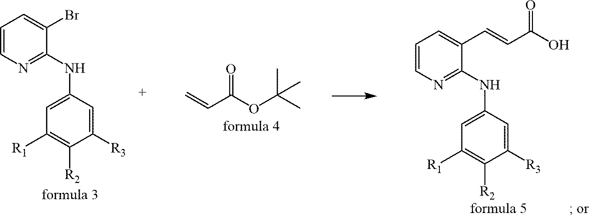

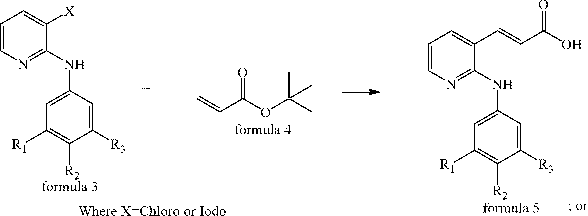

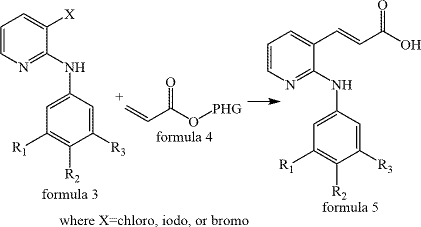

where PG represents any suitable protecting group known to the person of skill in the art having regard to the teachings herein (such as, but not limited to, an alkyl ester such methyl ester, ethyl ester, or t-butyl ester), and deprotecting includes removal of the PG protecting group to form a compound of formula 5 (deprotecting conditions may be selected based on the protecting group used - examples may include, for example, an acidic or basic hydrolysis to yield formula 5); and
reacting a compound of formula 5 with a compound of formula 6 to form an N-(2-aminophenyl)-prop-2-enamide derivative of formula 7:

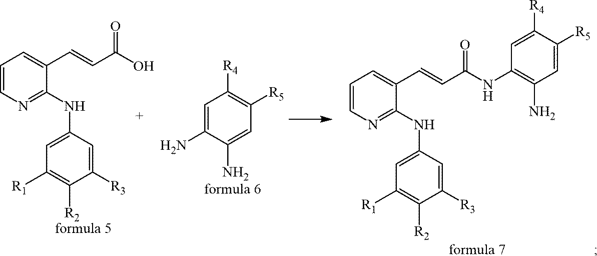

wherein
R₁, R₂, and R₃ are each independently selected from H, —OCH₃, —CF₃, Cl, or F;
R₄ is H, Cl, —OCH₃, —CH₃, or F; and
R₅ is H, Cl, —OCH₃, —CH₃, or F.

Embodiment 30: The method of Embodiment 29, wherein the compound of formula 1 is reacted with the compound of formula 2 in NaOBu-t, Pd(OAc)₂, PPh₃, and xylene.

Embodiment 31: The method of Embodiment 29 or 30, wherein the compound of formula 3 is reacted with the compound of formula 4 in Pd₂(dba)₃, DMF, and DIPEA.

Embodiment 32: The method of any one of Embodiments 29-31, wherein deprotection to form a compound of formula 5 is performed with TFA in DCM.

Embodiment 33: The method of any one of Embodiments 29-32, wherein the compound of formula 5 is reacted with the compound of formula 6 in BOP, Et₃ N, and MeCN.

Embodiment 34: A compound of formula 7 produced by a method according to any one of Embodiments 29-33.

Embodiment 35: The method of any one of Embodiments 29-33, wherein the compound of formula 7 is

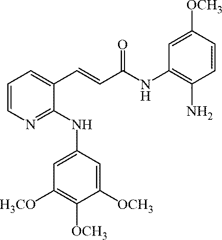 [SMILES: COc1ccc(N)c(NC(=O)/C=C/c2cccnc2Nc2cc(OC)c(OC)c(OC)c2)c1]

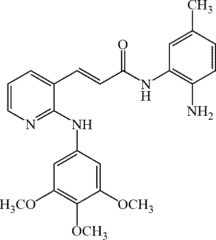 [SMILES: COc1cc(Nc2ncccc2/C=C/C(=O)Nc2cc(C)ccc2N)cc(OC)c1OC]

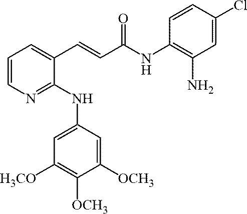 [SMILES: COc1cc(Nc2ncccc2/C=C/C(=O)Nc2ccc(Cl)cc2N)cc(OC)c1OC]

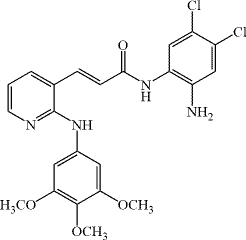 [SMILES: COc1cc(Nc2ncccc2/C=C/C(=O)Nc2cc(Cl)c(Cl)cc2N)cc(OC)c1OC]

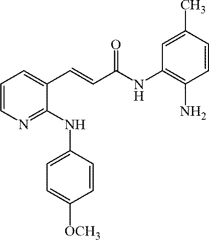 [SMILES: COc1ccc(Nc2ncccc2/C=C/C(=O)Nc2cc(C)ccc2N)cc1]

or

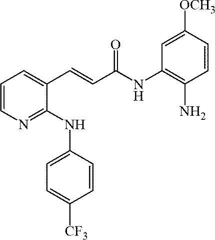 [SMILES: COc1ccc(N)c(NC(=O)/C=C/c2cccnc2Nc2ccc(C(F)(F)F)cc2)c1]

Embodiment 36: A compound which is any one of the following:

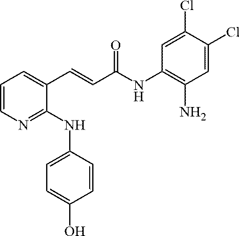 [SMILES: Nc1cc(Cl)c(Cl)cc1NC(=O)/C=C/c1cccnc1Nc1ccc(O)cc1]

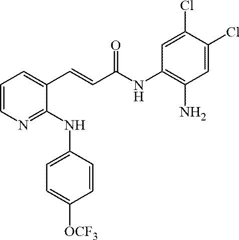 [SMILES: Nc1cc(Cl)c(Cl)cc1NC(=O)/C=C/c1cccnc1Nc1ccc(OC(F)(F)F)cc1]

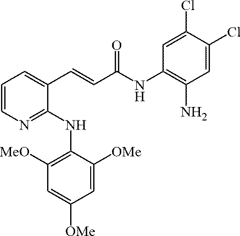 [SMILES: COc1cc(OC)c(Nc2ncccc2/C=C/C(=O)Nc2cc(Cl)c(Cl)cc2N)c(OC)c1]

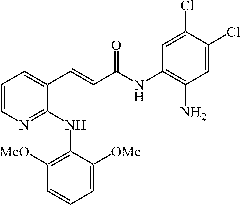 [SMILES: COc1cccc(OC)c1Nc1ncccc1/C=C/C(=O)Nc1cc(Cl)c(Cl)cc1N]

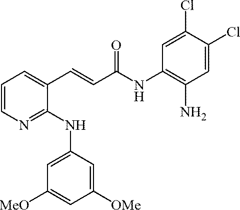 [SMILES: COc1cc(Nc2ncccc2/C=C/C(=O)Nc2cc(Cl)c(Cl)cc2N)cc(OC)c1]

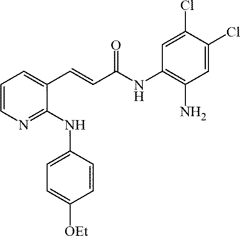 [SMILES: CCOc1ccc(Nc2ncccc2/C=C/C(=O)Nc2cc(Cl)c(Cl)cc2N)cc1]

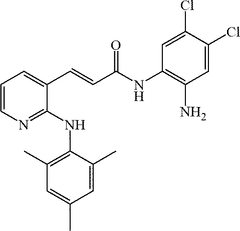 [SMILES: Cc1cc(C)c(Nc2ncccc2/C=C/C(=O)Nc2cc(Cl)c(Cl)cc2N)c(C)c1]

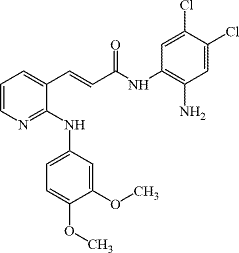 [SMILES: COc1ccc(Nc2ncccc2/C=C/C(=O)Nc2cc(Cl)c(Cl)cc2N)cc1OC]

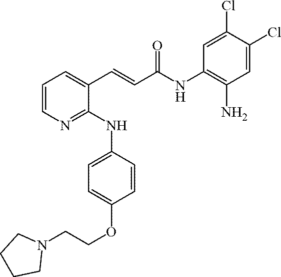 [SMILES: Nc1cc(Cl)c(Cl)cc1NC(=O)/C=C/c1cccnc1Nc1ccc(OCCN2CCCC2)cc1]

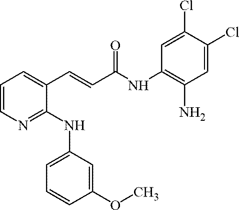 [SMILES: COc1cccc(Nc2ncccc2/C=C/C(=O)Nc2cc(Cl)c(Cl)cc2N)c1]

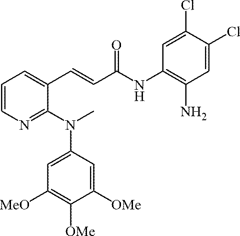 [SMILES: COc1cc(N(C)c2ncccc2/C=C/C(=O)Nc2cc(Cl)c(Cl)cc2N)cc(OC)c1OC]

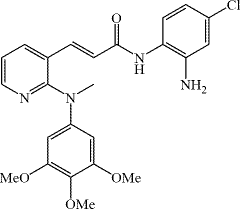 [SMILES: COc1cc(N(C)c2ncccc2/C=C/C(=O)Nc2ccc(Cl)cc2N)cc(OC)c1OC]

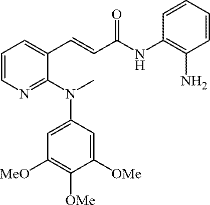 [SMILES: COc1cc(N(C)c2ncccc2/C=C/C(=O)Nc2ccccc2N)cc(OC)c1OC]

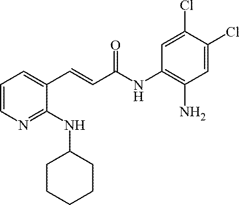 [SMILES: Nc1cc(Cl)c(Cl)cc1NC(=O)/C=C/c1cccnc1NC1CCCCC1]

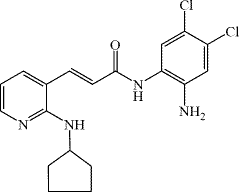 [SMILES: Nc1cc(Cl)c(Cl)cc1NC(=O)/C=C/c1cccnc1NC1CCCC1]

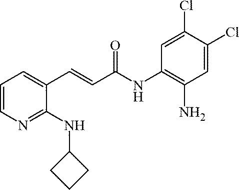 [SMILES: Nc1cc(Cl)c(Cl)cc1NC(=O)/C=C/c1cccnc1NC1CCC1]

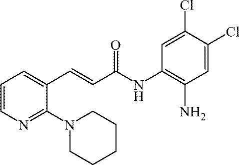 [SMILES: Nc1cc(Cl)c(Cl)cc1NC(=O)/C=C/c1cccnc1N1CCCCC1]

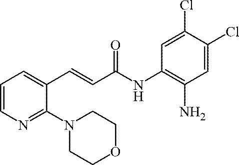 [SMILES: Nc1cc(Cl)c(Cl)cc1NC(=O)/C=C/c1cccnc1N1CCOCC1]

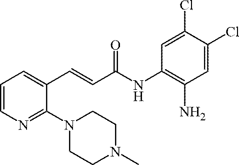 [SMILES: CN1CCN(c2ncccc2/C=C/C(=O)Nc2cc(Cl)c(Cl)cc2N)CC1]

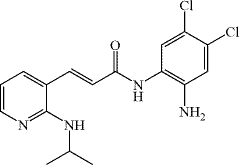 [SMILES: CC(C)Nc1ncccc1/C=C/C(=O)Nc1cc(Cl)c(Cl)cc1N]

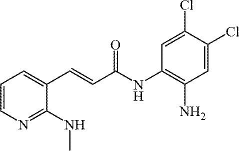 [SMILES: CNc1ncccc1/C=C/C(=O)Nc1cc(Cl)c(Cl)cc1N]

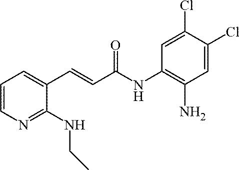 [SMILES: CCNc1ncccc1/C=C/C(=O)Nc1cc(Cl)c(Cl)cc1N]

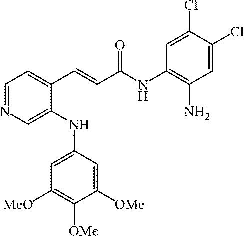 [SMILES: COc1cc(Nc2cnccc2/C=C/C(=O)Nc2cc(Cl)c(Cl)cc2N)cc(OC)c1OC]

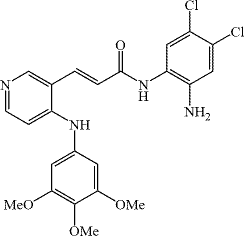 [SMILES: COc1cc(Nc2ccncc2/C=C/C(=O)Nc2cc(Cl)c(Cl)cc2N)cc(OC)c1OC]

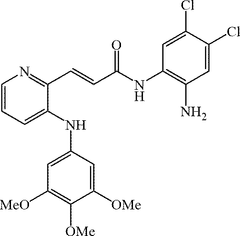 [SMILES: COc1cc(Nc2cccnc2/C=C/C(=O)Nc2cc(Cl)c(Cl)cc2N)cc(OC)c1OC]

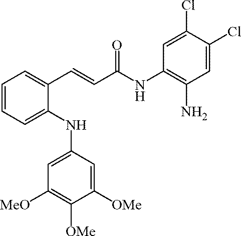 [SMILES: COc1cc(Nc2ccccc2/C=C/C(=O)Nc2cc(Cl)c(Cl)cc2N)cc(OC)c1OC]

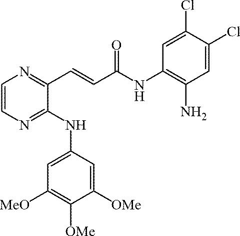 [SMILES: COc1cc(Nc2nccnc2/C=C/C(=O)Nc2cc(Cl)c(Cl)cc2N)cc(OC)c1OC]

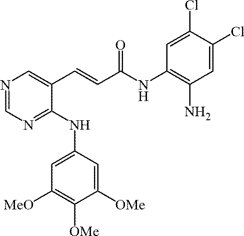 [SMILES: COc1cc(Nc2ncncc2/C=C/C(=O)Nc2cc(Cl)c(Cl)cc2N)cc(OC)c1OC]

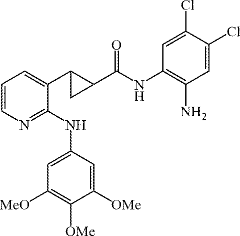 [SMILES: COc1cc(Nc2ncccc2C2CC2C(=O)Nc2cc(Cl)c(Cl)cc2N)cc(OC)c1OC]

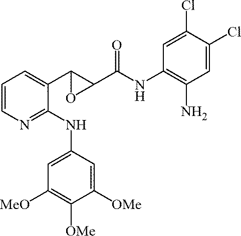 [SMILES: COc1cc(Nc2ncccc2C2OC2C(=O)Nc2cc(Cl)c(Cl)cc2N)cc(OC)c1OC]

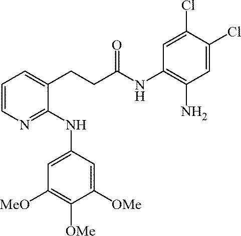 [SMILES: COc1cc(Nc2ncccc2CCC(=O)Nc2cc(Cl)c(Cl)cc2N)cc(OC)c1OC]

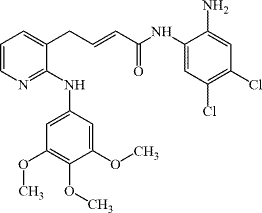 [SMILES: COc1cc(Nc2ncccc2C/C=C/C(=O)Nc2cc(Cl)c(Cl)cc2N)cc(OC)c1OC]

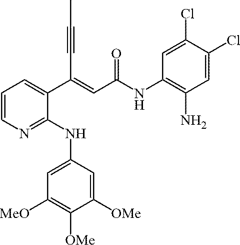 [SMILES: CC#C/C(=C\C(=O)Nc1cc(Cl)c(Cl)cc1N)c1cccnc1Nc1cc(OC)c(OC)c(OC)c1]

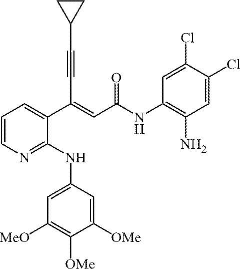 [SMILES: COc1cc(Nc2ncccc2/C(C#CC2CC2)=C/C(=O)Nc2cc(Cl)c(Cl)cc2N)cc(OC)c1OC]

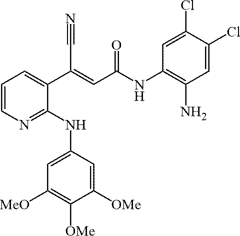 [SMILES: COc1cc(Nc2ncccc2/C(C#N)=C/C(=O)Nc2cc(Cl)c(Cl)cc2N)cc(OC)c1OC]

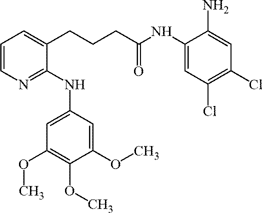 [SMILES: COc1cc(Nc2ncccc2CCCC(=O)Nc2cc(Cl)c(Cl)cc2N)cc(OC)c1OC]

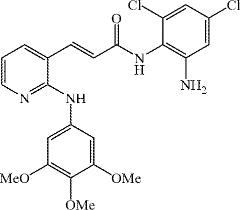 [SMILES: COc1cc(Nc2ncccc2/C=C/C(=O)Nc2c(N)cc(Cl)cc2Cl)cc(OC)c1OC]

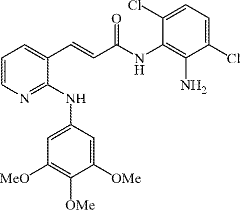 [SMILES: COc1cc(Nc2ncccc2/C=C/C(=O)Nc2c(Cl)ccc(Cl)c2N)cc(OC)c1OC]

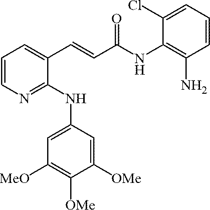 [SMILES: COc1cc(Nc2ncccc2/C=C/C(=O)Nc2c(N)cccc2Cl)cc(OC)c1OC]

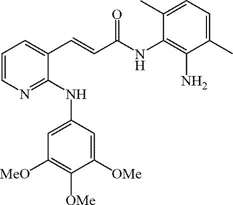 [SMILES: COc1cc(Nc2ncccc2/C=C/C(=O)Nc2c(C)ccc(C)c2N)cc(OC)c1OC]

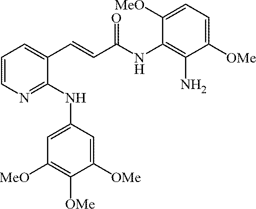 [SMILES: COc1ccc(OC)c(NC(=O)/C=C/c2cccnc2Nc2cc(OC)c(OC)c(OC)c2)c1N]

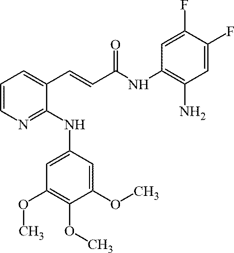 [SMILES: COc1cc(Nc2ncccc2/C=C/C(=O)Nc2cc(F)c(F)cc2N)cc(OC)c1OC]

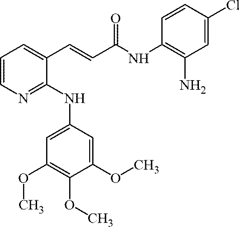 [SMILES: COc1cc(Nc2ncccc2/C=C/C(=O)Nc2ccc(Cl)cc2N)cc(OC)c1OC]

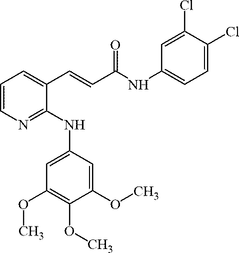 [SMILES: COc1cc(Nc2ncccc2/C=C/C(=O)Nc2ccc(Cl)c(Cl)c2)cc(OC)c1OC]

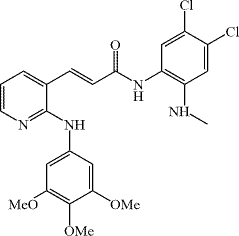 [SMILES: CNc1cc(Cl)c(Cl)cc1NC(=O)/C=C/c1cccnc1Nc1cc(OC)c(OC)c(OC)c1]

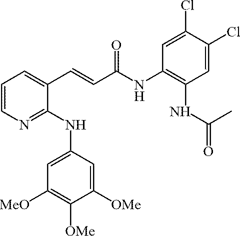 [SMILES: COc1cc(Nc2ncccc2/C=C/C(=O)Nc2cc(Cl)c(Cl)cc2NC(C)=O)cc(OC)c1OC]

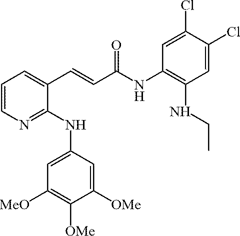 [SMILES: CCNc1cc(Cl)c(Cl)cc1NC(=O)/C=C/c1cccnc1Nc1cc(OC)c(OC)c(OC)c1]

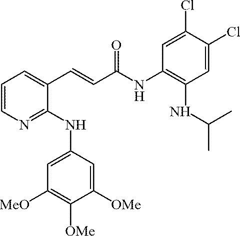 [SMILES: COc1cc(Nc2ncccc2/C=C/C(=O)Nc2cc(Cl)c(Cl)cc2NC(C)C)cc(OC)c1OC]

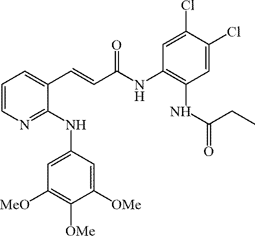 [SMILES: CCC(=O)Nc1cc(Cl)c(Cl)cc1NC(=O)/C=C/c1cccnc1Nc1cc(OC)c(OC)c(OC)c1]

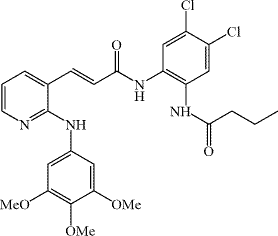 [SMILES: CCCC(=O)Nc1cc(Cl)c(Cl)cc1NC(=O)/C=C/c1cccnc1Nc1cc(OC)c(OC)c(OC)c1]

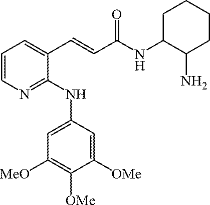 [SMILES: COc1cc(Nc2ncccc2/C=C/C(=O)NC2CCCCC2N)cc(OC)c1OC]

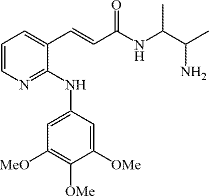 [SMILES: COc1cc(Nc2ncccc2/C=C/C(=O)NC(C)C(C)N)cc(OC)c1OC]

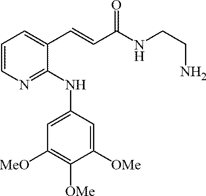 [SMILES: COc1cc(Nc2ncccc2/C=C/C(=O)NCCN)cc(OC)c1OC]

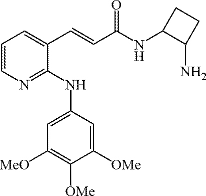 [SMILES: COc1cc(Nc2ncccc2/C=C/C(=O)NC2CCC2N)cc(OC)c1OC]

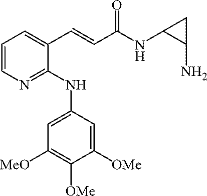 [SMILES: COc1cc(Nc2ncccc2/C=C/C(=O)NC2CC2N)cc(OC)c1OC]

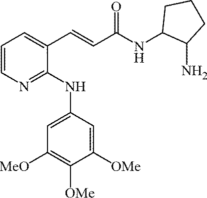 [SMILES: COc1cc(Nc2ncccc2/C=C/C(=O)NC2CCCC2N)cc(OC)c1OC]

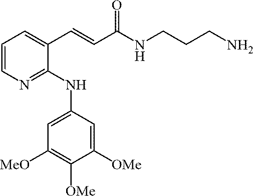 [SMILES: COc1cc(Nc2ncccc2/C=C/C(=O)NCCCN)cc(OC)c1OC]

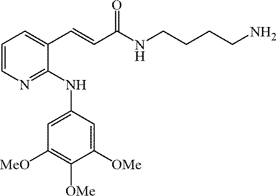 [SMILES: COc1cc(Nc2ncccc2/C=C/C(=O)NCCCCN)cc(OC)c1OC]

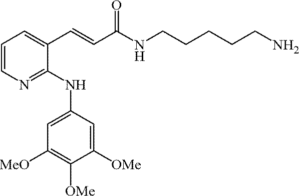 [SMILES: COc1cc(Nc2ncccc2/C=C/C(=O)NCCCCCN)cc(OC)c1OC]

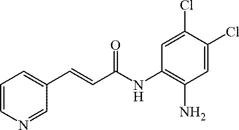 [SMILES: Nc1cc(Cl)c(Cl)cc1NC(=O)/C=C/c1cccnc1]

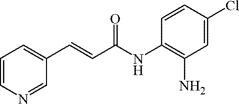 [SMILES: Nc1cc(Cl)ccc1NC(=O)/C=C/c1cccnc1]

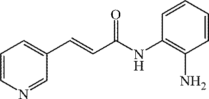 [SMILES: Nc1ccccc1NC(=O)/C=C/c1cccnc1]

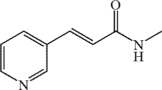

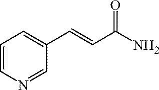 [SMILES: NC(=O)/C=C/c1cccnc1]

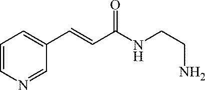 [SMILES: NCCNC(=O)/C=C/c1cccnc1]

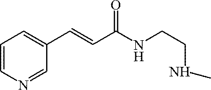

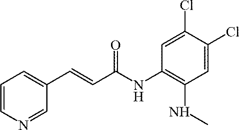 [SMILES: CNc1cc(Cl)c(Cl)cc1NC(=O)/C=C/c1cccnc1]

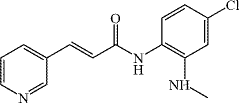 [SMILES: CNc1cc(Cl)ccc1NC(=O)/C=C/c1cccnc1]

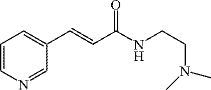 [SMILES: CN(C)CCNC(=O)/C=C/c1cccnc1]

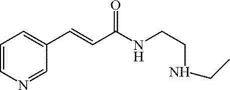 [SMILES: CCNCCNC(=O)/C=C/c1cccnc1]

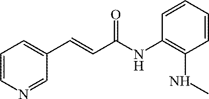 [SMILES: CNc1ccccc1NC(=O)/C=C/c1cccnc1]

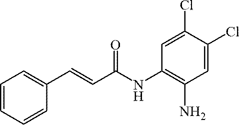 [SMILES: Nc1cc(Cl)c(Cl)cc1NC(=O)/C=C/c1ccccc1]

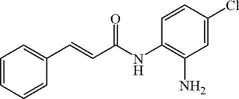 [SMILES: Nc1cc(Cl)ccc1NC(=O)/C=C/c1ccccc1]

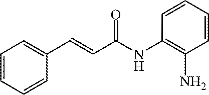 [SMILES: Nc1ccccc1NC(=O)/C=C/c1ccccc1]

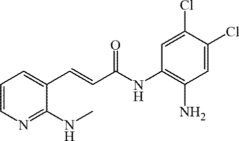 [SMILES: CNc1ncccc1/C=C/C(=O)Nc1cc(Cl)c(Cl)cc1N]

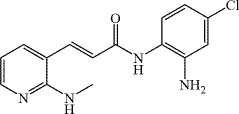 [SMILES: CNc1ncccc1/C=C/C(=O)Nc1ccc(Cl)cc1N]

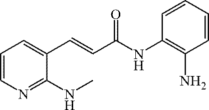 [SMILES: CNc1ncccc1/C=C/C(=O)Nc1ccccc1N]

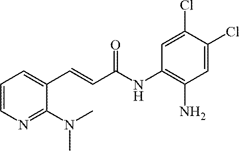 [SMILES: CN(C)c1ncccc1/C=C/C(=O)Nc1cc(Cl)c(Cl)cc1N]

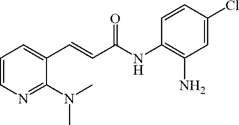 [SMILES: CN(C)c1ncccc1/C=C/C(=O)Nc1ccc(Cl)cc1N]

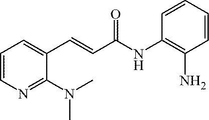 [SMILES: CN(C)c1ncccc1/C=C/C(=O)Nc1ccccc1N]

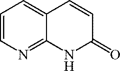 [SMILES: O=c1ccc2cccnc2[nH]1]

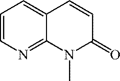

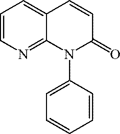 [SMILES: O=c1ccc2cccnc2n1-c1ccccc1]

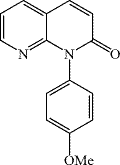 [SMILES: COc1ccc(-n2c(=O)ccc3cccnc32)cc1]

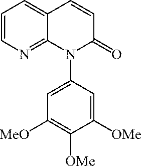 [SMILES: COc1cc(-n2c(=O)ccc3cccnc32)cc(OC)c1OC]

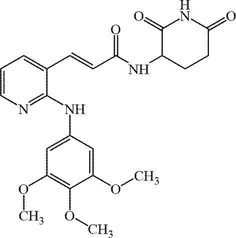 [SMILES: COc1cc(Nc2ncccc2/C=C/C(=O)NC2CCC(=O)NC2=O)cc(OC)c1OC]

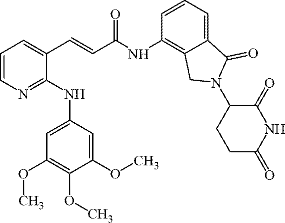 [SMILES: COc1cc(Nc2ncccc2/C=C/C(=O)Nc2cccc3c2CN(C2CCC(=O)NC2=O)C3=O)cc(OC)c1OC]

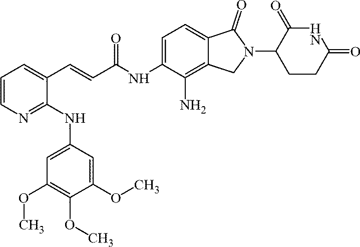

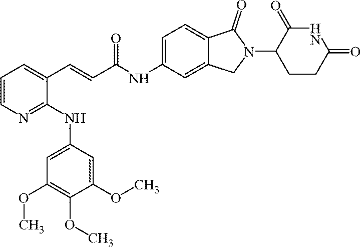 [SMILES: COc1cc(Nc2ncccc2/C=C/C(=O)Nc2ccc3c(c2)CN(C2CCC(=O)NC2=O)C3=O)cc(OC)c1OC]

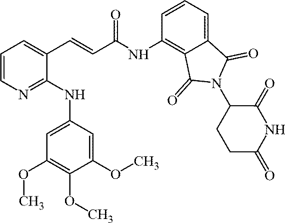 [SMILES: COc1cc(Nc2ncccc2/C=C/C(=O)Nc2cccc3c2C(=O)N(C2CCC(=O)NC2=O)C3=O)cc(OC)c1OC]

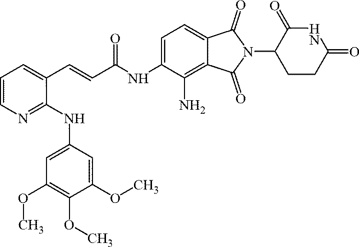 [SMILES: COc1cc(Nc2ncccc2/C=C/C(=O)Nc2ccc3c(c2N)C(=O)N(C2CCC(=O)NC2=O)C3=O)cc(OC)c1OC]

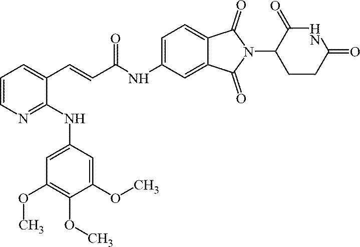 [SMILES: COc1cc(Nc2ncccc2/C=C/C(=O)Nc2ccc3c(c2)C(=O)N(C2CCC(=O)NC2=O)C3=O)cc(OC)c1OC]

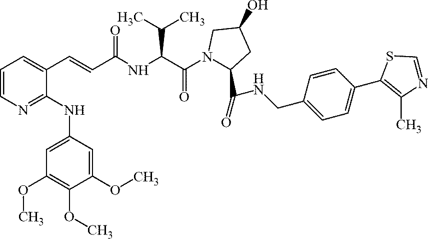 [SMILES: COc1cc(Nc2ncccc2/C=C/C(=O)N[C@H](C(=O)N2C[C@@H](O)C[C@H]2C(=O)NCc2ccc(-c3scnc3C)cc2)C(C)C)cc(OC)c1OC]

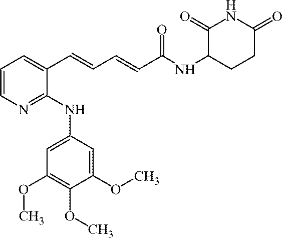 [SMILES: COc1cc(Nc2ncccc2/C=C/C=C/C(=O)NC2CCC(=O)NC2=O)cc(OC)c1OC]

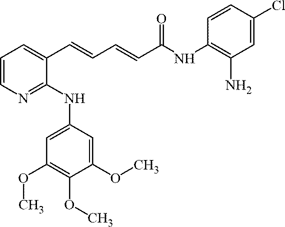 [SMILES: COc1cc(Nc2ncccc2/C=C/C=C/C(=O)Nc2ccc(Cl)cc2N)cc(OC)c1OC]

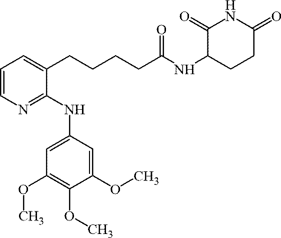 [SMILES: COc1cc(Nc2ncccc2CCCCC(=O)NC2CCC(=O)NC2=O)cc(OC)c1OC]

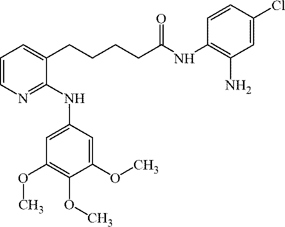 [SMILES: COc1cc(Nc2ncccc2CCCCC(=O)Nc2ccc(Cl)cc2N)cc(OC)c1OC]

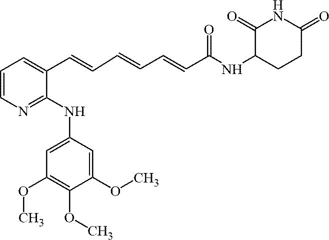 [SMILES: COc1cc(Nc2ncccc2/C=C/C=C/C=C/C(=O)NC2CCC(=O)NC2=O)cc(OC)c1OC]

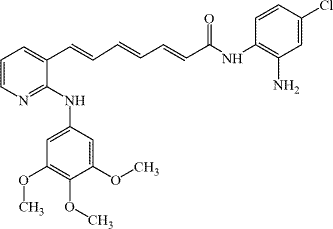 [SMILES: COc1cc(Nc2ncccc2/C=C/C=C/C=C/C(=O)Nc2ccc(Cl)cc2N)cc(OC)c1OC]

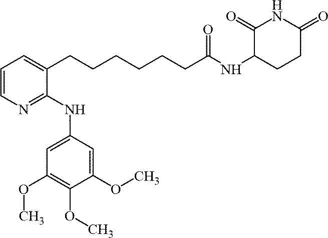 [SMILES: COc1cc(Nc2ncccc2CCCCCCC(=O)NC2CCC(=O)NC2=O)cc(OC)c1OC]

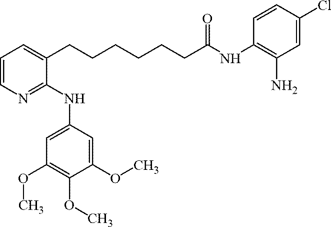 [SMILES: COc1cc(Nc2ncccc2CCCCCCC(=O)Nc2ccc(Cl)cc2N)cc(OC)c1OC]

One or more illustrative embodiments have been described by way of example. It will be understood to persons skilled in the art that a number of variations and modifications can be made without departing from the scope of the invention as defined in the claims.

### REFERENCES

1. Gao C1, Dimitrov T, Yong KJ, Tatetsu H, Jeong HW, Luo HR, Bradner JE, Tenen DG, Chai L. Targeting transcription factor SALL4 in acute myeloid leukemia by interrupting its interaction with an epigenetic complex. Blood. 2013 Feb 21;121(8):1413-21. doi: 10.1182/blood-2012-04-424275. Epub 2013 Jan 3.
2. Yong KJ, Li A, Ou WB, Hong CK, Zhao W, Wang F, Tatetsu H, Yan B, Qi L, Fletcher JA, Yang H, Soo R, Tenen DG, Chai L. Targeting SALL4 by entinostat in lung cancer. Oncotarget. 2016 Sep 26. doi: 10. 18632/oncotarget.12251.
3. Li A, Jiao Y, Yong KJ, Wang F, Gao C, Yan B, Srivastava S, Lim GS, Tang P, Yang H, Tenen DG, Chai L. SALL4 is a new target in endometrial cancer. Oncogene. 2015 Jan 2;34(1):63-72. doi: 10.1038/onc.2013.529.
4. Yong KJ, Gao C, Lim JS, Yan B, Yang H, Dimitrov T, Kawasaki A, Ong CW, Wong KF, Lee S, Ravikumar S, Srivastava S, Tian X, Poon RT, Fan ST, Luk JM, Dan YY, Salto-Tellez M, Chai L, Tenen DG. Oncofetal gene SALL4 in aggressive hepatocellular carcinoma. N Engl J Med. 2013 Jun 13;368(24):2266-76. doi: 10.1056/NEJMoa1300297.
5. Chai Li, Dimitrov Todor, Tenen D. G., Yong K. J. SALL4 And Uses Thereof. US 20150080315 A1.
6. A.V. Subba Rao, M.V.P.S. Vishnu Vardhan, N.V. Subba Reddy, T. Srinivasa Reddy, Siddiq Pasha Shaik, Chandrakant Bagul, Ahmed Kamal. Synthesis and biological evaluation of imidazopyridinyl-1.3,4-oxadiazole conjugates as apoptosis inducers and topoisomerase IIα inhibitors. Bioorganic Chemistry, Volume 69, December 2016, Pages 7-19
7. Ahmed Kamal, P.S. Srikanth, M.V.P.S. Vishnuvardhan, G. Bharath Kumar, Korrapati Suresh Babu, S.M. Ali Hussaini, Jeevak Sopanrao Kapure, Abdullah Alarifi. Combretastatin linked 1.3,4-oxadiazole conjugates as a Potent Tubulin Polymerization inhibitors. Bioorganic Chemistry, Volume 65, April 2016, Pages 126-136.
8. Ahmed Kamal, Anver Basha Shaik, Sowjanya Polepalli, G. Bharath Kumar, Vangala Santhosh Reddy, Rasala Mahesh, Srujana Garimella, Nishant Jain. Synthesis of arylpyrazole linked benzimidazole conjugates as potential microtubule disruptors. Bioorganic & Medicinal Chemistry, Volume 23, Issue 5, 1 Mar. 2015, Pages 1082-1095.
9. Ahmed Kamal, Md. Ashraf, Shaik Thokhir Basha, S.M. Ali Hussaini Shamshair Singh, M. V. P. S. Vishnuvardhan, B. Kiran and B. Sridhar. Design, synthesis and antiproliferative activity of the new conjugates of E7010 and resveratrol as tubulin polymerization inhibitors. Org. Biomol. Chem., 2016, 14, 1382-1394.

All references cited herein and elsewhere in the specification are herein incorporated by reference in their entireties.

1. A compound of Formula I: 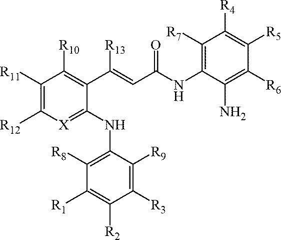
wherein
R₁ is H or —OCH₃;
R₂ is H, —OCH₃, —CF₃, or F;
R₃ is H or —OCH₃;
R₄ is H, Cl, —OCH₃, —CH₃, F, or —NH₂;
R₅ is H, Cl, —OCH₃, —CH₃, F, or —NH₂;
R₆ is H, Cl, —OCH₃, or —CH₃;
R₇ is H, Cl, —OCH₃, or —CH₃;
R₈ is H, —OCH₃, or —CH₃;
R₉ is H, —OCH₃, or —CH₃;
each of R₁₀, R₁₁, and R₁₂ is H, or R₁₀ and R₁₁ together with the carbon atoms to which they are attached form a 5 or 6-membered aryl or heteroaryl ring and R₁₂ is H, or R₁₁ and R₁₂ together with the carbon atoms to which they are attached form a 5 or 6-membered aryl or heteroaryl ring and R₁₀is H;
R₁₃ is H, —C≡C—CH₃, —C≡C—C₃H₅, or —C═N; and
X is N or CH.

2. The compound of claim 1, wherein R₁ is —OCH₃.

3. The compound of claim 1, wherein R₂ is —OCH₃ or —CF₃.

4. The compound of claim 1, wherein R₃ is —OCH₃.

5. The compound of claim 1, wherein R₄ is H, Cl, —OCH₃, or —CH₃.

6. The compound of claim 1, wherein R₅ is H or Cl.

7. The compound of claim 1, wherein R₆ is H.

8. The compound of claim 1, wherein R₇ is H.

9. The compound of claim 1, wherein R₈ is H.

10. The compound of claim 1, wherein R₉ is H.

11. The compound of claim 1, wherein R₁₀, R₁₁, and R₁₂ are each H.

12. The compound of claim 1, wherein R₁₃ is H.

13. The compound of claim 1, wherein X is N.

14. The compound of claim 1, wherein the compound is: 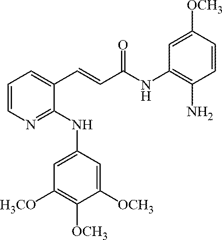 [SMILES: COc1ccc(N)c(NC(=O)/C=C/c2cccnc2Nc2cc(OC)c(OC)c(OC)c2)c1] 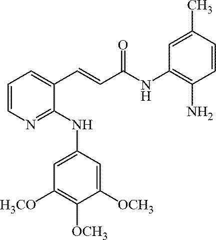 [SMILES: COc1cc(Nc2ncccc2/C=C/C(=O)Nc2cc(C)ccc2N)cc(OC)c1OC] 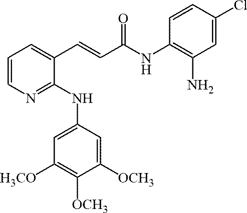 [SMILES: COc1cc(Nc2ncccc2/C=C/C(=O)Nc2ccc(Cl)cc2N)cc(OC)c1OC] 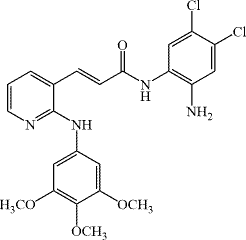 [SMILES: COc1cc(Nc2ncccc2/C=C/C(=O)Nc2cc(Cl)c(Cl)cc2N)cc(OC)c1OC] 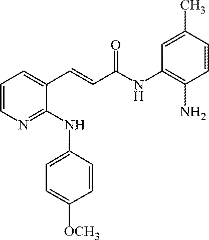 [SMILES: COc1ccc(Nc2ncccc2/C=C/C(=O)Nc2cc(C)ccc2N)cc1]
or 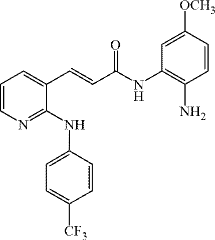 [SMILES: COc1ccc(N)c(NC(=O)/C=C/c2cccnc2Nc2ccc(C(F)(F)F)cc2)c1]
.

15. The compound of claim 14, wherein the compound is: 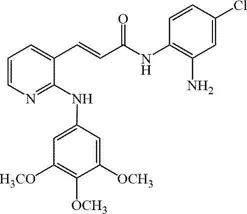 [SMILES: COc1cc(Nc2ncccc2/C=C/C(=O)Nc2ccc(Cl)cc2N)cc(OC)c1OC]
.

16. A composition comprising the compound of claim 1, and a pharmaceutically acceptable carrier, excipient, or diluent.

17. A method for treating cancer in a cell or subject in need thereof, said method comprising:
administering a compound of claim 1 to the cell or subject.

18. (canceled)

19. (canceled)

20. The method of claim 17, wherein the cancer is a SALL4-expressing cancer.

21. The method of claim 20, wherein the cancer is a SALL4-expressing cancer having a high level of SALL4 expression.

22. The method of claim 17, wherein the cancer is lung cancer, liver cancer, or breast cancer.

23. The method of claim 22, wherein the cancer is NSCLC cancer, cervical cancer, or germ cell cancer.

24. A method for preparing an N-(2-aminophenyl)-prop-2-enamide derivative, said method comprising:
reacting a compound of formula 1 with a compound of formula 2 to form a compound of formula 3: 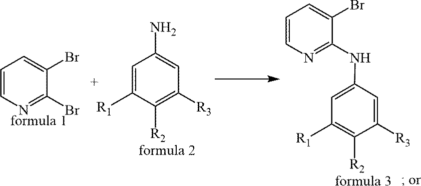 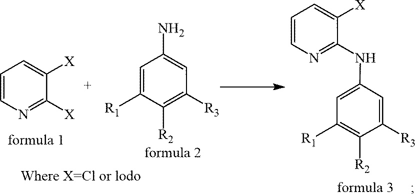
reacting the compound of formula 3 with a compound of formula 4, and deprotecting, to form a compound of formula 5: 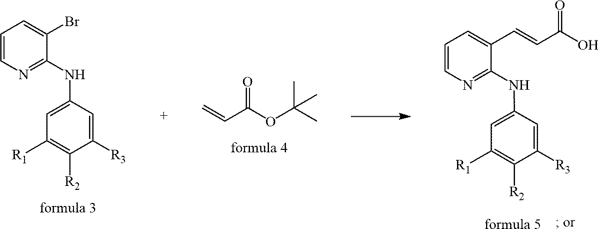 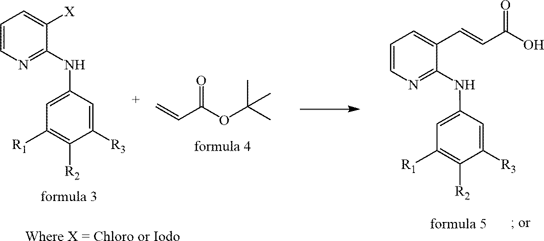 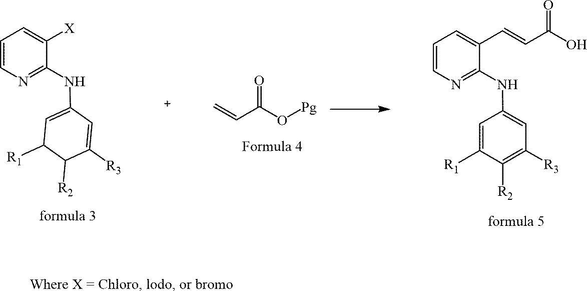
wherein PG represents a protecting group, and deprotecting includes removal of the PG protecting group to form a compound of formula 5;
and
reacting a compound of formula 5 with a compound of formula 6 to form an N-(2-aminophenyl)-prop-2-enamide derivative of formula 7: 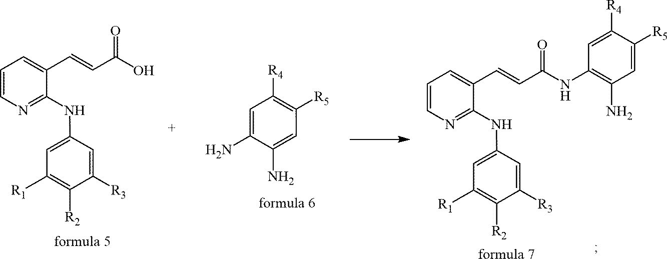
wherein
R₁, R₂, and R₃ are each independently selected from H, —OCH₃, —CF₃, Cl, or F;
R₄ is H, Cl, —OCH₃, —CH₃, or F; and
R₅ is H, Cl, —OCH₃, —CH₃, or F.

25. A compound which is any one of the following: 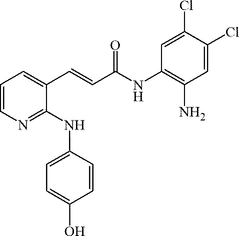 [SMILES: Nc1cc(Cl)c(Cl)cc1NC(=O)/C=C/c1cccnc1Nc1ccc(O)cc1] 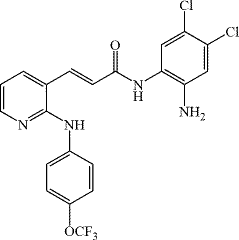 [SMILES: Nc1cc(Cl)c(Cl)cc1NC(=O)/C=C/c1cccnc1Nc1ccc(OC(F)(F)F)cc1] 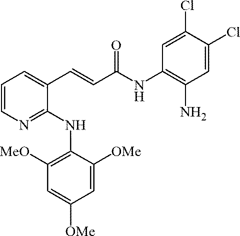 [SMILES: COc1cc(OC)c(Nc2ncccc2/C=C/C(=O)Nc2cc(Cl)c(Cl)cc2N)c(OC)c1] 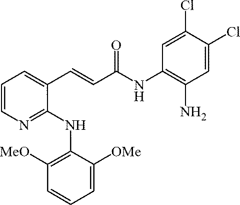 [SMILES: COc1cccc(OC)c1Nc1ncccc1/C=C/C(=O)Nc1cc(Cl)c(Cl)cc1N] 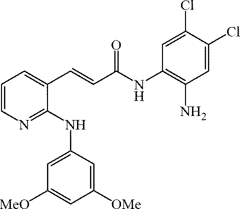 [SMILES: COc1cc(Nc2ncccc2/C=C/C(=O)Nc2cc(Cl)c(Cl)cc2N)cc(OC)c1] 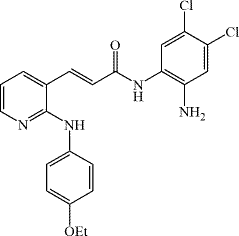 [SMILES: CCOc1ccc(Nc2ncccc2/C=C/C(=O)Nc2cc(Cl)c(Cl)cc2N)cc1] 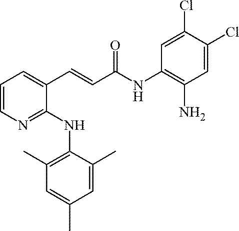 [SMILES: Cc1cc(C)c(Nc2ncccc2/C=C/C(=O)Nc2cc(Cl)c(Cl)cc2N)c(C)c1] 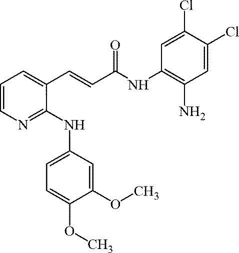 [SMILES: COc1ccc(Nc2ncccc2/C=C/C(=O)Nc2cc(Cl)c(Cl)cc2N)cc1OC] 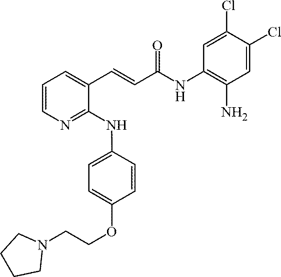 [SMILES: Nc1cc(Cl)c(Cl)cc1NC(=O)/C=C/c1cccnc1Nc1ccc(OCCN2CCCC2)cc1] 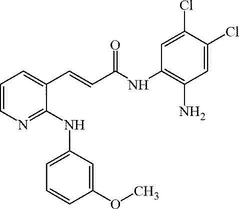 [SMILES: COc1cccc(Nc2ncccc2/C=C/C(=O)Nc2cc(Cl)c(Cl)cc2N)c1] 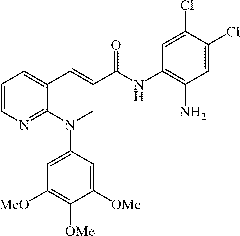 [SMILES: COc1cc(N(C)c2ncccc2/C=C/C(=O)Nc2cc(Cl)c(Cl)cc2N)cc(OC)c1OC] 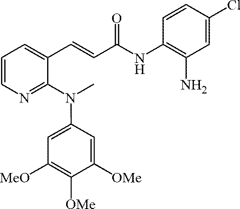 [SMILES: COc1cc(N(C)c2ncccc2/C=C/C(=O)Nc2ccc(Cl)cc2N)cc(OC)c1OC] 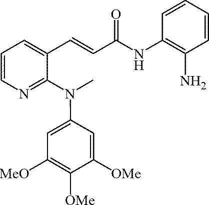 [SMILES: COc1cc(N(C)c2ncccc2/C=C/C(=O)Nc2ccccc2N)cc(OC)c1OC] 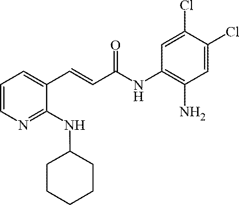 [SMILES: Nc1cc(Cl)c(Cl)cc1NC(=O)/C=C/c1cccnc1NC1CCCCC1] 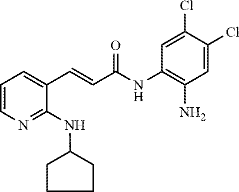 [SMILES: Nc1cc(Cl)c(Cl)cc1NC(=O)/C=C/c1cccnc1NC1CCCC1] 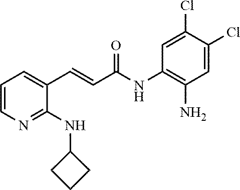 [SMILES: Nc1cc(Cl)c(Cl)cc1NC(=O)/C=C/c1cccnc1NC1CCC1] 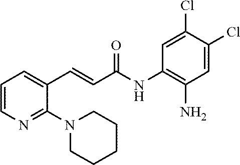 [SMILES: Nc1cc(Cl)c(Cl)cc1NC(=O)/C=C/c1cccnc1N1CCCCC1] 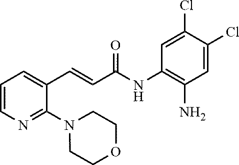 [SMILES: Nc1cc(Cl)c(Cl)cc1NC(=O)/C=C/c1cccnc1N1CCOCC1] 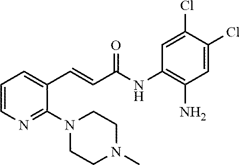 [SMILES: CN1CCN(c2ncccc2/C=C/C(=O)Nc2cc(Cl)c(Cl)cc2N)CC1] 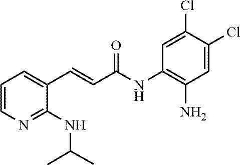 [SMILES: CC(C)Nc1ncccc1/C=C/C(=O)Nc1cc(Cl)c(Cl)cc1N] 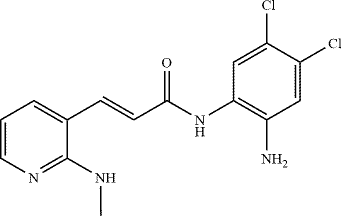 [SMILES: CNc1ncccc1/C=C/C(=O)Nc1cc(Cl)c(Cl)cc1N] 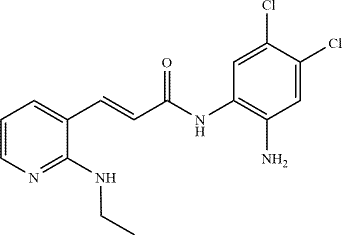 [SMILES: CCNc1ncccc1/C=C/C(=O)Nc1cc(Cl)c(Cl)cc1N] 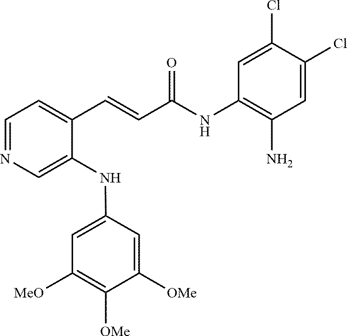 [SMILES: COc1cc(Nc2cnccc2/C=C/C(=O)Nc2cc(Cl)c(Cl)cc2N)cc(OC)c1OC] 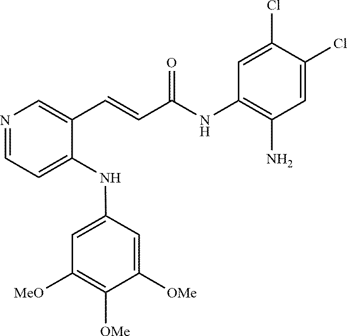 [SMILES: COc1cc(Nc2ccncc2/C=C/C(=O)Nc2cc(Cl)c(Cl)cc2N)cc(OC)c1OC] 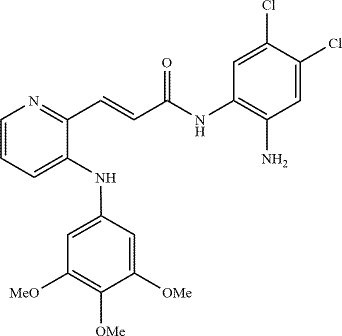 [SMILES: COc1cc(Nc2cccnc2/C=C/C(=O)Nc2cc(Cl)c(Cl)cc2N)cc(OC)c1OC] 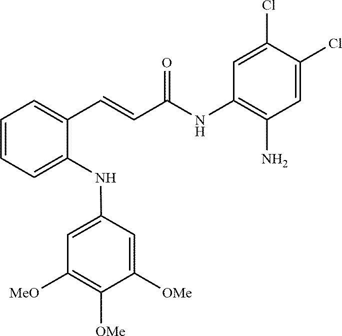 [SMILES: COc1cc(Nc2ccccc2/C=C/C(=O)Nc2cc(Cl)c(Cl)cc2N)cc(OC)c1OC] 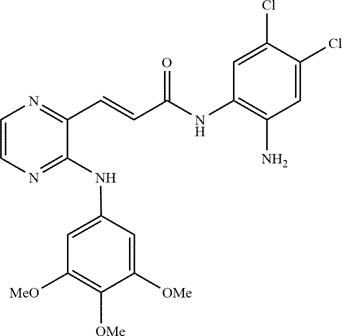 [SMILES: COc1cc(Nc2nccnc2/C=C/C(=O)Nc2cc(Cl)c(Cl)cc2N)cc(OC)c1OC] 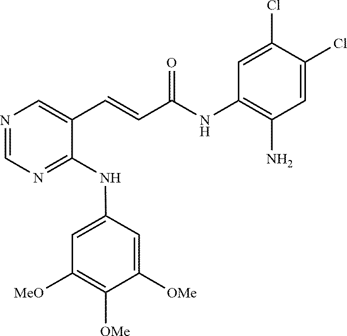 [SMILES: COc1cc(Nc2ncncc2/C=C/C(=O)Nc2cc(Cl)c(Cl)cc2N)cc(OC)c1OC] 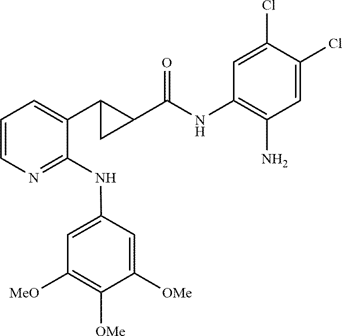 [SMILES: COc1cc(Nc2ncccc2C2CC2C(=O)Nc2cc(Cl)c(Cl)cc2N)cc(OC)c1OC] 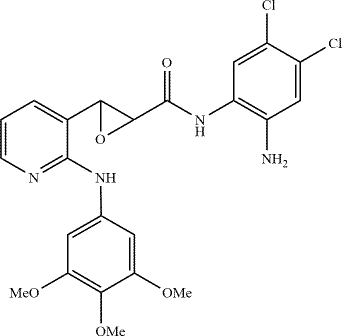 [SMILES: COc1cc(Nc2ncccc2C2OC2C(=O)Nc2cc(Cl)c(Cl)cc2N)cc(OC)c1OC] 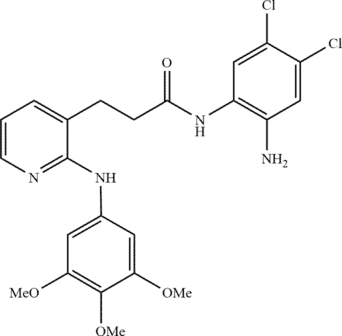 [SMILES: COc1cc(Nc2ncccc2CCC(=O)Nc2cc(Cl)c(Cl)cc2N)cc(OC)c1OC] 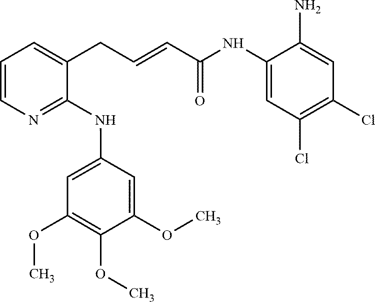 [SMILES: COc1cc(Nc2ncccc2C/C=C/C(=O)Nc2cc(Cl)c(Cl)cc2N)cc(OC)c1OC] 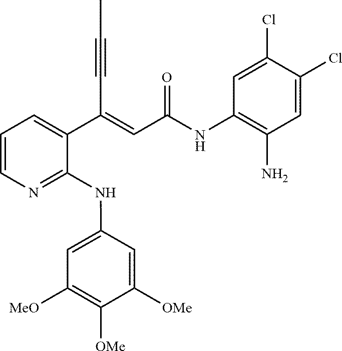 [SMILES: CC#C/C(=C\C(=O)Nc1cc(Cl)c(Cl)cc1N)c1cccnc1Nc1cc(OC)c(OC)c(OC)c1] 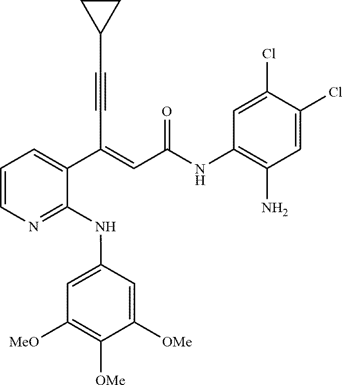 [SMILES: COc1cc(Nc2ncccc2/C(C#CC2CC2)=C/C(=O)Nc2cc(Cl)c(Cl)cc2N)cc(OC)c1OC] 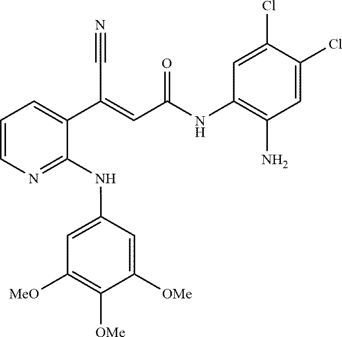 [SMILES: COc1cc(Nc2ncccc2/C(C#N)=C/C(=O)Nc2cc(Cl)c(Cl)cc2N)cc(OC)c1OC] 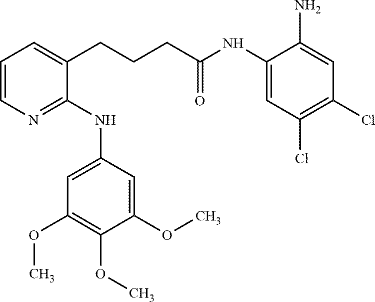 [SMILES: COc1cc(Nc2ncccc2CCCC(=O)Nc2cc(Cl)c(Cl)cc2N)cc(OC)c1OC] 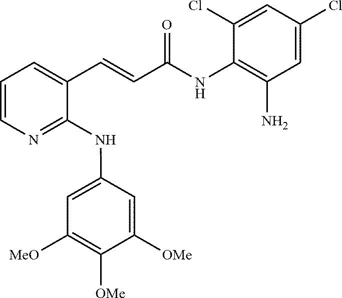 [SMILES: COc1cc(Nc2ncccc2/C=C/C(=O)Nc2c(N)cc(Cl)cc2Cl)cc(OC)c1OC] 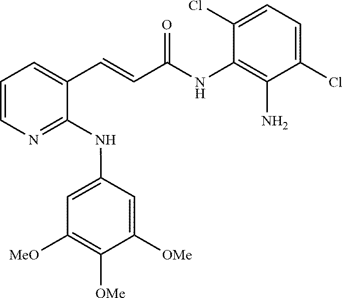 [SMILES: COc1cc(Nc2ncccc2/C=C/C(=O)Nc2c(Cl)ccc(Cl)c2N)cc(OC)c1OC] 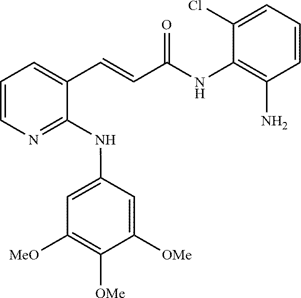 [SMILES: COc1cc(Nc2ncccc2/C=C/C(=O)Nc2c(N)cccc2Cl)cc(OC)c1OC] 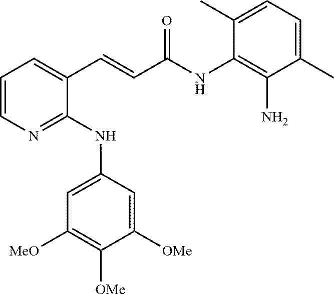 [SMILES: COc1cc(Nc2ncccc2/C=C/C(=O)Nc2c(C)ccc(C)c2N)cc(OC)c1OC] 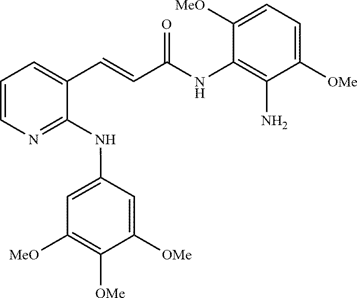 [SMILES: COc1ccc(OC)c(NC(=O)/C=C/c2cccnc2Nc2cc(OC)c(OC)c(OC)c2)c1N] 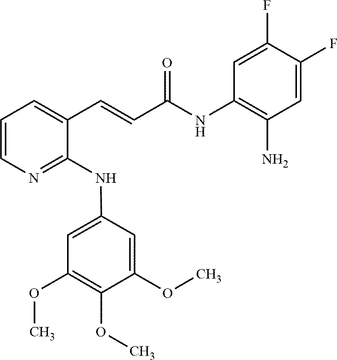 [SMILES: COc1cc(Nc2ncccc2/C=C/C(=O)Nc2cc(F)c(F)cc2N)cc(OC)c1OC] 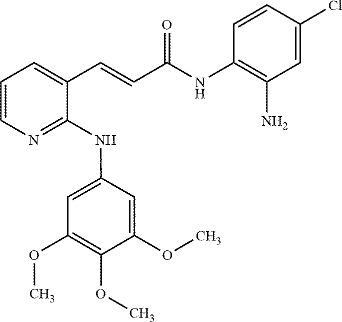 [SMILES: COc1cc(Nc2ncccc2/C=C/C(=O)Nc2ccc(Cl)cc2N)cc(OC)c1OC] 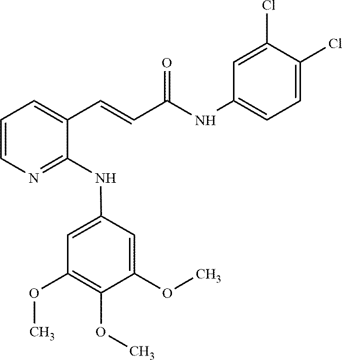 [SMILES: COc1cc(Nc2ncccc2/C=C/C(=O)Nc2ccc(Cl)c(Cl)c2)cc(OC)c1OC] 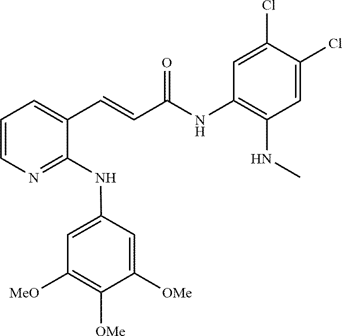 [SMILES: CNc1cc(Cl)c(Cl)cc1NC(=O)/C=C/c1cccnc1Nc1cc(OC)c(OC)c(OC)c1] 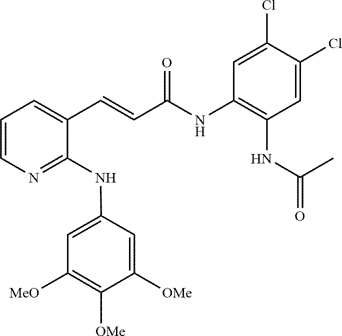 [SMILES: COc1cc(Nc2ncccc2/C=C/C(=O)Nc2cc(Cl)c(Cl)cc2NC(C)=O)cc(OC)c1OC] 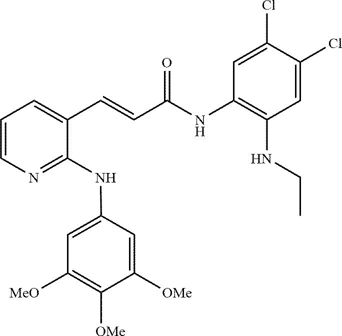 [SMILES: CCNc1cc(Cl)c(Cl)cc1NC(=O)/C=C/c1cccnc1Nc1cc(OC)c(OC)c(OC)c1] 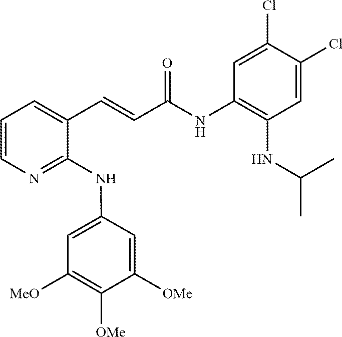 [SMILES: COc1cc(Nc2ncccc2/C=C/C(=O)Nc2cc(Cl)c(Cl)cc2NC(C)C)cc(OC)c1OC] 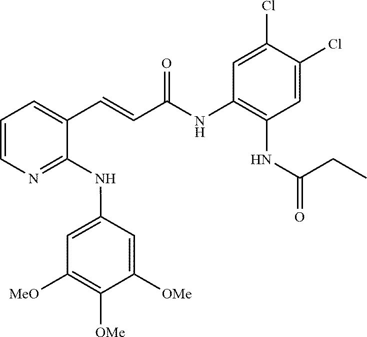 [SMILES: CCC(=O)Nc1cc(Cl)c(Cl)cc1NC(=O)/C=C/c1cccnc1Nc1cc(OC)c(OC)c(OC)c1] 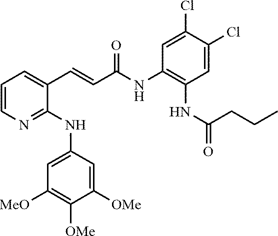 [SMILES: CCCC(=O)Nc1cc(Cl)c(Cl)cc1NC(=O)/C=C/c1cccnc1Nc1cc(OC)c(OC)c(OC)c1] 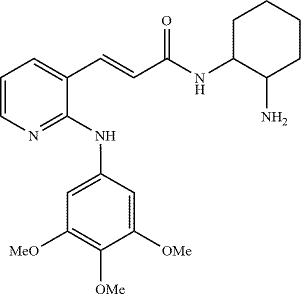 [SMILES: COc1cc(Nc2ncccc2/C=C/C(=O)NC2CCCCC2N)cc(OC)c1OC] 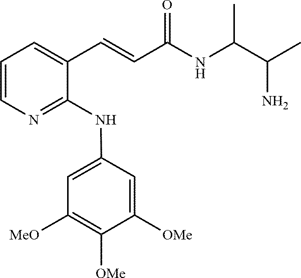 [SMILES: COc1cc(Nc2ncccc2/C=C/C(=O)NC(C)C(C)N)cc(OC)c1OC] 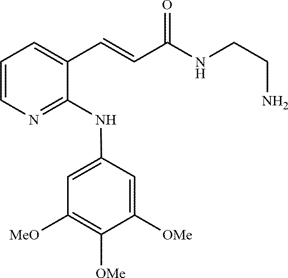 [SMILES: COc1cc(Nc2ncccc2/C=C/C(=O)NCCN)cc(OC)c1OC] 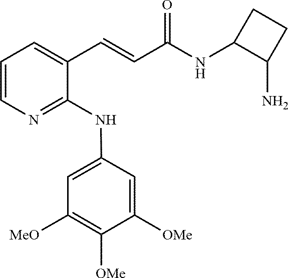 [SMILES: COc1cc(Nc2ncccc2/C=C/C(=O)NC2CCC2N)cc(OC)c1OC] 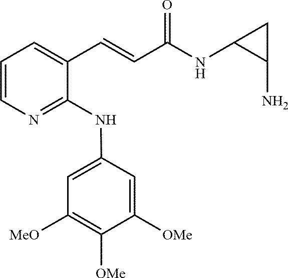 [SMILES: COc1cc(Nc2ncccc2/C=C/C(=O)NC2CC2N)cc(OC)c1OC] 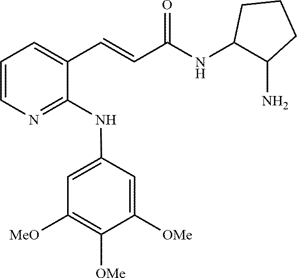 [SMILES: COc1cc(Nc2ncccc2/C=C/C(=O)NC2CCCC2N)cc(OC)c1OC] 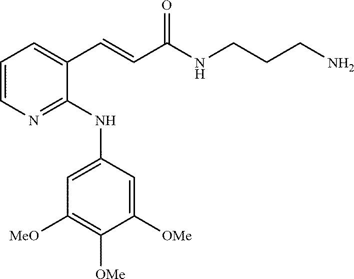 [SMILES: COc1cc(Nc2ncccc2/C=C/C(=O)NCCCN)cc(OC)c1OC] 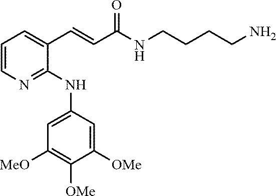 [SMILES: COc1cc(Nc2ncccc2/C=C/C(=O)NCCCCN)cc(OC)c1OC] 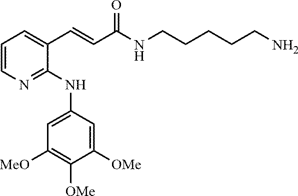 [SMILES: COc1cc(Nc2ncccc2/C=C/C(=O)NCCCCCN)cc(OC)c1OC] 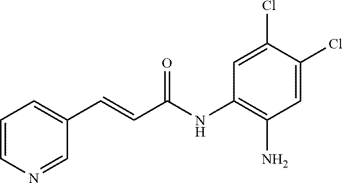 [SMILES: Nc1cc(Cl)c(Cl)cc1NC(=O)/C=C/c1cccnc1] 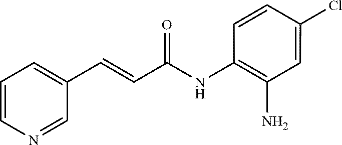 [SMILES: Nc1cc(Cl)ccc1NC(=O)/C=C/c1cccnc1] 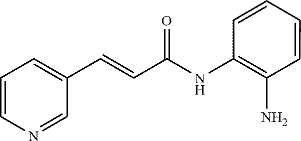 [SMILES: Nc1ccccc1NC(=O)/C=C/c1cccnc1] 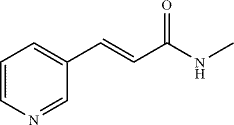 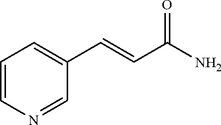 [SMILES: NC(=O)/C=C/c1cccnc1] 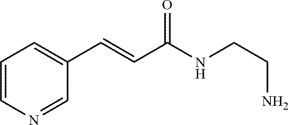 [SMILES: NCCNC(=O)/C=C/c1cccnc1] 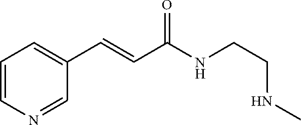 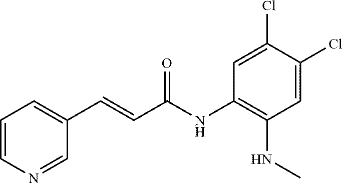 [SMILES: CNc1cc(Cl)c(Cl)cc1NC(=O)/C=C/c1cccnc1] 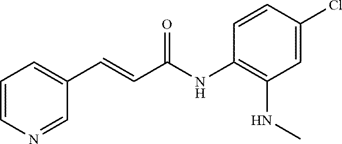 [SMILES: CNc1cc(Cl)ccc1NC(=O)/C=C/c1cccnc1] 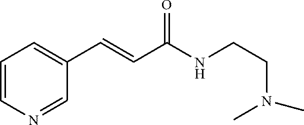 [SMILES: CN(C)CCNC(=O)/C=C/c1cccnc1] 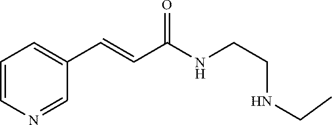 [SMILES: CCNCCNC(=O)/C=C/c1cccnc1] 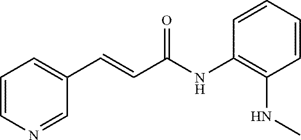 [SMILES: CNc1ccccc1NC(=O)/C=C/c1cccnc1] 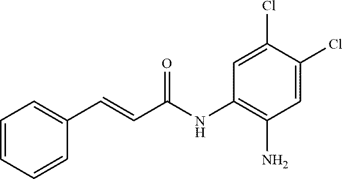 [SMILES: Nc1cc(Cl)c(Cl)cc1NC(=O)/C=C/c1ccccc1] 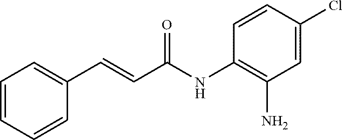 [SMILES: Nc1cc(Cl)ccc1NC(=O)/C=C/c1ccccc1] 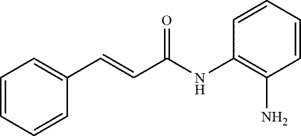 [SMILES: Nc1ccccc1NC(=O)/C=C/c1ccccc1] 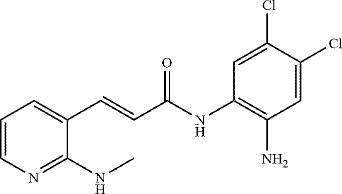 [SMILES: CNc1ncccc1/C=C/C(=O)Nc1cc(Cl)c(Cl)cc1N] 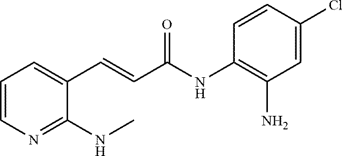 [SMILES: CNc1ncccc1/C=C/C(=O)Nc1ccc(Cl)cc1N] 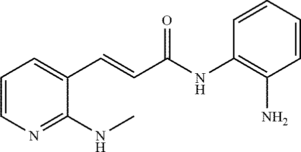 [SMILES: CNc1ncccc1/C=C/C(=O)Nc1ccccc1N] 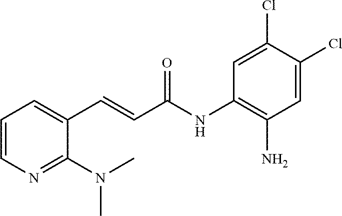 [SMILES: CN(C)c1ncccc1/C=C/C(=O)Nc1cc(Cl)c(Cl)cc1N] 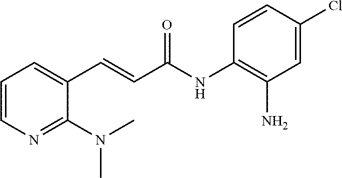 [SMILES: CN(C)c1ncccc1/C=C/C(=O)Nc1ccc(Cl)cc1N] 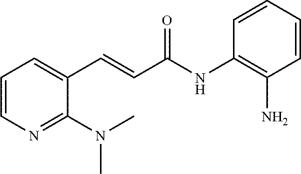 [SMILES: CN(C)c1ncccc1/C=C/C(=O)Nc1ccccc1N] 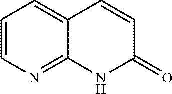 [SMILES: O=c1ccc2cccnc2[nH]1] 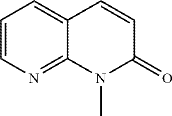 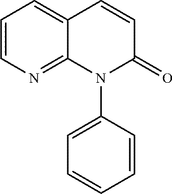 [SMILES: O=c1ccc2cccnc2n1-c1ccccc1] 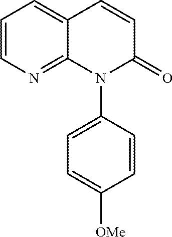 [SMILES: COc1ccc(-n2c(=O)ccc3cccnc32)cc1] 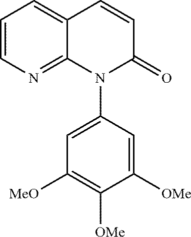 [SMILES: COc1cc(-n2c(=O)ccc3cccnc32)cc(OC)c1OC] 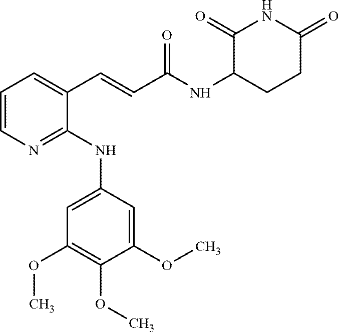 [SMILES: COc1cc(Nc2ncccc2/C=C/C(=O)NC2CCC(=O)NC2=O)cc(OC)c1OC] 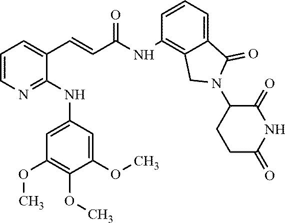 [SMILES: COc1cc(Nc2ncccc2/C=C/C(=O)Nc2cccc3c2CN(C2CCC(=O)NC2=O)C3=O)cc(OC)c1OC] 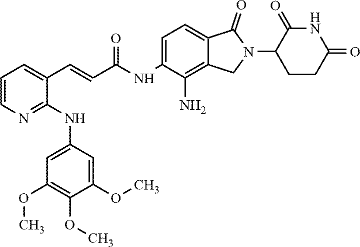 [SMILES: COc1cc(Nc2ncccc2/C=C/C(=O)Nc2ccc3c(c2N)CN(C2CCC(=O)NC2=O)C3=O)cc(OC)c1OC] 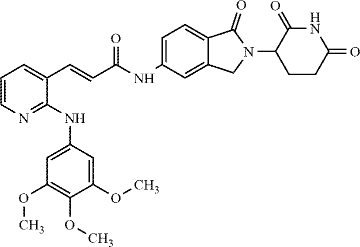 [SMILES: COc1cc(Nc2ncccc2/C=C/C(=O)Nc2ccc3c(c2)CN(C2CCC(=O)NC2=O)C3=O)cc(OC)c1OC] 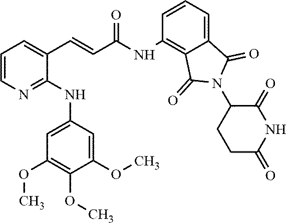 [SMILES: COc1cc(Nc2ncccc2/C=C/C(=O)Nc2cccc3c2C(=O)N(C2CCC(=O)NC2=O)C3=O)cc(OC)c1OC] 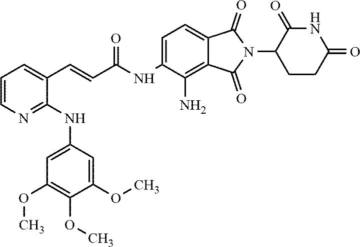 [SMILES: COc1cc(Nc2ncccc2/C=C/C(=O)Nc2ccc3c(c2N)C(=O)N(C2CCC(=O)NC2=O)C3=O)cc(OC)c1OC] 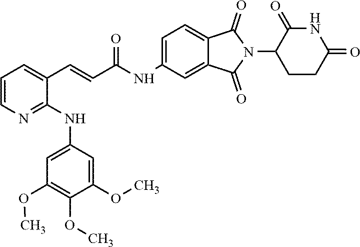 [SMILES: COc1cc(Nc2ncccc2/C=C/C(=O)Nc2ccc3c(c2)C(=O)N(C2CCC(=O)NC2=O)C3=O)cc(OC)c1OC] 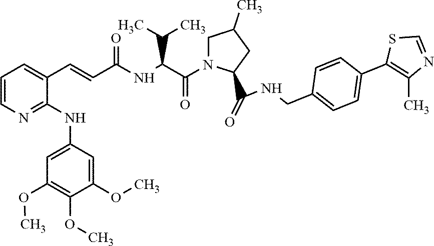 [SMILES: COc1cc(Nc2ncccc2/C=C/C(=O)N[C@H](C(=O)N2CC(C)C[C@H]2C(=O)NCc2ccc(-c3scnc3C)cc2)C(C)C)cc(OC)c1OC] 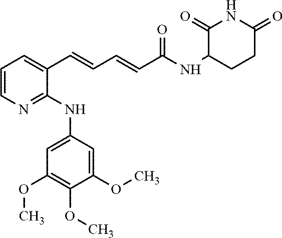 [SMILES: COc1cc(Nc2ncccc2/C=C/C=C/C(=O)NC2CCC(=O)NC2=O)cc(OC)c1OC] 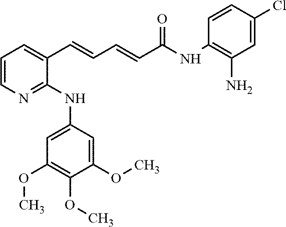 [SMILES: COc1cc(Nc2ncccc2/C=C/C=C/C(=O)Nc2ccc(Cl)cc2N)cc(OC)c1OC] 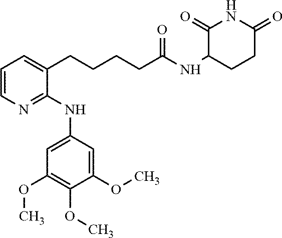 [SMILES: COc1cc(Nc2ncccc2CCCCC(=O)NC2CCC(=O)NC2=O)cc(OC)c1OC] 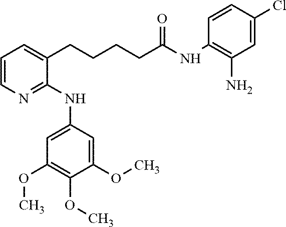 [SMILES: COc1cc(Nc2ncccc2CCCCC(=O)Nc2ccc(Cl)cc2N)cc(OC)c1OC] 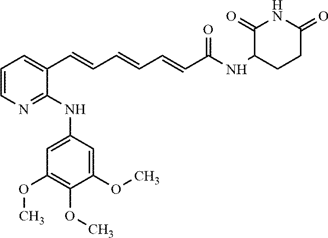 [SMILES: COc1cc(Nc2ncccc2/C=C/C=C/C=C/C(=O)NC2CCC(=O)NC2=O)cc(OC)c1OC] 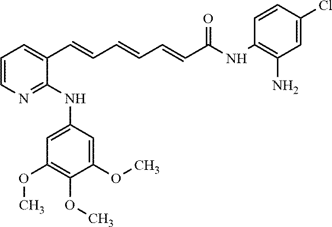 [SMILES: COc1cc(Nc2ncccc2/C=C/C=C/C=C/C(=O)Nc2ccc(Cl)cc2N)cc(OC)c1OC] 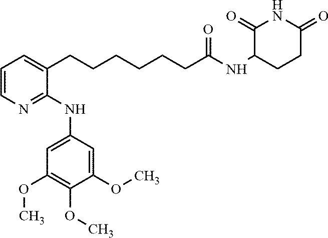 [SMILES: COc1cc(Nc2ncccc2CCCCCCC(=O)NC2CCC(=O)NC2=O)cc(OC)c1OC] 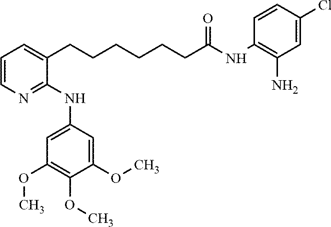 [SMILES: COc1cc(Nc2ncccc2CCCCCCC(=O)Nc2ccc(Cl)cc2N)cc(OC)c1OC]
.
